(19) United States
(12) Patent Application Publication
(10) Pub. No.: US 2024/0368637 A1
(43) Pub. Date: Nov. 7, 2024

(54) METHODS AND MICROORGANISMS FOR MAKING 1,4-BUTANEDIOL AND DERIVATIVES THEREOF FROM C1 CARBONS

(71) Applicant: PRECIGEN, INC., Germantown, MD (US)

(72) Inventors: Xinhua ZHAO, Germantown, MD (US); Tina HUYNH, Germantown, MD (US); Jeffrey ORTH, Germantown, MD (US); Lily Yuin CHAO, Germantown, MD (US); James KEALEY, Germantown, MD (US)

(73) Assignee: PRECIGEN, INC., Germantown, MD (US)

(21) Appl. No.: 18/661,283

(22) Filed: May 10, 2024

Related U.S. Application Data
(62) Division of application No. 17/447,568, filed on Sep. 13, 2021, now Pat. No. 12,018,310.
Application No. 17/447,568 is a division of application No. 16/492,411, filed on Sep. 9, 2019, now Pat. No. 11,155,837.
(60) Provisional application No. 62/470,953, filed on Mar. 14, 2017.

(51) Int. Cl. C12P 7/04; C12N 1/20; C12N 15/74

(52) U.S. Cl. CPC C12P 7/04; C12N 1/20; C12N 15/74

(57) ABSTRACT

Genetically modified microorganisms that have the ability to convert carbon substrates into chemical products such as 1,4-BDO are disclosed. For example, genetically modified methanotrophs that are capable of generating 1,4-BDO at high titers from a methane source are disclosed. Methods of making these genetically modified microorganisms and methods of using them are also disclosed.

### CROSS-REFERENCE TO RELATED APPLICATIONS

This application is a division of U.S. application Ser. No. 17/447,568, filed Sep. 13, 2021, which in turn is a division of U.S. application Ser. No. 16/492,411 (now U.S. Pat. No. 11,155,837), which is a U.S. national stage filing of International Application No. PCT/US2018/022204, filed Mar. 13, 2018, which in turn claims the priority benefit of U.S. Provisional Application No. 62/470,953, filed Mar. 14, 2017. The contents of each of the aforementioned is hereby incorporated by reference in their entireties.

### REFERENCE TO SEQUENCE LISTING

The present application contains a Sequence Listing which has been filed electronically in XML format and is hereby incorporated by reference in its entirety. Said XML copy, created on May 10, 2024, is named 409727.xml and is 113,226 bytes in size.

### BACKGROUND OF THE INVENTION

There has been great interest in generating fuels and chemicals by microbial fermentation in order to curb the use of fossil fuels. One such chemical of interest is 1,4-butanediol (1,4-BDO). 1,4-BDO is currently produced from petrochemical precursors, primarily acetylene, maleic anhydride, and propylene oxide.

1,4-BDO is also known as BD; 1,4-butylene glycol; 1,4-dihydroxybutane; BDO; butanediol; and 1,4-tetramethylene glycol and the IUPAC name is Butane-1,4-diol. Its chemical formula is C₄H₁₀O₂. The CAS number is 110-63-4.

1,4-BDO has a large number of industrial applications. For example, 1,4-BDO can be used as a monomer such as an acrylonitrile butadiene styrene (ABS) copolymer and polyurethane (PU), and may be converted into tetrahydrofuran, which may be used as a raw material for spandex fibers such as polytetramethylene ether glycol (PTMEG).

1,4-BDO is not known to be produced by any microorganism. 1,4-BDO is only produced with the aid of novel, engineered metabolic pathways or through chemical synthesis. See e.g., Adkins, J., “Engineering microbial chemical factories to produce renewable ‘biomonomers’”, Front. Microbiology (2012).

The present inventors disclose a way using genetically modified microorganisms, such as methanotrophs, in order to produce 1,4-BDO from C1 carbon feedstock.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY OF THE INVENTION

Disclosed herein are various genetically modified microorganisms that are capable of producing a desired organic compound, starting from a single carbon containing hydrocarbon molecule such as methane. Various methods of producing the desired organic compounds, including by using a genetically modified microorganism are disclosed.

For example, disclosed herein is a genetically modified microorganism capable of converting a C1 carbon source to a multicarbon product. The genetically modified microorganism can be genetically altered to produce different multicarbon products such as 1,4-butanediol. In some cases, the genetically modified microorganism can comprise one or more heterologous genes. The one or more heterologous genes can be one or more of pyruvate dehydrogenase (aceEF), citrate synthase (gltA), aconitate hydratase 1 (acnA), or isocitrate dehydrogenase (icdA). In some cases, the genetically modified microorganism will have all the genes above, however, at least one of the genes is endogenous to the microorganism. In some cases, all the genes can be heterologous to the microorganism. In some embodiments, the microorganism can further comprise one or more genes from a-ketoglutarate decarboxylase (kgd), 4-hyrobutyrate dehydrogenase (4hbD), 4-hydroxybutyryl-CoA transferase (Cat2), aldehyde dehydrogenase (Ald), or alcohol dehydrogenase (Adh). These additional genes can be one or more endogenous genes or one or more heterologous genes. In other embodiments, the microorganism can further comprise one or more genes from succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), or alcohol dehydrogenase (Adh). These additional genes can be one or more endogenous genes or one or more heterologous genes. In an additional embodiment, the microorganism can further comprise one or more genes from a-ketoglutarate decarboxylase (kgd), succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), or alcohol dehydrogenase (Adh). These additional genes can be one or more endogenous genes or one or more heterologous genes.

Also disclosed herein is a genetically modified microorganism that has one or more heterologous genes from a-ketoglutarate decarboxylase (kgd), 4-hyrobutyrate dehydrogenase (4hbD), 4-hydroxybutyryl-CoA transferase (Cat2), aldehyde dehydrogenase (Ald), or alcohol dehydrogenase (Adh). In some cases, the genetically modified microorganism will have all the genes, however, at least one of the genes is endogenous to the microorganism. In some cases, all the genes can be heterologous to the microorganism.

Also disclosed herein is a genetically modified microorganism that has one or more heterologous genes from succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), or alcohol dehydrogenase (Adh). In some cases, the genetically modified microorganism will have all the genes, however, at least one of the genes is endogenous to the microorganism. In some cases, all the genes can be heterologous to the microorganism.

Also disclosed herein is a genetically modified microorganism that has one or more heterologous genes from a-ketoglutarate decarboxylase (kgd), succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), or alcohol dehydrogenase (Adh). In some cases, the genetically modified microorganism will have all the genes, however, at least one of the genes is endogenous to the microorganism. In some cases, all the genes can be heterologous to the microorganism.

In some cases, the genetically modified microorganism disclosed throughout can further comprise one or more genes that are fumarate hydratase (fum) and/or fumarate reductase (frd).

The genes disclosed herein can be overexpressed, including both heterologous and endogenous genes.

The genetically modified microorganisms disclosed herein can be capable of converting a C1 carbon to a multicarbon product (e.g., 1,4-BDO) comprising one or more heterologous genes, where the heterologous gene encodes for an enzyme that can increase overall carbon flow from succinate to 1,4-BDO. Also, the genetically modified microorganisms disclosed herein are capable of converting a C1 carbon to a multicarbon product (e.g., 1,4-BDO) comprising one or more heterologous genes, where the heterologous gene encodes for an enzyme that can increase overall carbon flow from a-ketoglutarate to 1,4-BDO. Also disclosed herein is a genetically modified microorganism capable of converting a C1 carbon to a multi-carbon product comprising one or more heterologous genes, where the heterologous gene encodes for an enzyme that can increase overall carbon flow from oxaloacetate to succinate.

The genetically modified microorganism in some cases is a methylotroph, such as a methanotroph. The methanotroph can be from various genera such as Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, or Methylacidiphilum. A species that can be used is a Methylococcus capsulatus.

Also disclosed herein are vectors. The vectors can comprise any two or more genes of: pyruvate dehydrogenase (aceEF) gene, citrate synthase (gltA) gene, aconitate hydratase 1 (acnA) gene, isocitrate dehydrogenase (icdA) gene, a-ketoglutarate decarboxylase (kgd) gene, succinyl-CoA synthetase (sucC) gene, CoA-dependent succinate semialdehyde dehydrogenase (sucD) gene 4-hyrobutyrate dehydrogenase (4hbD) gene, 4-hydroxybutyryl-CoA transferase (cat2) gene, aldehyde dehydrogenase (ald), and/or alcohol dehydrogenase (adh) gene. For example, a vector as disclosed herein can comprise two or more genes of: pyruvate dehydrogenase (aceEF) gene, citrate synthase (gltA) gene, aconitate hydratase 1 (acnA) gene, and isocitrate dehydrogenase (icdA) gene. A vector as disclosed herein can also comprise two or more genes of: a-ketoglutarate decarboxylase (kgd) gene, 4-hyrobutyrate dehydrogenase (4hbD) gene, 4-hydroxybutyryl-CoA transferase (cat2) gene, aldehyde dehydrogenase (ald), and/or alcohol dehydrogenase (adh) gene. A vector as disclosed herein can also comprise two or more genes of: succinyl-CoA synthetase (sucC) gene, CoA-dependent succinate semialdehyde dehydrogenase (sucD) gene, 4-hyrobutyrate dehydrogenase (4hbD) gene, 4-hydroxybutyryl-CoA transferase (cat2) gene, aldehyde dehydrogenase (ald) gene, and/or alcohol dehydrogenase (adh) gene. In some cases, the vectors can comprise fumarate hydratase, and/or fumarate reductase.

Further disclosed herein are isolated polynucleic acids. For example, the isolated polynucleic acid can be a nucleotide sequence that is at least 85% identical to SEQ ID NO. 2. In some cases, the nucleotide sequence can encode for a protein that has 2-oxoglutarate decarboxylase activity. Also disclosed herein is an isolated polynucleic acid comprising a nucleotide sequence that is at least 85% identical to SEQ ID NO. 4 and/or a nucleotide sequence that is at least 87% identical to SEQ ID NO. 6. The nucleotide sequence can encode for a protein that has a-ketoglutarate decarboxylase activity. Disclosed is also an isolated polynucleic acid comprising a nucleotide sequence that is at least 86% identical to SEQ ID NO. 8. The nucleotide sequence can encode for a protein that has 2-oxoglutarate dehydrogenase El component activity. Further disclosed herein is an isolated polynucleic acid comprising a nucleotide sequence that is at least 79% identical to SEQ ID NO. 10. The nucleotide sequence can encode for a protein that has oxidoreductase activity. Disclosed also is an isolated polynucleic acid comprising a nucleotide sequence that is at least 60% identical to SEQ ID NO. 12 or 14. The nucleotide sequence can encode for a protein that has 4-hydroxybutyrate dehydrogenase activity. Also disclosed herein is an isolated polynucleic acid comprising a nucleotide sequence that is at least 60% identical to SEQ ID NO. 16; a nucleotide sequence that is at least 80% identical to SEQ ID NO. 18; and/or a nucleotide sequence that is at least 60% identical to SEQ ID NO. 20. The nucleotide sequence can encode for a protein that has 4-hydroxybutyrate CoA-transferase activity. Also disclosed herein is an isolated polynucleic acid comprising a nucleotide sequence that is at least 60% identical to SEQ ID NO. 22; a nucleotide sequence that is at least 84% identical to SEQ ID NO. 24; a nucleotide sequence that is SEQ ID NO. 26; a nucleotide sequence that is at least 60% identical to SEQ ID NO. 28; a nucleotide sequence that is at least 60% identical to SEQ ID NO. 30; a nucleotide sequence that is at least 60% identical to SEQ ID NO. 32; and/or a nucleotide sequence that is at least 82% identical to SEQ ID NO. 36. The nucleotide sequence can encode for a protein that has aldehyde dehydrogenase and/or alcohol dehydrogenase activity. Further disclosed herein is an isolated polynucleic acid comprising a nucleotide sequence that is at least 60% identical to SEQ ID NO. 34. The nucleotide sequence can encode for a protein that has ATP-dependent permease activity. Also disclosed herein is an isolated polynucleic acid comprising a nucleotide sequence that is at least 86% identical to SEQ ID NO. 38. The nucleotide sequence can encode for a protein that has succinyl-CoA synthetase, beta subunit activity. Further disclosed herein is an isolated polynucleic acid comprising a nucleotide sequence that is at least 86% identical to SEQ ID NO. 40; a nucleotide sequence that is at least 60% identical to SEQ ID NO. 42; and/or a nucleotide sequence that is at least 81% identical to SEQ ID NO. 44. The nucleotide sequence can encode for a protein that has succinyl-CoA synthetase, alpha subunit activity. Additionally disclosed herein is an isolated polynucleic acid comprising a nucleotide sequence that is at least 60% identical to SEQ ID NO. 48 and/or a nucleotide sequence that is at least 60% identical to SEQ ID NO. 50. The nucleotide sequence can encode for a protein that has fumarate hydratase activity. Also disclosed herein is an isolated polynucleic acid comprising a nucleotide sequence that is at least 83% identical to SEQ ID NO. 52; a nucleotide sequence that is at least 84% identical to SEQ ID NO. 54; a nucleotide sequence that is at least 60% identical to SEQ ID NO. 56; and/or a nucleotide sequence that is at least 81% identical to SEQ ID NO. 58. The nucleotide sequence can encode for a protein that has fumarate reductase activity.

Further disclosed herein is a method of making a genetically modified microorganism capable of converting a C1 carbon to a multicarbon product (e.g., 1,4-BDO) comprising contacting a microorganism with a nucleic acid that expresses or is capable of expressing at least one heterologous gene from i) aceEF, ii) lpdA, iii) gltA, iv) acnA, v) icdA, vi) kgd, vii) sucC, viii) sucD, ix) 4hbD, x) Cat2, xi) Ald, xii) Adh, xiii) Fum, xiv) Frd, or xv) any combination thereof. In some cases, the microorganism can be transformed with at least two, three, four, or five heterologous genes.

Additionally disclosed here is a method of making 1,4-BDO comprising (a) contacting a Cl carbon with a genetically modified microorganism capable of converting a C1 carbon into a multicarbon product (e.g., 1,4-BDO), where the microorganism comprises at least one heterologous gene encoding for: i) accEF, ii) lpdA, iii) gltA, iv) acnA, v) icdA, vi) kgd, vii) sucC, viii) sucD, ix) 4hbD, x) Cat2, xi) Ald, xii) Adh, xiii) Fum, xiv) Frd, or xv) any combination thereof; and (b) growing the microorganism to produce the multicarbon product. The multicarbon product can be 1,4-BDO. In some instances, the growing microorganism can be supplemented with exogenous GHB and/or exogenous a-ketoglutarate and/or exogenous succinate. After the multicarbon product (e.g., 1,4-BDO) is created, the multicarbon product can be recovered.

In some cases, the microorganisms described throughout can be fermented or grown for at least 96 hours.

The multicarbon product (e.g., 1,4-BDO) that is created can be further processed into other useful products such as tetrahydrofuran (THF). The 1,4-BDO that is made from the method disclosed throughout, can be converted into THE by contacting the 1,4-BDO from with a catalyst to produce THF. The 1,4-BDO can be isolated prior to contacting it with a catalyst to form THF. The catalyst used can be an acid catalyst, an alumina catalyst, a silica-alumina catalyst, an alumina-supported tungsten oxide catalyst, a heteropolyacid catalyst, or a zirconium sulfate catalyst. The THF produced by this method can be substantially pure and the method can also comprise recovering the THF. The recovered THF can also be further processed.

The multicarbon product (e.g., 1,4-BDO) that is created from the methods described throughout can also be further processed into chemicals such as polyurethanes. The 1,4-BDO that is made from the methods described throughout, can be converted into polyurethanes by condensing the 1,4-BDO with a dicarboxylic acid/anhydride. The 1,4-BDO can be isolated prior to condensing it with a dicarboxylic acid/anhydride. In some cases, dicarboxylic acid/anhydride can be aliphatic or aromatic. The polyurethanes produced can be substantially pure. The polyurethanes produced by this method can be recovered and optionally further processed.

The multicarbon product (e.g., 1,4-BDO) that is created from the methods described throughout can also be further processed into chemicals such as polybutylene terephthalate (PBT). The 1,4-BDO that is made from the methods described throughout, can be converted into PBT by transesterfying the 1,4-BDO. The 1,4-BDO can be isolated prior to transesterification. In some cases, the transesterification can be done by contacting the 1,4-BDO with dimethyl terephthalate (DMT). The PBT produced can be substantially pure. The PBT produced by this method can be recovered and optionally further processed.

Also disclosed herein is a genetically modified microorganism capable of converting a C1 carbon source to multicarbon product (e.g., 1,4-BDO) comprising one or more heterologous genes, where the one or more heterologous genes is a gene from one or more of the following groups: GROUP 1: pyruvate dehydrogenase (accEF), citrate synthase (gltA), aconitate hydratase 1 (acnA), isocitrate dehydrogenase (icdA); fumarate hydratase (fum); and/or fumarate reductase (frd); GROUP 2: α-ketoglutarate decarboxylase (kgd), 4-hyrobutyrate dehydrogenase (4hbD), 4-hydroxybutyryl-CoA transferase (Cat2), aldehyde dehydrogenase (Ald), and/or alcohol dehydrogenase (Adh); or GROUP 3: succinyl-CoA synthetase (SucC), CoA-dependent succinate semialdehyde dehydrogenase (SucD), 4-hyrobutyrate dehydrogenase (4hbD), 4-hydroxybutyryl-CoA transferase (Cat2), aldehyde dehydrogenase (Ald), and/or alcohol dehydrogenase (Adh). The heterologous gene can be overexpressed. In some cases, one or more of the genes or groups of genes can be endogenous to the microorganism. For example, in one case, one or more of the genes in GROUP 1 can be heterologous, and the microorganism contains endogenous genes belonging to GROUP 2, GROUP 3, or both GROUP 2 and 3. In another instance, one or more genes from GROUP 2 can be heterologous, and the microorganism contains endogenous genes belonging to GROUP 1, GROUP 3, or both GROUP 1 and 3. In another example, one or more genes from GROUP 3 can be heterologous, and the microorganism contains endogenous genes belonging to GROUP 1, GROUP 2, or both GROUP 1 and 2. In another example, one or more genes from GROUP 1 and 2, can be heterologous whereas one or more genes from GROUP 3 can be endogenous. In other case, one or more genes from GROUP 1 and 3 can be heterologous, wherein one or more genes from GROUP 2 can be endogenous. In other cases, one or more genes from GROUP 1, 2, and 3 can be heterologous. Even if there is one or more heterologous genes from any of the GROUPs, this does not mean the microorganism does not contain endogenous genes from that same group. For example, a microorganism can have both a heterologous and endogenous gene from GROUP 1.

Further disclosed herein is a method of making succinate comprising (a) contacting a C₁ carbon with a genetically modified microorganism, wherein the genetically modified microorganism comprises a heterologous fumarate hydratase, fumarate reductase, or any combination thereof; and (b) growing the genetically modified microorganism to produce succinate. In some cases, the genetically modified microorganism can comprise a pyruvate dehydrogenase (accEF), citrate synthase (gltA), aconitate hydratase 1 (acnA), and/or isocitrate dehydrogenase (icdA). In some cases, the succinate from (b) is isolated. In some cases, the succinate is converted into 1,4-BDO.

In some cases, the genetically modified microorganism is grown for at least 96 hours.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a metabolic pathway from methane (CH4) to 1,4-BDO. The circled part shows the pathway from pyruvate to α-ketoglutarate, which is common to both the α-ketoglutarate to 1,4-BDO pathway and the succinate to 1,4-BDO pathway. The group of enzymes that is responsible for converting pyruvate into α-ketoglutarate is referred to as GROUP 1 throughout. GROUP 1 comprises at least the following enzymes: pyruvate dehydrogenase (aceEF), citrate synthase (gltA), aconitate hydratase 1 (acnA), and isocitrate dehydrogenase (icdA).

FIG. 2 shows the α-ketoglutarate to 1,4-BDO pathway. α-ketoglutarate is converted to succinate semialdehyde through an α-ketoglutarate decarboxylase (kgd). Succinate semialdehyde is then converted to gamma hydroxybutyrate (GHB) by 4-hydroxybutyrate dehydrogenase (4hbD). GHB is then converted to 4-hydroxybutyryl CoA by 4-hydroxybutyrate CoA transferase (cat2). 4-hydroxybutyryl CoA is then converted to 4-hydroxybutyraldehyde by aldehyde dehydrogenase (Ald) through the use of NAD (P) H. 4-hydroxybutyraldehyde is converted to 1,4-BDO by alcohol dehydrogenase through the use of NAD (P) H. The group of enzymes that is responsible for converting α-ketoglutarate to 1,4-BDO is referred to as GROUP 2 throughout.

FIG. 3 shows the succinate to 1,4-BDO pathway. Succinate is converted to succinyl CoA by Succinyl CoA synthease beta subunit (sucC). Succinyl CoA is then converted to succinate semialdehyde by succinyl CoA synthease alpha subunit (sucD). Succinate semialdehyde is then converted to γ-hydroxybutyrate (ghb) through the use of 4-hydroxybutyrate dehydrogenase (4hbD). GHB is then converted to 4-hydroxybutyryl CoA by 4-hydroxybutyrate CoA transferase (cat2). 4-hydroxybutyryl CoA is then converted to 4-hydroxybutyraldehyde by aldehyde dehydrogenase (ald) through the use of NAD (P) H. 4-hydroxybutyraldehyde is converted to 1,4-BDO by alcohol dehydrogenase through the use of NAD (P) H. The group of enzymes that is responsible for converting succinate to 1,4-BDO is referred to as GROUP 3 throughout.

FIG. 4 shows the production of 1,4-BDO by strain XZ76. StainXZ76 produces over 1000 ug/L of 1,4-BDO when supplemented with 400 mg/L of α-ketoglutarate and 50 mg/L GHB. Supplementing the media with only 400 mg/L of α-ketoglutarate produced approximately 100 ug/L of 1,4-BDO, whereas no supplementation did not produce any 1,4-BDO.

FIG. 5 shows the production of α-ketoglutarate from various strains overexpression pyruvate dehydrogenase (accEF), citrate synthase (gltA), aconitate hydratase 1 (acnA), or isocitrate dehydrogenase (icdA). Overexpression of icdA lead to a large increase α-ketoglutarate production.

FIG. 6 shows 1,4-BDO production from two top strains XZ79 and XZ344 with or without the presence α-ketoglutarate or succinic acid (collectively “feed”). XZ79 and XZ344 were able both able to produce detectable 1,4 BDO directly from methane in a shake bottle experiment. XZ79 and XZ344 both produced detectable amount of 1,4 BDO directly from methane whereas supplementing the media with feed increased the 1,4 BDO productivity.

FIG. 7 shows a metabolic pathway from methane (CH4) to 1,4-BDO. The circled part shows the pathway from oxaloacetate and malyl-CoA to succinate, the conversions which are facilitated in part by fumarate hydratase (fum) and fumarate reductase (frd).

FIG. 8 shows methanotrophs expressing Frd genes under the control of different promoters. The MBC1960 strain expressed a heterologous E. coli FrdA, FrdB, FrdC, and FrdD under the control of a pmxaF promoter. The MBC1970 strain expresses the same genes except they are under the control of a J23111 promoter whereas MBC1985 expresses the genes under the control of a J23100 promoter. The MBC1970 and MBC1985 strains produced significantly more succinate than its parent control strain.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

1,4-BDO is a high value chemical that is currently produced mainly from petroleum sources. There has been some interest in recent years, however, to produce 1,4-BDO by fermentation. Fermentation typically involves taking a carbon source (usually sugar) and fermenting it using a microorganism that is capable of converting the carbon source into a desired product.

Costs to produce chemicals such as 1,4-BDO by fermentation typically depends on the cost of the carbon source used. Sugars are generally higher cost carbon sources that also result in a decrease of food supply. One carbon source is currently extremely cost-effective and abundant is natural gas. The primary source of carbon within natural gas is methane, a C1 carbon. By using cheap carbon sources such as methane, 1,4-BDO can be produced economically. However, the challenge lies engineering fermentation methods and microorganisms to efficiently convert cheap carbon sources, such as methane, into 1,4-BDO using a fermentation process.

As discussed above, 1,4-BDO is not known to be naturally produced by any microorganisms. Thus, intense and extensive genetic engineering is required to produce 1,4-BDO.

Described herein are genetically modified microorganisms, e.g., methylotrophs, for example, methanotrophs, that can convert a C1 carbon substrate, such as methane, into desired products. Some of the genetically modified microorganisms disclosed herein have been designed and altered to efficiently produce 1,4-BDO, multiple folds over what is expected to be produced. For example, methanotrophs, which do not normally produce 1,4-BDO, can be genetically engineered to efficiently produce large quantities of 1,4-BDO. Additionally some of the genetically modified microorganisms disclosed herein can be used to convert a C1 carbon substrate into 1,4-BDO and subsequently into polyurethanes, tetrahydrofurans (THF), polybutylene terephthalates (PBT), or other desired products. These genetically modified microorganisms and the novel methods of fermentation and uses thereof are described herein.

### Definitions

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

The term “genetic modification” or “genetically modified” and their grammatical equivalents as used herein can refer to one or more alterations of a nucleic acid, e.g., the nucleic acid within a microorganism's genome. For example, genetic modification can refer to alterations, additions, and/or deletion of nucleic acid (e.g., whole genes or fragments of genes).

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression (e.g., mRNA and/or protein expression) of the gene. Disrupting can also include inhibitory technology, such as shRNA, siRNA, microRNA, dominant negative, or any other means to inhibit functionality or expression of a gene or protein.

The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease).

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “substantially pure” and its grammatical equivalents as used herein can mean that a particular substance does not contain a majority of another substance. For example, “substantially pure 1,4-BDO” can mean at least 90% 1,4-BDO. In some instances, “substantially pure 1,4-BDO” can mean at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, or 99.9999% 1,4-BDO. For example, substantially pure 1,4-BDO can mean at least 70% 1,4-BDO. In some cases, substantially pure 1,4-BDO can mean at least 75% 1,4-BDO. In some cases, substantially pure 1,4-BDO can mean at least 80% 1,4-BDO. In some cases, substantially pure 1,4-BDO can mean at least 85% 1,4-BDO. In some cases, substantially pure 1,4-BDO can mean at least 90% 1,4-BDO. In some cases, substantially pure 1,4-BDO can mean at least 91% 1,4-BDO. In some cases, substantially pure 1,4-BDO can mean at least 92% 1,4-BDO. In some cases, substantially pure 1,4-BDO can mean at least 93% 1,4-BDO. In some cases, substantially pure 1,4-BDO can mean at least 94% 1,4-BDO. In some cases, substantially pure 1,4-BDO can mean at least 95% 1,4-BDO. In some cases, substantially pure 1,4-BDO can mean at least 96% 1,4-BDO. In some cases, substantially pure 1,4-BDO can mean at least 97% 1,4-BDO. In some cases, substantially pure 1,4-BDO can mean at least 98% 1,4-BDO. In some cases, substantially pure 1,4-BDO can mean at least 99% 1,4-BDO.

The term “heterologous” and its grammatical equivalents as used herein can mean “derived from a different species.” For example, a “heterologous gene” can mean a gene that is from a different species. In some instances, as “a methanotroph comprising a heterologous gene” can mean that the methanotroph contains a gene that is not from the same methanotroph. The gene can be from a different microorganism such as yeast or from a different species such as a different methanotroph species.

The term “substantially similar” and its grammatical equivalents in reference to another sequence as used herein can mean at least 50% identical. In some instances, the term substantially similar refers to a sequence that is 55% identical. In some instances, the term substantially similar refers to a sequence that is 60% identical. In some instances, the term substantially similar refers to a sequence that is 65% identical. In some instances, the term substantially similar refers to a sequence that is 70% identical. In some instances, the term substantially similar refers to a sequence that is 75% identical. In some instances, the term substantially similar refers to a sequence that is 80% identical. In other instances, the term substantially similar refers to a sequence that is 81% identical. In other instances, the term substantially similar refers to a sequence that is 82% identical. In other instances, the term substantially similar refers to a sequence that is 83% identical. In other instances, the term substantially similar refers to a sequence that is 84% identical. In other instances, the term substantially similar refers to a sequence that is 85% identical. In other instances, the term substantially similar refers to a sequence that is 86% identical. In other instances, the term substantially similar refers to a sequence that is 87% identical. In other instances, the term substantially similar refers to a sequence that is 88% identical. In other instances, the term substantially similar refers to a sequence that is 89% identical. In some instances, the term substantially similar refers to a sequence that is 90% identical. In some instances, the term substantially similar refers to a sequence that is 91% identical. In some instances, the term substantially similar refers to a sequence that is 92% identical. In some instances, the term substantially similar refers to a sequence that is 93% identical. In some instances, the term substantially similar refers to a sequence that is 94% identical. In some instances, the term substantially similar refers to a sequence that is 95% identical. In some instances, the term substantially similar refers to a sequence that is 96% identical. In some instances, the term substantially similar refers to a sequence that is 97% identical. In some instances, the term substantially similar refers to a sequence that is 98% identical. In some instances, the term substantially similar refers to a sequence that is 99% identical. In some instances, the term substantially similar refers to a sequence that is 100% identical. In order to determine the percentage of identity between two sequences, the two sequences are aligned, using for example the alignment method of Needleman and Wunsch (J. Mol. Biol., 1970, 48:443), as revised by Smith and Waterman (Adv. Appl. Math., 1981, 2:482) so that the highest order match is obtained between the two sequences and the number of identical amino acids/nucleotides is determined between the two sequences. For example, methods to calculate the percentage identity between two amino acid sequences are generally art recognized and include, for example, those described by Carillo and Lipton (SIAM J. Applied Math., 1988, 48:1073) and those described in Computational Molecular Biology, Lesk, c.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12:387) BLASTP, BLASTN and FASTA (Altschul et al., J. Molec. Biol., 1990:215:403). A particularly preferred method for determining the percentage identity between two polypeptides involves the Clustal W algorithm (Thompson, J D, Higgines, D G and Gibson T J, 1994, Nucleic Acid Res 22 (22): 4673-4680 together with the BLOSUM 62 scoring matrix (Henikoff S & Henikoff, J G, 1992, Proc. Natl. Acad. Sci. USA 89:10915-10919 using a gap opening penalty of 10 and a gap extension penalty of 0.1, so that the highest order match obtained between two sequences wherein at least 50% of the total length of one of the two sequences is involved in the alignment.

The term “C1 carbon” or “C1 carbon substrates” and their grammatical equivalents as used herein can be used interchangeably and can mean any organic compound that contains a single carbon atom. Examples can include, but are not limited to, CO, CH₄, and/or CO₂.

### I. Genetically Modified Microorganisms and Methods of Making the Same

1,4-BDO is not known to be naturally produced by any organism. However, this application discloses genetically modified microorganisms that can produce 1,4-BDO, and can do so at high levels. For example, disclosed herein are microorganisms that do not naturally produce 1,4-BDO but can be genetically modified to synthesize 1,4-BDO, including at commercially feasible levels.

### Microorganisms

The microorganisms described herein can produce multicarbon products from a C1 carbon substrate, such as, but not limited to CO, CO₂, and CH₄. This however does not mean that these microorganisms use solely C1 carbons. Some of the microorganisms disclosed herein can be made to utilize additional carbon substrates, including carbon substrates that the microorganism naturally uses in addition to other carbon substrates.

The microorganisms disclosed herein can be a prokaryote or eukaryote. Additionally, other microorganisms such as bacteria, yeast, or algae can be used.

Some microorganisms can use a C1 carbon to generate a desired product. For example, some of the microorganisms that can convert C1 carbon substrates into desired products can be a microorganism that is capable of using natural gas as a carbon substrate. In some cases, the microorganism can use the methane contained within the natural gas a as a carbon source to make desired products. One type of microorganism that uses C1 carbon substrates to form desired organic compounds are methylotrophs, such as methanotrophs. The methanotrophs that can be particularly useful include methanotrophs from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, Methylacidiphilum, or any combinations thereof. Methanotrophs from the genus Methylococcus can be particularly useful. When a methanotroph from the genus Methylococcus is used, a methanotroph from the species Methylococcus capsulatus can be used.

Some microorganisms are microorganisms that are capable of using CO₂ as a carbon substrate. For instance, the microorganisms can be a methanogen. Microorganisms that are capable of using CO₂ as a substrate can contain chlorophyll. One type of microorganism that uses CO₂ to form desired organic compounds are algae. Another type of microorganism that can use CO₂ as a substrate is a cyanobacterium.

Some microorganisms that can convert C1 carbon substrates into desired products can be a microorganism that is capable of using CO as a carbon substrate. Anaerobic microorganism can typically process CO and therefore can be used herein. One type of microorganism that uses CO to form desired organic compounds are bacterium such as Clostridium. These microorganism can be genetically modified into making substantial amounts of 1,4-BDO.

### Enzymes

In order to genetically engineer certain microorganisms to produce certain useful products such as 1,4-BDO, microorganisms can be transformed with one or more genes that encode for specific enzymes. These genes can be heterologous to the microorganism.

It is also contemplated that any and all genes disclosed herein can be overexpressed. This can lead to an overexpression of the gene but also lead to an increase of the actual polypeptide levels. For example, when a microorganism or vector comprises a gene (e.g., a heterologous gene), the gene can be overexpressed. This is done typically by using a promoter that is highly expressed or inserting multiple copies of the gene.

For example, the genetically modified microorganism can comprise one or more nucleic acids encoding for an enzyme capable of catalyzing one or more of the reactions: i) methane to methanol; ii) methanol to formaldehyde; iii) formaldehyde to pyruvate; iv) pyruvate to acetyl CoA; v) acetyl CoA to citrate; vi) citrate to isocitrate; vii) isocitrate to α-ketoglutarate; viii) α-ketoglutarate to succinyl CoA; ix) succinate CoA to succinate; x) oxaloacetate and/or malyl-CoA to L-malate; xi) L-malate to fumarate; and/or xii) fumarate to succinate. For example, the genetically modified microorganism can comprise one or more genes including but not limited to: pyruvate dehydrogenase (accEF), citrate synthase (gltA), aconitate hydratase 1 (acnA), and isocitrate dehydrogenase (icdA), α-ketoglutaratc dehydrogenase, succinyl-CoA synthetase; fumarate hydratase (fum); fumarate reductase (frd); or any combination thereof. Depending on the substrate used, any of the products of the pathway can be made by increasing or decreasing the expression of the enzymes that promote the formation of the desired product. Some of the nucleic acids can be endogenous to the microorganism and some of the nucleic acids can be heterologous to the microorganism.

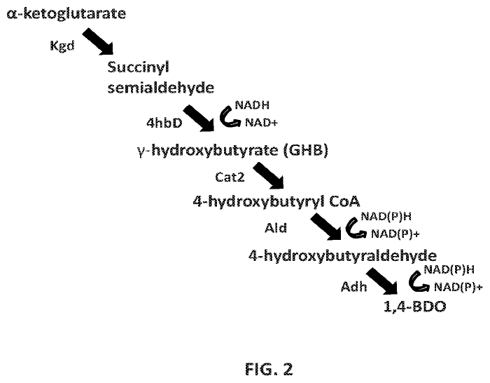

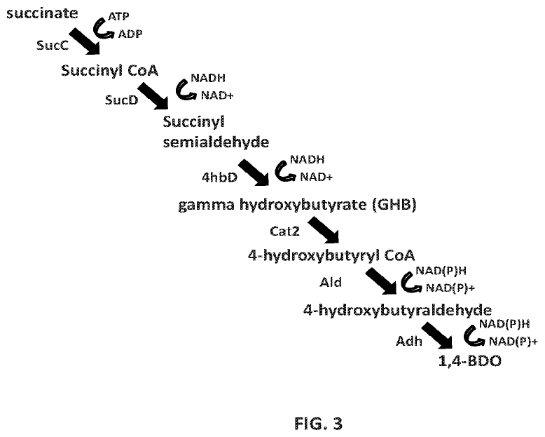

Further, in order to create a microorganism that can produce a multicarbon product such as 1,4-BDO, one or more genes (e.g., heterologous genes) can be transformed/transfected (i.e., inserted) into the microorganism (transiently or stably). At least one genes of any of the i) GROUP 2: α-ketoglutarate to 1,4-BDO (see e.g., FIG. 2) and ii) GROUP 3: succinate to 1,4-BDO pathway (see e.g., FIG. 3) can be used to generate a multicarbon substrate such as 1,4-BDO at efficiently levels. A microorganism containing one or more genes from one or both pathway can be placed inside a microorganism in order to produce a multicarbon substrate such as 1,4-BDO. For example, a gene from GROUP 2 (the α-ketoglutarate to 1,4-BDO pathway) can be used in order to produce multicarbon substrate such as 1,4-BDO from a microorganism. Alternatively, a gene from GROUP 3 (the succinate to 1,4-BDO pathway) can be used in order to produce a multicarbon substrate such as 1,4-BDO from a microorganism. Another way to produce multicarbon substrate such as 1,4-BDO from a microorganism can be expressing one or more genes from both GROUP 2 (the α-ketoglutarate to 1,4-BDO pathway) and GROUP 3 (the succinate to 1,4-BDO pathway). In some case, all of the genes from GROUP 2 can be used. In other cases, all of the genes from GROUP 3 can be used. In other instances, all the genes from both GROUPS 2 and 3 can be used. In some cases, the microorganism can comprise a fumarate hydratase (fum) and/or fumarate reductase (frd). In some cases, some of the genes from the groups can be an endogenous gene.

In some cases, when the microorganism utilizes the α-ketoglutarate to 1,4-BDO pathway, the microorganism can comprise α-ketoglutarate decarboxylase (kgd), which is an enzyme that is capable of converting α-ketoglutarate to succinate semialdehyde. In some cases, the microorganism can comprise a 4-hydroxybutyrate dehydrogenase (4hbD), which is an enzyme that converts succinate semialdehyde to γ-hydroxybutyrate (GHB). In some cases, the microorganism can comprise a 4-hydroxybutyrate CoA transferase (cat2), which is an enzyme that converts γ-hydroxybutyrate (GHB) to 4-hydroxybutyryl CoA. In some cases, the microorganism can comprise an aldehyde dehydrogenase (ald), which is an enzyme that converts 4-hydroxybutyryl CoA to 4-hydroxybutyraldehyde. In some cases, the microorganism can comprise an alcohol dehydrogenase (adh), which is an enzyme that converts 4-hydroxybutyraldehyde to 1,4-BDO. In some cases, all the enzymes from the α-ketoglutarate to 1,4-BDO pathway can be used. One or more of these enzymes can be endogenous to the microorganism.

Described here are microorganisms used to make a multicarbon product such as 1,4-BDO from a C1 carbon (e.g., methane). In some cases, the microorganism herein can be transformed with a gene encoding for one or more of the following: i) α-ketoglutarate decarboxylase (kgd); ii) 4-hydroxybutyrate dehydrogenase (4hbD); iii) 4-hydroxybutyrate CoA transferase (cat2); (iv) aldehyde dehydrogenase (ald); (v) alcohol dehydrogenase (adh); or (vi) any combination thereof. These genes can be heterologous to the microorganism. The genes can also encode for an enzyme that can conduct the chemical conversions as described above. For example, the aldehyde dehydrogenase (Ald) can be an enzyme that converts 4-hydroxybutyryl CoA to 4-hydroxybutyraldehyde, whereas the alcohol dehydrogenase (adh) can be an enzyme that converts 4-hydroxybutyraldehyde to 1,4-BDO.

In some cases, the microorganism can comprise a fumarate hydratase (fum), which is an enzyme that converts L-malate to fumarate. In some cases, the microorganism can comprise a fumarate reductase (frd), which is an enzyme that converts fumarate to succinate.

When an α-ketoglutarate decarboxylase (kgd) is used the α-ketoglutarate decarboxylase can be from a bacteria (e.g., a gram positive bacterium or a bacterium that is neither gram positive or gram negative), such as from the genus Mycobacterium, Corynebacterium and/or Rhodococcus. For example, an α-ketoglutarate decarboxylase (kgd) can be from the species Mycobacterium bovis, Mycobacterium tuberculosis, Corynebacterium terpenotabidum, and/or Rhbodococcus jostii.

The α-ketoglutarate decarboxylase (kgd) can comprise an amino acid sequence that is substantially similar to any one of SEQ ID NOs. 1, 3, 5, or 7. For example, the α-ketoglutarate decarboxylase can comprise an amino acid sequence that is at least 60% identical to any one of SEQ ID NOs. 1, 3, 5, or 7. For example, the α-ketoglutarate decarboxylase can comprise an amino acid sequence that is at least 65% identical to any one of SEQ ID NOs. 1, 3, 5, or 7. In some cases, the α-ketoglutarate decarboxylase can comprise an amino acid sequence that is at least 70% identical to any one of SEQ ID NOs. 1, 3, 5, or 7. For example, the α-ketoglutarate decarboxylase can comprise an amino acid sequence that is at least 75% identical to any one of SEQ ID NOs. 1, 3, 5, or 7. In some cases, the α-ketoglutarate decarboxylase can comprise an amino acid sequence that is at least 80% identical to any one of SEQ ID NOs. 1, 3, 5, or 7. For example, the α-ketoglutarate decarboxylase can comprise an amino acid sequence that is at least 85% identical to any one of SEQ ID NOs. 1, 3, 5, or 7. In some cases, the α-ketoglutarate decarboxylase can comprise an amino acid sequence that is at least 90% identical to any one of SEQ ID NOs. 1, 3, 5, or 7. In some cases, the α-ketoglutarate decarboxylase can comprise an amino acid sequence that is at least 91% identical to any one of SEQ ID NOs. 1, 3, 5, or 7. In some cases, the α-ketoglutarate decarboxylase can comprise an amino acid sequence that is at least 92% identical to any one of SEQ ID NOs. 1, 3, 5, or 7. In some cases, the α-ketoglutarate decarboxylase can comprise an amino acid sequence that is at least 93% identical to any one of SEQ ID NOs. 1, 3, 5, or 7. In some cases, the α-ketoglutarate decarboxylase can comprise an amino acid sequence that is at least 94% identical to any one of SEQ ID NOs. 1, 3, 5, or 7. In some cases, the α-ketoglutarate decarboxylase can comprise an amino acid sequence that is at least 95% identical to any one of SEQ ID NOs. 1, 3, 5, or 7. In some cases, the α-ketoglutarate decarboxylase can comprise an amino acid sequence that is at least 96% identical to any one of SEQ ID NOs. 1, 3, 5, or 7. In some cases, the α-ketoglutarate decarboxylase can comprise an amino acid sequence that is at least 97% identical to any one of SEQ ID NOs. 1, 3, 5, or 7. In some cases, the α-ketoglutarate decarboxylase can comprise an amino acid sequence that is at least 98% identical to any one of SEQ ID NOs. 1, 3, 5, or 7. In some cases, the α-ketoglutarate decarboxylase can comprise an amino acid sequence that is at least 99% identical to any one of SEQ ID NOs. 1, 3, 5, or 7. In some cases, the α-ketoglutarate decarboxylase can comprise an amino acid sequence that is any one of SEQ ID NOs. 1, 3, 5, or 7.

When a 4-hydroxybutyrate dehydrogenase (4hbD) is desired the 4-hydroxybutyrate dehydrogenase can be from a bacterium (e.g., a gram negative or positive bacterium), such as from the genus Escherichia, Porphyromonas, and/or Clostridium. For example, a 4-hydroxybutyrate dehydrogenase can be from the species Escherichia coli, Porphyromonas gingivalis, and/or Clostridium kluyveri.

The 4-hydroxybutyrate dehydrogenase can comprise an amino acid sequence that is substantially similar to SEQ ID NO. 11 or 13. For example, the 4-hydroxybutyrate dehydrogenase can comprise an amino acid sequence that is at least 60% identical to SEQ ID NO. 11 or 13. For example, the 4-hydroxybutyrate dehydrogenase can comprise an amino acid sequence that is at least 65% identical to SEQ ID NO. 11 or 13. In some cases, the 4-hydroxybutyrate dehydrogenase can comprise an amino acid sequence that is at least 70% identical to SEQ ID NO. 11 or 13. For example, the 4-hydroxybutyrate dehydrogenase can comprise an amino acid sequence that is at least 75% identical to SEQ ID NO. 11 or 13. In some cases, the 4-hydroxybutyrate dehydrogenase can comprise an amino acid sequence that is at least 80% identical to SEQ ID NO. 11 or 13. For example, the 4-hydroxybutyrate dehydrogenase can comprise an amino acid sequence that is at least 85% identical to SEQ ID NO. 11 or 13. In some cases, the 4-hydroxybutyrate dehydrogenase can comprise an amino acid sequence that is at least 90% identical to SEQ ID NO. 11 or 13. In some cases, the 4-hydroxybutyrate dehydrogenase can comprise an amino acid sequence that is at least 91% identical to SEQ ID NO. 11 or 13. In some cases, the 4-hydroxybutyrate dehydrogenase can comprise an amino acid sequence that is at least 92% identical to SEQ ID NO. 11 or 13. In some cases, the 4-hydroxybutyrate dehydrogenase can comprise an amino acid sequence that is at least 93% identical to SEQ ID NO. 11 or 13. In some cases, the 4-hydroxybutyrate dehydrogenase can comprise an amino acid sequence that is at least 94% identical to SEQ ID NO. 11 or 13. In some cases, the 4-hydroxybutyrate dehydrogenase can comprise an amino acid sequence that is at least 95% identical to SEQ ID NO. 11 or 13. In some cases, the 4-hydroxybutyrate dehydrogenase can comprise an amino acid sequence that is at least 96% identical to SEQ ID NO. 11 or 13. In some cases, the 4-hydroxybutyrate dehydrogenase can comprise an amino acid sequence that is at least 97% identical to SEQ ID NO. 11 or 13. In some cases, the 4-hydroxybutyrate dehydrogenase can comprise an amino acid sequence that is at least 98% identical to SEQ ID NO. 11 or 13. In some cases, the 4-hydroxybutyrate dehydrogenase can comprise an amino acid sequence that is at least 99% identical to SEQ ID NO. 11 or 13. In some cases, the 4-hydroxybutyrate dehydrogenase can comprise an amino acid sequence that is SEQ ID NO. 11 or 13.

When a 4-hydroxybutyrate CoA transferase (Cat2) is desired the 4-hydroxybutyrate CoA transferase can be from a bacterium (e.g., a gram negative or positive bacterium), such as from the genus Porphyromonas, and/or Clostridium. For example, a 4-hydroxybutyrate CoA transferase can be from the species Porphyromonas gingivalis and/or Clostridium acetobutylicum.

The 4-hydroxybutyrate CoA transferase can comprise an amino acid sequence that is substantially similar to any one of SEQ ID NOs. 15, 17, or 19. For example, the 4-hydroxybutyrate CoA transferase can comprise an amino acid sequence that is at least 60% identical to any one of SEQ ID NOs. 15, 17, or 19. For example, the 4-hydroxybutyrate CoA transferase can comprise an amino acid sequence that is at least 65% identical to any one of SEQ ID NOs. 15, 17, or 19. In some cases, the 4-hydroxybutyrate CoA transferase can comprise an amino acid sequence that is at least 70% identical to any one of SEQ ID NOs. 15, 17, or 19. For example, the 4-hydroxybutyrate CoA transferase can comprise an amino acid sequence that is at least 75% identical to any one of SEQ ID NOs. 15, 17, or 19. In some cases, the 4-hydroxybutyrate CoA transferase can comprise an amino acid sequence that is at least 80% identical to any one of SEQ ID NOs. 15, 17, or 19. For example, the 4-hydroxybutyrate CoA transferase can comprise an amino acid sequence that is at least 85% identical to any one of SEQ ID NOs. 15, 17, or 19. In some cases, the 4-hydroxybutyrate CoA transferase can comprise an amino acid sequence that is at least 90% identical to any one of SEQ ID NOs. 15, 17, or 19. In some cases, the 4-hydroxybutyrate CoA transferase can comprise an amino acid sequence that is at least 91% identical to any one of SEQ ID NOs. 15, 17, or 19. In some cases, the 4-hydroxybutyrate CoA transferase can comprise an amino acid sequence that is at least 92% identical to any one of SEQ ID NOs. 15, 17, or 19. In some cases, the 4-hydroxybutyrate CoA transferase can comprise an amino acid sequence that is at least 93% identical to any one of SEQ ID NOs. 15, 17, or 19. In some cases, the 4-hydroxybutyrate CoA transferase can comprise an amino acid sequence that is at least 94% identical to any one of SEQ ID NOs. 15, 17, or 19. In some cases, the 4-hydroxybutyrate CoA transferase can comprise an amino acid sequence that is at least 95% identical to any one of SEQ ID NOs. 15, 17, or 19. In some cases, the 4-hydroxybutyrate CoA transferase can comprise an amino acid sequence that is at least 96% identical to any one of SEQ ID NOs. 15, 17, or 19. In some cases, the 4-hydroxybutyrate CoA transferase can comprise an amino acid sequence that is at least 97% identical to any one of SEQ ID NOs. 15, 17, or 19. In some cases, the 4-hydroxybutyrate CoA transferase can comprise an amino acid sequence that is at least 98% identical to any one of SEQ ID NOs. 15, 17, or 19. In some cases, the 4-hydroxybutyrate CoA transferase can comprise an amino acid sequence that is at least 99% identical to any one of SEQ ID NOs. 15, 17, or 19. In some cases, the 4-hydroxybutyrate CoA transferase can comprise an amino acid sequence that is any one of SEQ ID NOs. 15, 17, or 19.

When an aldehyde dehydrogenase (Ald) and/or an alcohol dehydrogenase (Adh) is desired the aldehyde dehydrogenase and/or alcohol dehydrogenase can be from a bacterium (e.g., a gram negative or positive bacterium), such as from the genus Escherichia, Acinetobacter, Porphyromonas, and/or Clostridium, or from a yeast such as from the genus Saccharomyces. For example, a 4-hydroxybutyrate CoA transferase can be from the species Escherichia coli, Acinetobacter baylyi, Porphyromonas gingivalis, Clostridium acetobutylicum and/or Saccharomyces cerevisiae. In some cases, more than one aldehyde dehydrogenase and/or alcohol dehydrogenase can be used.

The aldehyde dehydrogenase and/or alcohol dehydrogenase can comprise an amino acid sequence that is substantially similar to any one of SEQ ID NOs. 21, 23, 25, 27, 29, 31, 33, or 35. For example, the aldehyde dehydrogenase and/or alcohol dehydrogenase can comprise an amino acid sequence that is at least 60% identical to any one of SEQ ID NOs. 21, 23, 25, 27, 29, 31, 33, or 35. For example, the aldehyde dehydrogenase and/or alcohol dehydrogenase can comprise an amino acid sequence that is at least 65% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 70% identical to any one of SEQ ID NOs. 21, 23, 25, 27, 29, 31, 33, or 35. For example, the aldehyde dehydrogenase and/or alcohol dehydrogenase can comprise an amino acid sequence that is at least 75% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 80% identical to any one of SEQ ID NOs. 21, 23, 25, 27, 29, 31, 33, or 35. For example, the aldehyde dehydrogenase and/or alcohol dehydrogenase can comprise an amino acid sequence that is at least 85% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 90% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 91% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 92% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 93% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 94% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 95% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 96% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 97% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 98% identical to any one of SEQ ID NOs. 21, 23, 25, 27, 29, 31, 33, or 35. In some cases, the aldehyde dehydrogenase and/or alcohol dehydrogenase can comprise an amino acid sequence that is at least 99% identical to any one of SEQ ID NOs. 21, 23, 25, 27, 29, 31, 33, or 35. In some cases, the aldehyde dehydrogenase and/or alcohol dehydrogenase can comprise an amino acid sequence that is any one of SEQ ID NOs. 21, 23, 25, 27, 29, 31, 33, or 35.

Alternatively, α-ketoglutarate can be pushed towards the succinate pathway. α-ketoglutarate can be converted to succinyl CoA through the use of α-ketoglutarate dehydrogenase complex. Succinyl CoA can be converted to succinate through the use of succinyl-CoA synthase. Once succinate is formed, the succinate can be lead towards the 1,4-BDO pathway.

Succinate can be converted into a multicarbon product such as 1,4-BDO. In some cases, when the microorganism utilizes the succinate to 1,4-BDO pathway, the microorganism can comprise succinyl CoA synthease beta subunit (SucC), which is an enzyme that is capable of converting succinate to succinyl CoA. In some cases, the microorganism can comprise a succinyl CoA synthease alpha subunit (SucD), which is an enzyme that converts succinyl CoA to succinate semialdehyde. In some cases, the microorganism can comprise a 4-hydroxybutyrate dehydrogenase (4hbD), which is an enzyme that converts succinate semialdehyde to γ-hydroxybutyrate (GHB). In some cases, the microorganism can comprise a 4-hydroxybutyrate CoA transferase (cat2), which is an enzyme that converts γ-hydroxybutyrate (GHB) to 4-hydroxybutyryl CoA. In some cases, the microorganism can comprise an aldehyde dehydrogenase (ald), which is an enzyme that converts 4-hydroxybutyryl CoA to 4-hydroxybutyraldchyde. In some cases, the microorganism can comprise an alcohol dehydrogenase (adh), which is an enzyme that converts 4-hydroxybutyraldehyde to 1,4-BDO. One or more of these enzymes can be heterologous to the microorganism. Additionally, one or more of these enzymes can be endogenous to the microorganism.

Described here are microorganisms used to make 1,4-BDO from a C1 carbon (e.g., methane). In some cases, the microorganism herein can be transformed with a gene encoding for one or more of the following: i) succinyl CoA synthease beta subunit (sucC); ii) succinyl CoA synthease alpha subunit (sucD); iii) 4-hydroxybutyrate dehydrogenase (4hbD); iv) 4-hydroxybutyrate CoA transferase (Cat2); (v) aldehyde dehydrogenase (Ald); (vi) alcohol dehydrogenase (adh); or (vii) any combination thereof. These genes can be heterologous to the microorganism. The genes can also encode for an enzyme that can conduct the chemical conversions as described above. For example, the aldehyde dehydrogenase (ald) can be an enzyme that converts 4-hydroxybutyryl CoA to 4-hydroxybutyraldehyde, whereas the alcohol dehydrogenase (adh) can be an enzyme that converts 4-hydroxybutyraldchyde to 1,4-BDO.

When a succinyl CoA synthease beta subunit (SucC) is used the succinyl CoA synthease beta subunit can be from a bacteria (e.g., a gram negative bacterium), such as from the genus Escherichia. For example, the succinyl CoA synthease beta subunit can be from the species Escherichia coli.

The succinyl CoA synthease beta subunit can comprise an amino acid sequence that is substantially similar to SEQ ID NO. 37. For example, the succinyl CoA synthease beta subunit can comprise an amino acid sequence that is at least 60% identical to SEQ ID NO. 37. For example, the succinyl CoA synthease beta subunit can comprise an amino acid sequence that is at least 65% identical to SEQ ID NO. 37. In some cases, the succinyl CoA synthease beta subunit can comprise an amino acid sequence that is at least 70% identical to SEQ ID NO. 37. For example, the succinyl CoA synthease beta subunit can comprise an amino acid sequence that is at least 75% identical to SEQ ID NO. 37. In some cases, the succinyl CoA synthease beta subunit can comprise an amino acid sequence that is at least 80% identical to SEQ ID NO. 37. For example, the succinyl CoA synthease beta subunit can comprise an amino acid sequence that is at least 85% identical to SEQ ID NO. 37. In some cases, the succinyl CoA synthease beta subunit can comprise an amino acid sequence that is at least 90% identical to SEQ ID NO. 37. In some cases, the succinyl CoA synthease beta subunit can comprise an amino acid sequence that is at least 91% identical to SEQ ID NO. 37. In some cases, the succinyl CoA synthease beta subunit can comprise an amino acid sequence that is at least 92% identical to SEQ ID NO. 37. In some cases, the succinyl CoA synthease beta subunit can comprise an amino acid sequence that is at least 93% identical to SEQ ID NO. 37. In some cases, the succinyl CoA synthease beta subunit can comprise an amino acid sequence that is at least 94% identical to SEQ ID NO. 37. In some cases, the succinyl CoA synthease beta subunit can comprise an amino acid sequence that is at least 95% identical to SEQ ID NO. 37. In some cases, the succinyl CoA synthease beta subunit can comprise an amino acid sequence that is at least 96% identical to SEQ ID NO. 37. In some cases, the succinyl CoA synthease beta subunit can comprise an amino acid sequence that is at least 97% identical to SEQ ID NO. 37. In some cases, the succinyl CoA synthease beta subunit can comprise an amino acid sequence that is at least 98% identical to SEQ ID NO. 37. In some cases, the succinyl CoA synthease beta subunit can comprise an amino acid sequence that is at least 99% identical to SEQ ID NO. 37. In some cases, the succinyl CoA synthease beta subunit can comprise an amino acid sequence that is SEQ ID NO. 37.

When a succinyl CoA synthease alpha subunit (SucD) is used the succinyl CoA synthease alpha subunit can be from a bacteria (e.g., a gram negative or gram positive bacterium), such as from the genus Escherichia, Clostridium, or Porphyromonas. For example, the succinyl CoA synthease alpha subunit can be from the species Escherichia coli, Clostridium kluyveri, Porphyromonas gingivalis.

The succinyl CoA synthease alpha subunit (sucD) can comprise an amino acid sequence that is substantially similar to any one of SEQ ID NOs. 39, 41, or 43. For example, the succinyl CoA synthease alpha subunit can comprise an amino acid sequence that is at least 60% identical to any one of SEQ ID NOs. 39, 41, or 43. In some cases, the succinyl CoA synthease alpha subunit can comprise an amino acid sequence that is at least 65% identical to any one of SEQ ID NOs. 39, 41, or 43. In some cases, the succinyl CoA synthease alpha subunit can comprise an amino acid sequence that is at least 70% identical to any one of SEQ ID NOs. 39, 41, or 43. For example, the succinyl CoA synthease alpha subunit can comprise an amino acid sequence that is at least 75% identical to any one of SEQ ID NOs. 39, 41, or 43. In some cases, the succinyl CoA synthease alpha subunit can comprise an amino acid sequence that is at least 80% identical to any one of SEQ ID NOs. 39, 41, or 43. In some cases, the succinyl CoA synthease alpha subunit can comprise an amino acid sequence that is at least 85% identical to any one of SEQ ID NOs. 39, 41, or 43. In some cases, the succinyl CoA synthease alpha subunit can comprise an amino acid sequence that is at least 90% identical to any one of SEQ ID NOs. 39, 41, or 43. In some cases, the succinyl CoA synthease alpha subunit can comprise an amino acid sequence that is at least 91% identical to any one of SEQ ID NOs. 39, 41, or 43. In some cases, the succinyl CoA synthease alpha subunit can comprise an amino acid sequence that is at least 92% identical to any one of SEQ ID NOs. 39, 41, or 43. In some cases, the succinyl CoA synthease alpha subunit can comprise an amino acid sequence that is at least 93% identical to any one of SEQ ID NOs. 39, 41, or 43. In some cases, the succinyl CoA synthease alpha subunit can comprise an amino acid sequence that is at least 94% identical to any one of SEQ ID NOs. 39, 41, or 43. In some cases, the succinyl CoA synthease alpha subunit can comprise an amino acid sequence that is at least 95% identical to any one of SEQ ID NOs. 39, 41, or 43. In some cases, the succinyl CoA synthease alpha subunit can comprise an amino acid sequence that is at least 96% identical to any one of SEQ ID NOs. 39, 41, or 43. In some cases, the succinyl CoA synthease alpha subunit can comprise an amino acid sequence that is at least 97% identical to any one of SEQ ID NOs. 39, 41, or 43. In some cases, the succinyl CoA synthease alpha subunit can comprise an amino acid sequence that is at least 98% identical to any one of SEQ ID NOs. 39, 41, or 43. In some cases, the succinyl CoA synthease alpha subunit can comprise an amino acid sequence that is at least 99% identical to any one of SEQ ID NOs. 39, 41, or 43. In some cases, the succinyl CoA synthease alpha subunit can comprise an amino acid sequence that is any one of SEQ ID NOs. 39, 41, or 43.

The remaining four enzymes of the succinate to 1,4-BDO pathway are the same enzymes as the final four α-ketoglutarate to 1,4-BDO pathway enzymes. Therefore, when 4-hydroxybutyrate dehydrogenase (4hbD), 4-hydroxybutyrate CoA transferase (cat2), aldehyde dehydrogenase (ald), and/or alcohol dehydrogenase (adh) are desired, the respective enzymes disclosed for the α-ketoglutarate to 1,4-BDO pathway can be used.

Additionally, in some cases, the pathway to 1,4-BDO can be pushed from oxaloacetate/malyl-CoA to succinate, and then succinate to 1,4-BDO. In these cases, a fumarate hydratase (fum) and/or a fumarate reductase (frd) can be used.

When a fumarate hydratase (fum) is used the fumarate hydratase can be from a bacteria (e.g., a bacterium that is gram positive or gram negative), such as from the genus Methylococcus, Escherichia and/or Mycobacterium. For example, a fumarate hydratase (fum) can be from the species Methylococcus capsulatus, Escherichia coli, and/or Mycobacterium tuberculosis.

The fumarate hydratase (fum) can comprise an amino acid sequence that is substantially similar to any one of SEQ ID NOs. 45, 47, or 49. For example, the fumarate hydratase can comprise an amino acid sequence that is at least 60% identical to any one of SEQ ID NOs. 45, 47, or 49. For example, the fumarate hydratase can comprise an amino acid sequence that is at least 65% identical to any one of SEQ ID NOs. 45, 47, or 49. In some cases, the fumarate hydratase can comprise an amino acid sequence that is at least 70% identical to any one of SEQ ID NOs. 45, 47, or 49. For example, the fumarate hydratase can comprise an amino acid sequence that is at least 75% identical to any one of SEQ ID NOs. 45, 47, or 49. In some cases, the fumarate hydratase can comprise an amino acid sequence that is at least 80% identical to any one of SEQ ID NOs. 45, 47, or 49. For example, the fumarate hydratase can comprise an amino acid sequence that is at least 85% identical to any one of SEQ ID NOs. 45, 47, or 49. In some cases, the fumarate hydratase can comprise an amino acid sequence that is at least 90% identical to any one of SEQ ID NOs. 45, 47, or 49. In some cases, the fumarate hydratase can comprise an amino acid sequence that is at least 91% identical to any one of SEQ ID NOs. 45, 47, or 49. In some cases, the fumarate hydratase can comprise an amino acid sequence that is at least 92% identical to any one of SEQ ID NOs. 45, 47, or 49. In some cases, the fumarate hydratase can comprise an amino acid sequence that is at least 93% identical to any one of SEQ ID NOs. 45, 47, or 49. In some cases, the fumarate hydratase can comprise an amino acid sequence that is at least 94% identical to any one of SEQ ID NOs. 45, 47, or 49. In some cases, the fumarate hydratase can comprise an amino acid sequence that is at least 95% identical to any one of SEQ ID NOs. 45, 47, or 49. In some cases, the fumarate hydratase can comprise an amino acid sequence that is at least 96% identical to any one of SEQ ID NOs. 45, 47, or 49. In some cases, the fumarate hydratase can comprise an amino acid sequence that is at least 97% identical to any one of SEQ ID NOs. 45, 47, or 49. In some cases, the fumarate hydratase can comprise an amino acid sequence that is at least 98% identical to any one of SEQ ID NOs. 45, 47, or 49. In some cases, the fumarate hydratase can comprise an amino acid sequence that is at least 99% identical to any one of SEQ ID NOs. 45, 47, or 49. In some cases, the fumarate hydratase can comprise an amino acid sequence that is any one of SEQ ID NOs. 45, 47, or 49.

When a fumarate reductase (frd) is used the fumarate reductase can be from a bacteria (e.g., a bacterium that is gram negative), such as from the genus Escherichia. For example, a fumarate reductase (frd) can be from the species Escherichia coli.

The fumarate reductase (frd) can comprise an amino acid sequence that is substantially similar to any one of SEQ ID NOs. 51, 53, 55, or 57. For example, the fumarate reductase can comprise an amino acid sequence that is at least 60% identical to any one of SEQ ID NOs. 51, 53, 55, or 57. For example, the fumarate reductase can comprise an amino acid sequence that is at least 65% identical to any one of SEQ ID NOs. 51, 53, 55, or 57. In some cases, the fumarate reductase can comprise an amino acid sequence that is at least 70% identical to any one of SEQ ID NOs. 51, 53, 55, or 57. For example, the fumarate reductase can comprise an amino acid sequence that is at least 75% identical to any one of SEQ ID NOs. 51, 53, 55, or 57. In some cases, the fumarate reductase can comprise an amino acid sequence that is at least 80% identical to any one of SEQ ID NOs. 51, 53, 55, or 57. For example, the fumarate reductase can comprise an amino acid sequence that is at least 85% identical to any one of SEQ ID NOs. 51, 53, 55, or 57. In some cases, the fumarate reductase can comprise an amino acid sequence that is at least 90% identical to any one of SEQ ID NOs. 51, 53, 55, or 57. In some cases, the fumarate reductase can comprise an amino acid sequence that is at least 91% identical to any one of SEQ ID NOs. 51, 53, 55, or 57. In some cases, the fumarate reductase can comprise an amino acid sequence that is at least 92% identical to any one of SEQ ID NOs. 51, 53, 55, or 57. In some cases, the fumarate reductase can comprise an amino acid sequence that is at least 93% identical to any one of SEQ ID NOs. 51, 53, 55, or 57. In some cases, the fumarate reductase can comprise an amino acid sequence that is at least 94% identical to any one of SEQ ID NOs. 51, 53, 55, or 57. In some cases, the fumarate reductase can comprise an amino acid sequence that is at least 95% identical to any one of SEQ ID NOs. 51, 53, 55, or 57. In some cases, the fumarate reductase can comprise an amino acid sequence that is at least 96% identical to any one of SEQ ID NOs. 51, 53, 55, or 57. In some cases, the fumarate reductase can comprise an amino acid sequence that is at least 97% identical to any one of SEQ ID NOs. 51, 53, 55, or 57. In some cases, the fumarate reductase can comprise an amino acid sequence that is at least 98% identical to any one of SEQ ID NOs. 51, 53, 55, or 57. In some cases, the fumarate reductase can comprise an amino acid sequence that is at least 99% identical to any one of SEQ ID NOs. 51, 53, 55, or 57. In some cases, the fumarate reductase can comprise an amino acid sequence that is any one of SEQ ID NOs. 51, 53, 55, or 57.

Additional enzymes can be placed inside the microorganism in order to make other desired end products by fermentation.

The amino acid sequence can also be optimized based on the microorganism in which the enzymes will be expressed. In other words, conservative amino acids substitutions can be made based on whether the respective microorganism typically uses a specific amino acid or how much of that particular amino acid is available for use within the microorganism.

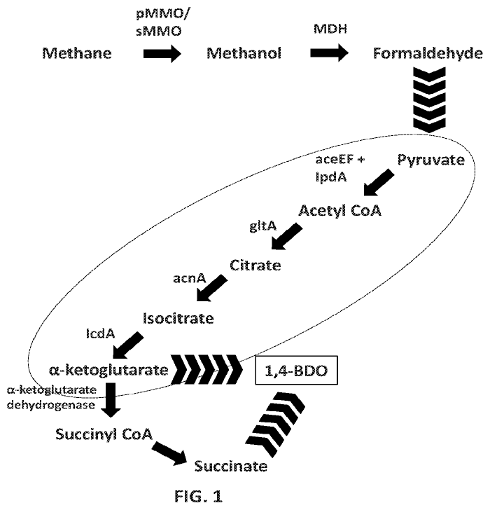

Additionally, in some cases, two or more enzymes that catalyze consecutive reactions can be used within the microorganism. For example, the two enzymes that catalyze consecution reactions can be α-ketoglutarate dehydrogenase and 4-hyrobutyrate dehydrogenase. Any combination of consecutive enzymes can be used and can be found in FIGS. 1, 2, 3, and 7. In some cases, three or more enzymes that catalyze consecutive reactions can be used within the microorganism. In some cases, four or more enzymes that catalyze consecutive reactions can be used within the microorganism. In some cases, five or more enzymes that catalyze consecutive reactions can be used within the microorganism. In some cases, six or more enzymes that catalyze consecutive reactions can be used within the microorganism. In some cases, seven or more enzymes that catalyze consecutive reactions can be used within the microorganism. In some cases, eight or more enzymes that catalyze consecutive reactions can be used within the microorganism. In some cases, nine or more enzymes that catalyze consecutive reactions can be used within the microorganism. In some cases, ten or more enzymes that catalyze consecutive reactions can be used within the microorganism.

### Vectors

Polynucleotide constructs prepared for introduction into a prokaryotic or eukaryotic host may typically, but not always, comprise a replication system (i.e. vector) recognized by the host, including the intended polynucleotide fragment encoding the desired polypeptide, and can, but not necessarily, also include transcription and translational initiation regulatory sequences operably linked to the polypeptide-encoding segment. Expression systems (such as expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides may also be included where appropriate, preferably from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N. Y. 1989; and Ausubel, et al., Current Protocols in Molecular Biology, Greene Publishing, Co. N.Y, 1995).

The manipulation of polynucleotides that encode the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector may be selected to accommodate a polynucleotide encoding a protein of a desired size. Following recombinant modification of a selected vector, a suitable host cell (e.g., the microorganisms described herein) is transfected or transformed with the vector. Each vector contains various functional components, which generally include a cloning site, an origin of replication and at least one selectable marker gene. A vector may additionally possess one or more of the following elements: an enhancer, promoter, and transcription termination and/or other signal sequences. Such sequence elements may be optimized for the selected host species. Such sequence elements may be positioned in the vicinity of the cloning site, such that they are operatively linked to the gene encoding a preselected enzyme.

Vectors, including cloning and expression vectors, may contain nucleic acid sequences that enable the vector to replicate in one or more selected microorganisms. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV40, adenovirus) are useful for cloning vectors.

A cloning or expression vector may contain a selection gene (also referred to as a selectable marker). This gene encodes a protein necessary for the survival or growth of transformed microorganisms in a selective culture medium. Microorganisms not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli. An E. coli-selectable marker, for example, the B-lactamase gene that confers resistance to the antibiotic ampicillin, may be of use. These selectable markers can be obtained from E. coli plasmids, such as pBR322 or a pUC plasmid such as pUC18 or pUC19, or pUC119.

### Promoters

Vectors may contain a promoter that is recognized by the host microorganism. The promoter may be operably linked to a coding sequence of interest. Such a promoter may be inducible or constitutive. Polynucleotides are operably linked when the polynucleotides are in a relationship permitting them to function in their intended manner.

Different promoters can be used to drive the expression of the genes. For example, if temporary gene expression (i.e., non-constitutively expressed) is desired, expression can be driven by inducible promoters.

In some cases, some of the genes disclosed can be expressed temporarily. In other words, the genes are not constitutively expressed. The expression of the genes can be driven by inducible or repressible promoters. For example, the inducible or repressible promoters that can be used include but are not limited to: (a) sugars such as arabinose and lactose (or non metabolizable analogs, e.g., isopropyl β-D-1-thiogalactopyranoside (IPTG)); (b) metals such as lanthanum (or other rare earth metals), copper, calcium; (c) temperature; (d) Nitrogen-source; (e) oxygen; (f) cell state (growth or stationary); (g) metabolites such as phosphate; (h) CRISPRi; (i) jun; (j) fos, (k) metallothionein and/or (1) heat shock. These promoters can be used in a methanotroph systems. For example, one example of an inducible promoter that can be used within the methanotrophs is a pBAD or a pMxaF promoter.

Constitutively expressed promoters can also be used in the vector systems herein. For example, the expression of some of the genes disclosed throughout can be controlled by constitutively active promoters. For examples, the promoters that can be used include but are not limited to p.Bba.J23111, J23111, and J23100.

Promoters suitable for use with prokaryotic hosts may include, for example, the a-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Promoters for use in bacterial systems will also generally contain a Shine-Dalgarno sequence operably linked to the coding sequence.

Generally, a strong promoter may be employed to provide for high level transcription and expression of the desired product.

One or more promoters of a transcription unit can be an inducible promoter. For example, a GFP can be expressed from a constitutive promoter while an inducible promoter drives transcription of a gene coding for one or more enzymes as disclosed herein and/or the amplifiable selectable marker.

Some vectors may contain prokaryotic sequences that facilitate the propagation of the vector in bacteria. Thus, the vectors may have other components such as an origin of replication (e.g., a nucleic acid sequence that enables the vector to replicate in one or more selected microorganisms), antibiotic resistance genes for selection in bacteria, and/or an amber stop codon which can permit translation to read through the codon. Additional selectable gene(s) may also be incorporated. Generally, in cloning vectors the origin of replication is one that enables the vector to replicate independently of the host chromosomal DNA, and includes origins of replication or autonomously replicating sequences. Such sequences can include the ColEl origin of replication in bacteria or other known sequences.

### Genes

The vectors can comprise a nucleic acid sequence of one or more enzymes that are capable of catalyzing one or more of the following reactions: i) methane to methanol; ii) methanol to formaldehyde; iii) formaldehyde to pyruvate; iv) pyruvate to acetyl CoA; v) acetyl CoA to citrate; vi) citrate to isocitrate; vii) isocitrate to α-ketoglutarate; viii) α-ketoglutarate to succinate CoA; ix) succinate CoA to succinate; x) oxaloacetate and/or malyl-CoA to L-malate; xi) L-malate to fumarate; and/or xii) fumarate to succinate.

In some instances, the vector can comprise one or more of the following genes from the α-ketoglutarate to 1,4-BDO pathway: i) α-ketoglutarate decarboxylase (kgd); ii) 4-hydroxybutyrate dehydrogenase (4hbD); iii) 4-hydroxybutyrate CoA transferase (Cat2); (iv) aldehyde dehydrogenase (Ald); (v) alcohol dehydrogenase (adh); or (vi) any combination thereof.

In some cases, the vector can comprise one or more of the following genes from the succinate to 1,4-BDO pathway: i) succinyl CoA synthease beta subunit (sucC); ii) succinyl CoA synthease alpha subunit (sucD); iii) 4-hydroxybutyrate dehydrogenase (4hbD); iv) 4-hydroxybutyrate CoA transferase (Cat2); (v) aldehyde dehydrogenase (Ald); (vi) alcohol dehydrogenase (adh); or (vii) any combination thereof.

The vector in some cases can comprise a fumarate hydratase and/or fumarate reductase.

One or more of the genes can be heterologous to the microorganism in which the vector is contacted with (and eventually transformed with).

It is also contemplated that any and all genes disclosed herein can be overexpressed. For example, when a microorganism or vector comprises a gene (e.g., a heterologous gene), the gene can be overexpressed. This is done typically but using a promoter that is highly expressed or inserting multiple copies of the gene.

When an α-ketoglutarate decarboxylase (kgd) gene is used the α-ketoglutarate decarboxylase gene can be from a bacteria (e.g., a gram positive bacterium or a bacterium that is neither gram positive or gram negative), such as from the genus Mycobacterium, Corynebacterium and/or Rhodococcus. For example, an α-ketoglutarate decarboxylase (kgd) gene can be from the species Mycobacterium bovis, Mycobacterium tuberculosis, Corynebacterium terpenotabidum, and/or Rhodococcus jostii.

The α-ketoglutarate decarboxylase (kgd) can comprise a nucleic acid sequence that is substantially similar to any one of SEQ ID NOs. 2, 4, 6, or 8. For example, the α-ketoglutarate decarboxylase can comprise a nucleic acid sequence that is at least 60% identical to any one of SEQ ID NOs. 2, 4, 6, or 8. In some cases, the α-ketoglutarate decarboxylase can comprise a nucleic acid sequence that is at least 65% identical to any one of SEQ ID NOs. 2, 4, 6, or 8. In some cases, the a-ketoglutarate decarboxylase can comprise a nucleic acid sequence that is at least 70% identical to any one of SEQ ID NOs. 2, 4, 6, or 8. In some cases, the α-ketoglutarate decarboxylase can comprise a nucleic acid sequence that is at least 75% identical to any one of SEQ ID NOs. 2, 4, 6, or 8. In some cases, the α-ketoglutarate decarboxylase can comprise a nucleic acid sequence that is at least 80% identical to any one of SEQ ID NOs. 2, 4, 6, or 8. In some cases, the α-ketoglutarate decarboxylase can comprise a nucleic acid sequence that is at least 85% identical to any one of SEQ ID NOs. 2, 4, 6, or 8. In some cases, the α-ketoglutarate decarboxylase can comprise a nucleic acid sequence that is at least 90% identical to any one of SEQ ID NOs. 2, 4, 6, or 8. In some cases, the α-ketoglutarate decarboxylase can comprise a nucleic acid sequence that is at least 91% identical to any one of SEQ ID NOs. 2, 4, 6, or 8. In some cases, the α-ketoglutarate decarboxylase can comprise a nucleic acid sequence that is at least 92% identical to any one of SEQ ID NOs. 2, 4, 6, or 8. In some cases, the α-ketoglutarate decarboxylase can comprise a nucleic acid sequence that is at least 93% identical to any one of SEQ ID NOs. 2, 4, 6, or 8. In some cases, the α-ketoglutarate decarboxylase can comprise a nucleic acid sequence that is at least 94% identical to any one of SEQ ID NOs. 2, 4, 6, or 8. In some cases, the α-ketoglutarate decarboxylase can comprise a nucleic acid sequence that is at least 95% identical to any one of SEQ ID NOs. 2, 4, 6, or 8. In some cases, the α-ketoglutarate decarboxylase can comprise a nucleic acid sequence that is at least 96% identical to any one of SEQ ID NOs. 2, 4, 6, or 8. In some cases, the α-ketoglutarate decarboxylase can comprise a nucleic acid sequence that is at least 97% identical to any one of SEQ ID NOs. 2, 4, 6, or 8. In some cases, the α-ketoglutarate decarboxylase can comprise a nucleic acid sequence that is at least 98% identical to any one of SEQ ID NOs. 2, 4, 6, or 8. In some cases, the α-ketoglutarate decarboxylase can comprise a nucleic acid sequence that is at least 99% identical to any one of SEQ ID NOs. 2, 4, 6, or 8. In some cases, the α-ketoglutarate decarboxylase can comprise a nucleic acid sequence that is any one of SEQ ID NOs. 2, 4, 6, or 8.

When a 4-hydroxybutyrate dehydrogenase (4hbD) gene is desired the 4-hydroxybutyrate dehydrogenase gene can be from a bacterium (e.g., a gram negative or positive bacterium), such as from the genus Escherichia, Porphyromonas, and/or Clostridium. For example, a 4-hydroxybutyrate dehydrogenase gene can be from the species Escherichia coli, Porphyromonas gingivalis, and/or Clostridium kluyveri.

The 4-hydroxybutyrate dehydrogenase gene can comprise a nucleic acid sequence that is substantially similar to SEQ ID NO. 12 or 14. For example, the 4-hydroxybutyrate dehydrogenase gene can comprise a nucleic acid sequence that is at least 60% identical to SEQ ID NO. 12 or 14. For example, the 4-hydroxybutyrate dehydrogenase gene can comprise a nucleic acid sequence that is at least 65% identical to SEQ ID NO. 12 or 14. In some cases, the 4-hydroxybutyrate dehydrogenase gene can comprise a nucleic acid sequence that is at least 70% identical to SEQ ID NO. 12 or 14. For example, the 4-hydroxybutyrate dehydrogenase gene can comprise a nucleic acid sequence that is at least 75% identical to SEQ ID NO. 12 or 14. In some cases, the 4-hydroxybutyrate dehydrogenase gene can comprise a nucleic acid sequence that is at least 80% identical to SEQ ID NO. 12 or 14. For example, the 4-hydroxybutyrate dehydrogenase gene can comprise a nucleic acid sequence that is at least 85% identical to SEQ ID NO. 12 or 14. In some cases, the 4-hydroxybutyrate dehydrogenase gene can comprise a nucleic acid sequence that is at least 90% identical to SEQ ID NO. 12 or 14. In some cases, the 4-hydroxybutyrate dehydrogenase gene can comprise a nucleic acid sequence that is at least 91% identical to SEQ ID NO. 12 or 14. In some cases, the 4-hydroxybutyrate dehydrogenase gene can comprise a nucleic acid sequence that is at least 92% identical to SEQ ID NO. 12 or 14. In some cases, the 4-hydroxybutyrate dehydrogenase gene can comprise a nucleic acid sequence that is at least 93% identical to SEQ ID NO. 12 or 14. In some cases, the 4-hydroxybutyrate dehydrogenase gene can comprise a nucleic acid sequence that is at least 94% identical to SEQ ID NO. 12 or 14. In some cases, the 4-hydroxybutyrate dehydrogenase gene can comprise a nucleic acid sequence that is at least 95% identical to SEQ ID NO. 12 or 14. In some cases, the 4-hydroxybutyrate dehydrogenase gene can comprise a nucleic acid sequence that is at least 96% identical to SEQ ID NO. 12 or 14. In some cases, the 4-hydroxybutyrate dehydrogenase gene can comprise a nucleic acid sequence that is at least 97% identical to SEQ ID NO. 12 or 14. In some cases, the 4-hydroxybutyrate dehydrogenase gene can comprise a nucleic acid sequence that is at least 98% identical to SEQ ID NO. 12 or 14. In some cases, the 4-hydroxybutyrate dehydrogenase gene can comprise a nucleic acid sequence that is at least 99% identical to SEQ ID NO. 12 or 14. In some cases, the 4-hydroxybutyrate dehydrogenase gene can comprise a nucleic acid sequence that is SEQ ID NO. 12 or 14.

When a 4-hydroxybutyrate CoA transferase (cat2) gene is desired the 4-hydroxybutyrate CoA transferase gene can be from a bacterium (e.g., a gram negative or positive bacterium), such as from the genus Porphyromonas, and/or Clostridium. For example, a 4-hydroxybutyrate CoA transferase gene can be from the species Porphyromonas gingivalis and/or Clostridium acetobutylicum.

The 4-hydroxybutyrate CoA transferase gene can comprise a nucleic acid sequence that is substantially similar to any one of SEQ ID NOs. 16, 18, or 20. For example, the 4-hydroxybutyrate CoA transferase gene can comprise a nucleic acid sequence that is at least 60% identical to any one of SEQ ID NOs. 16, 18, or 20. For example, the 4-hydroxybutyrate CoA transferase gene can comprise a nucleic acid sequence that is at least 65% identical to any one of SEQ ID NOs. 16, 18, or 20. In some cases, the 4-hydroxybutyrate CoA transferase gene can comprise a nucleic acid sequence that is at least 70% identical to any one of SEQ ID NOs. 16, 18, or 20. For example, the 4-hydroxybutyrate CoA transferase gene can comprise a nucleic acid sequence that is at least 75% identical to any one of SEQ ID NOs. 16, 18, or 20. In some cases, the 4-hydroxybutyrate CoA transferase gene can comprise a nucleic acid sequence that is at least 80% identical to any one of SEQ ID NOs. 16, 18, or 20. For example, the 4-hydroxybutyrate CoA transferase gene can comprise a nucleic acid sequence that is at least 85% identical to any one of SEQ ID NOs. 16, 18, or 20. In some cases, the 4-hydroxybutyrate CoA transferase gene can comprise a nucleic acid sequence that is at least 90% identical to any one of SEQ ID NOs. 16, 18, or 20. In some cases, the 4-hydroxybutyrate CoA transferase gene can comprise a nucleic acid sequence that is at least 91% identical to any one of SEQ ID NOs. 16, 18, or 20. In some cases, the 4-hydroxybutyrate CoA transferase gene can comprise a nucleic acid sequence that is at least 92% identical to any one of SEQ ID NOs. 16, 18, or 20. In some cases, the 4-hydroxybutyrate CoA transferase gene can comprise a nucleic acid sequence that is at least 93% identical to any one of SEQ ID NOs. 16, 18, or 20. In some cases, the 4-hydroxybutyrate CoA transferase gene can comprise a nucleic acid sequence that is at least 94% identical to any one of SEQ ID NOs. 16, 18, or 20. In some cases, the 4-hydroxybutyrate CoA transferase gene can comprise a nucleic acid sequence that is at least 95% identical to any one of SEQ ID NOs. 16, 18, or 20. In some cases, the 4-hydroxybutyrate CoA transferase gene can comprise a nucleic acid sequence that is at least 96% identical to any one of SEQ ID NOs. 16, 18, or 20. In some cases, the 4-hydroxybutyrate CoA transferase gene can comprise a nucleic acid sequence that is at least 97% identical to any one of SEQ ID NOs. 16, 18, or 20. In some cases, the 4-hydroxybutyrate CoA transferase gene can comprise a nucleic acid sequence that is at least 98% identical to any one of SEQ ID NOs. 16, 18, or 20. In some cases, the 4-hydroxybutyrate CoA transferase gene can comprise a nucleic acid sequence that is at least 99% identical to any one of SEQ ID NOs. 16, 18, or 20. In some cases, the 4-hydroxybutyrate CoA transferase gene can comprise a nucleic acid sequence that is any one of SEQ ID NOs. 16, 18, or 20.

When an aldehyde dehydrogenase (ald) gene and/or an alcohol dehydrogenase (adh) gene is desired the aldehyde dehydrogenase gene and/or alcohol dehydrogenase gene can be from a bacterium (e.g., a gram negative or positive bacterium), such as from the genus Escherichia, Acinetobacter, Porphyromonas, and/or Clostridium, or from a yeast such as from the genus Saccharomyces. For example, an aldehyde dehydrogenase gene and/or alcohol dehydrogenase gene can be from the species Escherichia coli, Acinetobacter baylyi, Porphyromonas gingivalis, Clostridium acetobutylicum and/or Saccharomyces cerevisiae. In some cases, more than one aldehyde dehydrogenase gene and/or alcohol dehydrogenase gene can be used.

The aldehyde dehydrogenase gene and/or alcohol dehydrogenase gene can comprise a nucleic acid sequence that is substantially similar to any one of SEQ ID NOs. 22, 24, 26, 28, 30, 32, 34, or 36. For example, the aldehyde dehydrogenase gene and/or alcohol dehydrogenase gene can comprise a nucleic acid sequence that is at least 60% identical to any one of SEQ ID NOs. 22, 24, 26, 28, 30, 32, 34, or 36. For example, the aldehyde dehydrogenase gene and/or alcohol dehydrogenase gene can comprise a nucleic acid sequence that is at least 65% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 70% identical to any one of SEQ ID NOs. 22, 24, 26, 28, 30, 32, 34, or 36. For example, the aldehyde dehydrogenase gene and/or alcohol dehydrogenase gene can comprise a nucleic acid sequence that is at least 75% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 80% identical to any one of SEQ ID NOs. 22, 24, 26, 28, 30, 32, 34, or 36. For example, the aldehyde dehydrogenase gene and/or alcohol dehydrogenase gene can comprise a nucleic acid sequence that is at least 85% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 90% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 91% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 92% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 93% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 94% identical to any one of SEQ ID NOS. 22, 24, 26, 28, 30, 32, 34, or 36. In some cases, the aldehyde dehydrogenase gene and/or alcohol dehydrogenase gene can comprise a nucleic acid sequence that is at least 95% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 96% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 97% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 98% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is at least 99% identical to any one of SEQ ID NOs. 22, 24, 26, 28, 30, 32, 34, or 36. In some cases, the aldehyde dehydrogenase gene and/or alcohol dehydrogenase gene can comprise a nucleic acid sequence that is any one of SEQ ID NOs. 22, 24, 26, 28, 30, 32, 34, or 36.

When a succinyl CoA synthease beta subunit (sucC) gene is used the succinyl CoA synthease beta subunit gene can be from a bacteria (e.g., a gram negative bacterium), such as from the genus Escherichia. For example, the succinyl CoA synthease beta subunit gene can be from the species Escherichia coli.

The succinyl CoA synthease beta subunit gene can comprise a nucleic acid sequence that is substantially similar to SEQ ID NO. 38. For example, the succinyl CoA synthease beta subunit gene can comprise a nucleic acid sequence that is at least 60% identical to SEQ ID NO. 38. For example, the succinyl CoA synthease beta subunit gene can comprise a nucleic acid sequence that is at least 65% identical to SEQ ID NO. 38. In some cases, the succinyl CoA synthease beta subunit gene can comprise a nucleic acid sequence that is at least 70% identical to SEQ ID NO. 38. For example, the succinyl CoA synthease beta subunit gene can comprise a nucleic acid sequence that is at least 75% identical to SEQ ID NO. 38. In some cases, the succinyl CoA synthease beta subunit gene can comprise a nucleic acid sequence that is at least 80% identical to SEQ ID NO. 38. For example, the succinyl CoA synthease beta subunit gene can comprise a nucleic acid sequence that is at least 85% identical to SEQ ID NO. 38. In some cases, the succinyl CoA synthease beta subunit gene can comprise a nucleic acid sequence that is at least 90% identical to SEQ ID NO. 38. In some cases, the succinyl CoA synthease beta subunit gene can comprise a nucleic acid sequence that is at least 91% identical to SEQ ID NO. 38. In some cases, the succinyl CoA synthease beta subunit gene can comprise a nucleic acid sequence that is at least 92% identical to SEQ ID NO. 38. In some cases, the succinyl CoA synthease beta subunit gene can comprise a nucleic acid sequence that is at least 93% identical to SEQ ID NO. 38. In some cases, the succinyl CoA synthease beta subunit gene can comprise a nucleic acid sequence that is at least 94% identical to SEQ ID NO. 38. In some cases, the succinyl CoA synthease beta subunit gene can comprise a nucleic acid sequence that is at least 95% identical to SEQ ID NO. 38. In some cases, the succinyl CoA synthease beta subunit gene can comprise a nucleic acid sequence that is at least 96% identical to SEQ ID NO. 38. In some cases, the succinyl CoA synthease beta subunit gene can comprise a nucleic acid sequence that is at least 97% identical to SEQ ID NO. 38. In some cases, the succinyl CoA synthease beta subunit gene can comprise a nucleic acid sequence that is at least 98% identical to SEQ ID NO. 38. In some cases, the succinyl CoA synthease beta subunit gene can comprise a nucleic acid sequence that is at least 99% identical to SEQ ID NO. 38. In some cases, the succinyl CoA synthease beta subunit gene can comprise a nucleic acid sequence that is SEQ ID NO. 38.

When a succinyl CoA synthease alpha subunit (sucD) gene is used the succinyl CoA synthease alpha subunit gene can be from a bacteria (e.g., a gram negative or gram positive bacterium), such as from the genus Escherichia, Clostridium, or Porphyromonas. For example, the succinyl CoA synthease alpha subunit gene can be from the species Escherichia coli, Clostridium kluyveri, Porphyromonas gingivalis.

The succinyl CoA synthease alpha subunit gene can comprise a nucleic acid sequence that is substantially similar to any one of SEQ ID NOs. 40, 43, or 44. For example, the succinyl CoA synthease alpha subunit gene can comprise a nucleic acid sequence that is at least 60% identical to any one of SEQ ID NOs. 40, 43, or 44. For example, the succinyl CoA synthease alpha subunit gene can comprise a nucleic acid sequence that is at least 65% identical to any one of SEQ ID NOs. 40, 43, or 44. In some cases, the succinyl CoA synthease alpha subunit gene can comprise a nucleic acid sequence that is at least 70% identical to any one of SEQ ID NOs. 40, 43, or 44. For example, the succinyl CoA synthease alpha subunit gene can comprise a nucleic acid sequence that is at least 75% identical to any one of SEQ ID NOs. 40, 43, or 44. In some cases, the succinyl CoA synthease alpha subunit gene can comprise a nucleic acid sequence that is at least 80% identical to any one of SEQ ID NOs. 40, 43, or 44. For example, the succinyl CoA synthease alpha subunit gene can comprise a nucleic acid sequence that is at least 85% identical to any one of SEQ ID NOs. 40, 43, or 44. In some cases, the succinyl CoA synthease alpha subunit gene can comprise a nucleic acid sequence that is at least 90% identical to any one of SEQ ID NOs. 40, 43, or 44. In some cases, the succinyl CoA synthease alpha subunit gene can comprise a nucleic acid sequence that is at least 91% identical to any one of SEQ ID NOs. 40, 43, or 44. In some cases, the succinyl CoA synthease alpha subunit gene can comprise a nucleic acid sequence that is at least 92% identical to any one of SEQ ID NOs. 40, 43, or 44. In some cases, the succinyl CoA synthease alpha subunit gene can comprise a nucleic acid sequence that is at least 93% identical to any one of SEQ ID NOs. 40, 43, or 44. In some cases, the succinyl CoA synthease alpha subunit gene can comprise a nucleic acid sequence that is at least 94% identical to any one of SEQ ID NOs. 40, 43, or 44. In some cases, the succinyl CoA synthease alpha subunit gene can comprise a nucleic acid sequence that is at least 95% identical to any one of SEQ ID NOs. 40, 43, or 44. In some cases, the succinyl CoA synthease alpha subunit gene can comprise a nucleic acid sequence that is at least 96% identical to any one of SEQ ID NOs. 40, 43, or 44. In some cases, the succinyl CoA synthease alpha subunit gene can comprise a nucleic acid sequence that is at least 97% identical to any one of SEQ ID NOs. 40, 43, or 44. In some cases, the succinyl CoA synthease alpha subunit gene can comprise a nucleic acid sequence that is at least 98% identical to any one of SEQ ID NOs. 40, 43, or 44. In some cases, the succinyl CoA synthease alpha subunit gene can comprise a nucleic acid sequence that is at least 99% identical to any one of SEQ ID NOs. 40, 43, or 44. In some cases, the succinyl CoA synthease alpha subunit gene can comprise a nucleic acid sequence that is any one of SEQ ID NOs. 40, 43, or 44.

The remaining four enzymes of the succinate to 1,4-BDO pathway are the same enzymes as the final four α-ketoglutarate to 1,4-BDO pathway enzymes. Therefore, when 4-hydroxybutyrate dehydrogenase gene, 4-hydroxybutyrate CoA transferase gene, aldehyde dehydrogenase gene, and/or alcohol dehydrogenase gene are desired, the respective genes disclosed for the α-ketoglutarate to 1,4-BDO pathway can be used.

Additionally, in some cases, the pathway to 1,4-BDO can be pushed from oxaloacetate/malyl-CoA to succinate, and then succinate to 1,4-BDO. In these cases, a fumarate hydratase (fum) and/or a fumarate reductase (frd) can be used.

When a fumarate hydratase (fum) gene is used the fumarate hydratase gene can be from a bacteria (e.g., a bacterium that is gram positive or gram negative), such as from the genus Methylococcus, Escherichia and/or Mycobacterium. For example, a fumarate hydratase (fum) gene can be from the species Methylococcus capsulatus, Escherichia coli, and/or Mycobacterium tuberculosis.

The fumarate hydratase (fum) gene can comprise a nucleic acid sequence that is substantially similar to any one of SEQ ID NOs. 46, 48, or 50. For example, the fumarate hydratase gene can comprise a nucleic acid sequence that is at least 60% identical to any one of SEQ ID NOs. 46, 48, or 50. For example, the fumarate hydratase gene can comprise a nucleic acid sequence that is at least 65% identical to any one of SEQ ID NOs. 46, 48, or 50. In some cases, the fumarate hydratase gene can comprise a nucleic acid sequence that is at least 70% identical to any one of SEQ ID NOs. 46, 48, or 50. For example, the fumarate hydratase gene can comprise a nucleic acid sequence that is at least 75% identical to any one of SEQ ID NOs. 46, 48, or 50. In some cases, the fumarate hydratase gene can comprise a nucleic acid sequence that is at least 80% identical to any one of SEQ ID NOs. 46, 48, or 50. For example, the fumarate hydratase gene can comprise a nucleic acid sequence that is at least 85% identical to any one of SEQ ID NOs. 46, 48, or 50. In some cases, the fumarate hydratase gene can comprise a nucleic acid sequence that is at least 90% identical to any one of SEQ ID NOs. 46, 48, or 50. In some cases, the fumarate hydratase gene can comprise a nucleic acid sequence that is at least 91% identical to any one of SEQ ID NOs. 46, 48, or 50. In some cases, the fumarate hydratase gene can comprise a nucleic acid sequence that is at least 92% identical to any one of SEQ ID NOs. 46, 48, or 50. In some cases, the fumarate hydratase gene can comprise a nucleic acid sequence that is at least 93% identical to any one of SEQ ID NOs. 46, 48, or 50. In some cases, the fumarate hydratase gene can comprise a nucleic acid sequence that is at least 94% identical to any one of SEQ ID NOs. 46, 48, or 50. In some cases, the fumarate hydratase gene can comprise a nucleic acid sequence that is at least 95% identical to any one of SEQ ID NOs. 46, 48, or 50. In some cases, the fumarate hydratase gene can comprise a nucleic acid sequence that is at least 96% identical to any one of SEQ ID NOs. 46, 48, or 50. In some cases, the fumarate hydratase gene can comprise a nucleic acid sequence that is at least 97% identical to any one of SEQ ID NOs. 46, 48, or 50. In some cases, the fumarate hydratase gene can comprise a nucleic acid sequence that is at least 98% identical to any one of SEQ ID NOs. 46, 48, or 50. In some cases, the fumarate hydratase gene can comprise a nucleic acid sequence that is at least 99% identical to any one of SEQ ID NOs. 46, 48, or 50. In some cases, the fumarate hydratase gene can comprise a nucleic acid sequence that is any one of SEQ ID NOs. 46, 48, or 50.

When a fumarate reductase (frd) gene is used the fumarate reductase gene can be from a bacteria (e.g., a bacterium that is gram negative), such as from the genus Escherichia. For example, a fumarate reductase (frd) gene can be from the species Escherichia coli.

The fumarate reductase (frd) gene can comprise a nucleic acid sequence that is substantially similar to any one of SEQ ID NOs. 52, 54, 56, or 58. For example, the fumarate reductase gene can comprise a nucleic acid sequence that is at least 60% identical to any one of SEQ ID NOs. 52, 54, 56, or 58. For example, the fumarate reductase gene can comprise a nucleic acid sequence that is at least 65% identical to any one of SEQ ID NOs. 52, 54, 56, or 58. In some cases, the fumarate reductase gene can comprise a nucleic acid sequence that is at least 70% identical to any one of SEQ ID NOS. 52, 54, 56, or 58. For example, the fumarate reductase gene can comprise a nucleic acid sequence that is at least 75% identical to any one of SEQ ID NOs. 52, 54, 56, or 58. In some cases, the fumarate reductase gene can comprise a nucleic acid sequence that is at least 80% identical to any one of SEQ ID NOs. 52, 54, 56, or 58. For example, the fumarate reductase gene can comprise a nucleic acid sequence that is at least 85% identical to any one of SEQ ID NOs. 52, 54, 56, or 58. In some cases, the fumarate reductase gene can comprise a nucleic acid sequence that is at least 90% identical to any one of SEQ ID NOs. 52, 54, 56, or 58. In some cases, the fumarate reductase gene can comprise a nucleic acid sequence that is at least 91% identical to any one of SEQ ID NOs. 52, 54, 56, or 58. In some cases, the fumarate reductase gene can comprise a nucleic acid sequence that is at least 92% identical to any one of SEQ ID NOs. 52, 54, 56, or 58. In some cases, the fumarate reductase gene can comprise a nucleic acid sequence that is at least 93% identical to any one of SEQ ID NOs. 52, 54, 56, or 58. In some cases, the fumarate reductase gene can comprise a nucleic acid sequence that is at least 94% identical to any one of SEQ ID NOs. 52, 54, 56, or 58. In some cases, the fumarate reductase gene can comprise a nucleic acid sequence that is at least 95% identical to any one of SEQ ID NOS. 52, 54, 56, or 58. In some cases, the fumarate reductase gene can comprise a nucleic acid sequence that is at least 96% identical to any one of SEQ ID NOs. 52, 54, 56, or 58. In some cases, the fumarate reductase gene can comprise a nucleic acid sequence that is at least 97% identical to any one of SEQ ID NOs. 52, 54, 56, or 58. In some cases, the fumarate reductase gene can comprise a nucleic acid sequence that is at least 98% identical to any one of SEQ ID NOs. 52, 54, 56, or 58. In some cases, the fumarate reductase gene can comprise a nucleic acid sequence that is at least 99% identical to any one of SEQ ID NOs. 52, 54, 56, or 58. In some cases, the fumarate reductase gene can comprise a nucleic acid sequence that is any one of SEQ ID NOs. 52, 54, 56, or 58.

Additional genes can be placed inside the microorganism in order to make other desired end products by fermentation.

The nucleotide sequence (or more specifically the codons that are encoded by the nucleotide sequences) can be optimized based on the microorganism in which the nucleotide sequences will be expressed. The nucleotide sequences can be codon optimized based on the amount of tRNA available within each individual microorganism. In other words, conservative codon substitutions can be made based on whether the respective microorganism typically uses a specific codon or how much of a particular tRNA is available within the microorganism.

Additionally, in some cases, genes encoding for two or more enzymes that catalyze consecutive reactions can be expressed within the microorganism. For example, two or more genes encoding for enzymes that catalyze consecution reactions can be an α-ketoglutarate dehydrogenase gene and a 4-hyrobutyrate dehydrogenase gene. Any combination of consecutive enzymes can be expressed and can be found in FIGS. 1, 2, 3, and 7. In some cases, genes encoding for three or more enzymes that catalyze consecutive reactions can be expressed within the microorganism. In some cases, genes encoding for four or more enzymes that catalyze consecutive reactions can be expressed within the microorganism. In some cases, genes encoding for five or more enzymes that catalyze consecutive reactions can be expressed within the microorganism. In some cases, genes encoding for six or more enzymes that catalyze consecutive reactions can be expressed within the microorganism. In some cases, genes encoding for seven or more enzymes that catalyze consecutive reactions can be expressed within the microorganism. In some cases, genes encoding for eight or more enzymes that catalyze consecutive reactions can be expressed within the microorganism. In some cases, genes encoding for nine or more enzymes that catalyze consecutive reactions can be expressed within the microorganism. In some cases, genes encoding for ten or more enzymes that catalyze consecutive reactions can be expressed within the microorganism.

### Isolated Nucleic Acids

The genes described herein can be in the form of an isolated polynucleic acid. In other words, the genes can be in forms that do not exist in nature, isolated from a chromosome. The isolated polynucleic acids can comprise a nucleic acid sequence of one or more of the following genes: i) α-ketoglutarate decarboxylase (kgd); ii) 4-hydroxybutyrate dehydrogenase (4hbD); iii) succinyl CoA synthease beta subunit (sucC); iv) succinyl CoA synthease alpha subunit (sucD); v) 4-hydroxybutyrate CoA transferase (cat2); (vi) aldehyde dehydrogenase (ald); (vii) alcohol dehydrogenase (adh); (viii) fumarate hydratase; and/or (ix) fumarate reductase. For example, the isolated polynucleic acid can comprise an α-ketoglutarate decarboxylase gene. The isolated polynucleic acid can comprise a 4-hydroxybutyrate dehydrogenase gene. The isolated polynucleic acid can comprise a succinyl CoA synthease beta subunit gene. The isolated polynucleic acid can comprise a succinyl CoA synthease alpha subunit gene. In some cases, the isolated polynucleic acid can comprise a 4-hydroxybutyrate CoA transferase gene. In other instances, the isolated polynucleic acid can comprise an aldehyde dehydrogenase gene. In some cases, the isolated polynucleic acid can comprise an alcohol dehydrogenase gene.

In some cases, the isolated polynucleic acid can encode for an α-ketoglutarate decarboxylase gene. For example, the isolated polynucleic acid can comprise a nucleotide sequence that is substantially similar to any one of SEQ ID NOs. 2, 4, 6, or 8. In some cases, the isolated polynucleic acid can comprise a nucleotide sequence that is at least 60% identical to any one of SEQ ID NOs. 2, 4, 6, or 8. In some cases, the isolated polynucleic acid can comprise a nucleotide sequence that is at least 65% identical to any one of SEQ ID NOs. 2, 4, 6, or 8. In some cases, the isolated polynucleic acid can comprise a nucleotide sequence that is at least 70% identical to any one of SEQ ID NOs. 2, 4, 6, or 8. In some cases, the isolated polynucleic acid can comprise a nucleotide sequence that is at least 75% identical to any one of SEQ ID NOs. 2, 4, 6, or 8. In some cases, the isolated polynucleic acid can comprise a nucleotide sequence that is at least 80% identical to any one of SEQ ID NOs. 2, 4, 6, or 8. In some cases, the isolated polynucleic acid can comprise a nucleotide sequence that is at least 81% identical to any one of SEQ ID NOs. 2, 4, 6, or 8. In some cases, the isolated polynucleic acid can comprise a nucleotide sequence that is at least 82% identical to any one of SEQ ID NOs. 2, 4, 6, or 8. In some cases, the isolated polynucleic acid can comprise a nucleotide sequence that is at least 83% identical to any one of SEQ ID NOs. 2, 4, 6, or 8. In some cases, the isolated polynucleic acid can comprise a nucleotide sequence that is at least 84% identical to any one of SEQ ID NOs. 2, 4, 6, or 8. In some cases, the isolated polynucleic acid can comprise a nucleotide sequence that is at least 85% identical to any one of SEQ ID NOs. 2, 4, 6, or 8. In some cases, the isolated polynucleic acid can comprise a nucleotide sequence that is at least 86% identical to any one of SEQ ID NOs. 2, 4, 6, or 8. In some cases, the isolated polynucleic acid can comprise a nucleotide sequence that is at least 87% identical to any one of SEQ ID NOs. 2, 4, 6, or 8. In some cases, the isolated polynucleic acid can comprise a nucleotide sequence that is at least 88% identical to any one of SEQ ID NOs. 2, 4, 6, or 8. In some cases, the isolated polynucleic acid can comprise a nucleotide sequence that is at least 89% identical to any one of SEQ ID NOs. 2, 4, 6, or 8. In some cases, the isolated polynucleic acid can comprise a nucleotide sequence that is at least 90% identical to any one of SEQ ID NOs. 2, 4, 6, or 8. In some cases, the isolated polynucleic acid can comprise a nucleotide sequence that is at least 91% identical to any one of SEQ ID NOs. 2, 4, 6, or 8. In some cases, the isolated polynucleic acid can comprise a nucleotide sequence that is at least 92% identical to any one of SEQ ID NOs. 2, 4, 6, or 8. In some cases, the isolated polynucleic acid can comprise a nucleotide sequence that is at least 93% identical to any one of SEQ ID NOs. 2, 4, 6, or 8. In some cases, the isolated polynucleic acid can comprise a nucleotide sequence that is at least 94% identical to any one of SEQ ID NOs. 2, 4, 6, or 8. In some cases, the isolated polynucleic acid can comprise a nucleotide sequence that is at least 95% identical to any one of SEQ ID NOs. 2, 4, 6, or 8. In some cases, the isolated polynucleic acid can comprise a nucleotide sequence that is at least 96% identical to any one of SEQ ID NOs. 2, 4, 6, or 8. In some cases, the isolated polynucleic acid can comprise a nucleotide sequence that is at least 97% identical to any one of SEQ ID NOs. 2, 4, 6, or 8. In some cases, the isolated polynucleic acid can comprise a nucleotide sequence that is at least 98% identical to any one of SEQ ID NOs. 2, 4, 6, or 8. In some cases, the isolated polynucleic acid can comprise a nucleotide sequence that is at least 99% identical to any one of SEQ ID NOs. 2, 4, 6, or 8. In some cases, the isolated polynucleic acid can comprise a nucleotide sequence that is any one of SEQ ID NOs. 2, 4, 6, or 8.

In some cases, the isolated polynucleic acid can encode for a 4-hydroxybutyrate dehydrogenase. For example, the isolated polynucleic acid can comprise a nucleotide sequence that is substantially similar to SEQ ID NO. 12 or 14. In some cases, the isolated polynucleic acid can comprise a nucleotide sequence that is at least 60% identical to SEQ ID NO. 12 or 14. In some cases, the isolated polynucleic acid can comprise a nucleotide sequence that is at least 65% identical to SEQ ID NO. 12 or 14. In some cases, the isolated polynucleic acid can comprise a nucleotide sequence that is at least 70% identical to SEQ ID NO. 12 or 14. In some cases, the isolated polynucleic acid can comprise a nucleotide sequence that is at least 75% identical to SEQ ID NO. 12 or 14. In some cases, the isolated polynucleic acid can comprise a nucleotide sequence that is at least 80% identical to SEQ ID NO. 12 or 14. In some cases, the isolated polynucleic acid can comprise a nucleotide sequence that is at least 81% identical to SEQ ID NO. 12 or 14. In some cases, the isolated polynucleic acid can comprise a nucleotide sequence that is at least 82% identical to SEQ ID NO. 12 or 14. In some cases, the isolated polynucleic acid can comprise a nucleotide sequence that is at least 83% identical to SEQ ID NO. 12 or 14. In some cases, the isolated polynucleic acid can comprise a nucleotide sequence that is at least 84% identical to SEQ ID NO. 12 or 14. In some cases, the isolated polynucleic acid can comprise a nucleotide sequence that is at least 85% identical to SEQ ID NO. 12 or 14. In some cases, the isolated polynucleic acid can comprise a nucleotide sequence that is at least 86% identical to SEQ ID NO. 12 or 14. In some cases, the isolated polynucleic acid can comprise a nucleotide sequence that is at least 87% identical to SEQ ID NO. 12 or 14. In some cases, the isolated polynucleic acid can comprise a nucleotide sequence that is at least 88% identical to SEQ ID NO. 12 or 14. In some cases, the isolated polynucleic acid can comprise a nucleotide sequence that is at least 89% identical to SEQ ID NO. 12 or 14. In some cases, the isolated polynucleic acid can comprise a nucleotide sequence that is at least 90% identical to SEQ ID NO. 12 or 14. In some cases, the isolated polynucleic acid can comprise a nucleotide sequence that is at least 91% identical to SEQ ID NO. 12 or 14. In some cases, the isolated polynucleic acid can comprise a nucleotide sequence that is at least 92% identical to SEQ ID NO. 12 or 14. In some cases, the isolated polynucleic acid can comprise a nucleotide sequence that is at least 93% identical to SEQ ID NO. 12 or 14. In some cases, the isolated polynucleic acid can comprise a nucleotide sequence that is at least 94% identical to SEQ ID NO. 12 or 14. In some cases, the isolated polynucleic acid can comprise a nucleotide sequence that is at least 95% identical to SEQ ID NO. 12 or 14. In some cases, the isolated polynucleic acid can comprise a nucleotide sequence that is at least 96% identical to SEQ ID NO. 12 or 14. In some cases, the isolated polynucleic acid can comprise a nucleotide sequence that is at least 97% identical to SEQ ID NO. 12 or 14. In some cases, the isolated polynucleic acid can comprise a nucleotide sequence that is at least 98% identical to SEQ ID NO. 12 or 14. In some cases, the isolated polynucleic acid can comprise a nucleotide sequence that is at least 99% identical to SEQ ID NO. 12 or 14. In some cases, the isolated polynucleic acid can comprise a nucleotide sequence that is identical to SEQ ID NO. 12 or 14.

In some cases, the isolated polynucleic acid can comprise a nucleotide sequence that encodes for a 4-hydroxybutyrate CoA transferase. In some cases, the isolated polynucleic acid can comprise a nucleotide sequence that is substantially similar to any one of SEQ ID NOs. 16, 18, or 20. For example, the isolated polynucleic acid can comprise a nucleotide sequence that is at least 60% identical to any one of SEQ ID NOs. 16, 18, or 20. In some cases, the isolated polynucleic acid can comprise a nucleotide sequence that is at least 65% identical to any one of SEQ ID NOs. 16, 18, or 20. In some cases, the isolated polynucleic acid can comprise a nucleotide sequence that is at least 70% identical to any one of SEQ ID NOs. 16, 18, or 20. In some cases, the isolated polynucleic acid can comprise a nucleotide sequence that is at least 75% identical to any one of SEQ ID NOs. 16, 18, or 20. In some cases, the isolated polynucleic acid can comprise a nucleotide sequence that is at least 80% identical to any one of SEQ ID NOs. 16, 18, or 20. In some cases, the isolated polynucleic acid can comprise a nucleotide sequence that is at least 81% identical to any one of SEQ ID NOs. 16, 18, or 20. In some cases, the isolated polynucleic acid can comprise a nucleotide sequence that is at least 82% identical to any one of SEQ ID NOs. 16, 18, or 20. In some cases, the isolated polynucleic acid can comprise a nucleotide sequence that is at least 83% identical to any one of SEQ ID NOs. 16, 18, or 20. In some cases, the isolated polynucleic acid can comprise a nucleotide sequence that is at least 84% identical to any one of SEQ ID NOs. 16, 18, or 20. In some cases, the isolated polynucleic acid can comprise a nucleotide sequence that is at least 85% identical to any one of SEQ ID NOs. 16, 18, or 20. In some cases, the isolated polynucleic acid can comprise a nucleotide sequence that is at least 86% identical to any one of SEQ ID NOs. 16, 18, or 20. In some cases, the isolated polynucleic acid can comprise a nucleotide sequence that is at least 87% identical to any one of SEQ ID NOs. 16, 18, or 20. In some cases, the isolated polynucleic acid can comprise a nucleotide sequence that is at least 88% identical to any one of SEQ ID NOs. 16, 18, or 20. In some cases, the isolated polynucleic acid can comprise a nucleotide sequence that is at least 89% identical to any one of SEQ ID NOs. 16, 18, or 20. In some cases, the isolated polynucleic acid can comprise a nucleotide sequence that is at least 90% identical to any one of SEQ ID NOs. 16, 18, or 20. In some cases, the isolated polynucleic acid can comprise a nucleotide sequence that is at least 91% identical to any one of SEQ ID NOs. 16, 18, or 20. In some cases, the isolated polynucleic acid can comprise a nucleotide sequence that is at least 92% identical to any one of SEQ ID NOs. 16, 18, or 20. In some cases, the isolated polynucleic acid can comprise a nucleotide sequence that is at least 93% identical to any one of SEQ ID NOs. 16, 18, or 20. In some cases, the isolated polynucleic acid can comprise a nucleotide sequence that is at least 94% identical to any one of SEQ ID NOs. 16, 18, or 20. In some cases, the isolated polynucleic acid can comprise a nucleotide sequence that is at least 95% identical to any one of SEQ ID NOs. 16, 18, or 20. In some cases, the isolated polynucleic acid can comprise a nucleotide sequence that is at least 96% identical to any one of SEQ ID NOs. 16, 18, or 20. In some cases, the isolated polynucleic acid can comprise a nucleotide sequence that is at least 97% identical to any one of SEQ ID NOs. 16, 18, or 20. In some cases, the isolated polynucleic acid can comprise a nucleotide sequence that is at least 98% identical to any one of SEQ ID NOs. 16, 18, or 20. In some cases, the isolated polynucleic acid can comprise a nucleotide sequence that is at least 99% identical to any one of SEQ ID NOs. 16, 18, or 20. In some cases, the isolated polynucleic acid can comprise a nucleotide sequence that is identical to any one of SEQ ID NOs. 16, 18, or 20.

In some cases, the isolated polynucleic acid can comprise a nucleotide sequence that encodes for an aldehyde dehydrogenase and/or alcohol dehydrogenase. In some cases, the isolated polynucleic acid can comprise a nucleotide sequence that is substantially similar to any one of SEQ ID NOs. 22, 24, 26, 28, 30, 32, 34, or 36. For example, the isolated polynucleic acid can comprise a nucleotide sequence that is at least 60% identical to any one of SEQ ID NOs. 22, 24, 26, 28, 30, 32, 34, or 36. In some cases, the isolated polynucleic acid can comprise a nucleotide sequence that is at least 65% identical to any one of SEQ ID NOs. 22, 24, 26, 28, 30, 32, 34, or 36. In some cases, the isolated polynucleic acid can comprise a nucleotide sequence that is at least 70% identical to any one of SEQ ID NOs. 22, 24, 26, 28, 30, 32, 34, or 36. In some cases, the isolated polynucleic acid can comprise a nucleotide sequence that is at least 75% identical to any one of SEQ ID NOs. 22, 24, 26, 28, 30, 32, 34, or 36. In some cases, the isolated polynucleic acid can comprise a nucleotide sequence that is at least 80% identical to any one of SEQ ID NOs. 22, 24, 26, 28, 30, 32, 34, or 36. In some cases, the isolated polynucleic acid can comprise a nucleotide sequence that is at least 81% identical to any one of SEQ ID NOs. 22, 24, 26, 28, 30, 32, 34, or 36. In some cases, the isolated polynucleic acid can comprise a nucleotide sequence that is at least 82% identical to any one of SEQ ID NOs. 22, 24, 26, 28, 30, 32, 34, or 36. In some cases, the isolated polynucleic acid can comprise a nucleotide sequence that is at least 83% identical to any one of SEQ ID NOs. 22, 24, 26, 28, 30, 32, 34, or 36. In some cases, the isolated polynucleic acid can comprise a nucleotide sequence that is at least 84% identical to any one of SEQ ID NOs. 22, 24, 26, 28, 30, 32, 34, or 36. In some cases, the isolated polynucleic acid can comprise a nucleotide sequence that is at least 85% identical to any one of SEQ ID NOs. 22, 24, 26, 28, 30, 32, 34, or 36. In some cases, the isolated polynucleic acid can comprise a nucleotide sequence that is at least 86% identical to any one of SEQ ID NOs. 22, 24, 26, 28, 30, 32, 34, or 36. In some cases, the isolated polynucleic acid can comprise a nucleotide sequence that is at least 87% identical to any one of SEQ ID NOs. 22, 24, 26, 28, 30, 32, 34, or 36. In some cases, the isolated polynucleic acid can comprise a nucleotide sequence that is at least 88% identical to any one of SEQ ID NOs. 22, 24, 26, 28, 30, 32, 34, or 36. In some cases, the isolated polynucleic acid can comprise a nucleotide sequence that is at least 89% identical to any one of SEQ ID NOs. 22, 24, 26, 28, 30, 32, 34, or 36. In some cases, the isolated polynucleic acid can comprise a nucleotide sequence that is at least 90% identical to any one of SEQ ID NOs. 22, 24, 26, 28, 30, 32, 34, or 36. In some cases, the isolated polynucleic acid can comprise a nucleotide sequence that is at least 91% identical to any one of SEQ ID NOs. 22, 24, 26, 28, 30, 32, 34, or 36. In some cases, the isolated polynucleic acid can comprise a nucleotide sequence that is at least 92% identical to any one of SEQ ID NOs. 22, 24, 26, 28, 30, 32, 34, or 36. In some cases, the isolated polynucleic acid can comprise a nucleotide sequence that is at least 93% identical to any one of SEQ ID NOs. 22, 24, 26, 28, 30, 32, 34, or 36. In some cases, the isolated polynucleic acid can comprise a nucleotide sequence that is at least 94% identical to any one of SEQ ID NOs. 22, 24, 26, 28, 30, 32, 34, or 36. In some cases, the isolated polynucleic acid can comprise a nucleotide sequence that is at least 95% identical to any one of SEQ ID NOs. 22, 24, 26, 28, 30, 32, 34, or 36. In some cases, the isolated polynucleic acid can comprise a nucleotide sequence that is at least 96% identical to any one of SEQ ID NOs. 22, 24, 26, 28, 30, 32, 34, or 36. In some cases, the isolated polynucleic acid can comprise a nucleotide sequence that is at least 97% identical to any one of SEQ ID NOs. 22, 24, 26, 28, 30, 32, 34, or 36. In some cases, the isolated polynucleic acid can comprise a nucleotide sequence that is at least 98% identical to any one of SEQ ID NOs. 22, 24, 26, 28, 30, 32, 34, or 36. In some cases, the isolated polynucleic acid can comprise a nucleotide sequence that is at least 99% identical to any one of SEQ ID NOs. 22, 24, 26, 28, 30, 32, 34, or 36. In some cases, the isolated polynucleic acid can comprise a nucleotide sequence that is identical to any one of SEQ ID NOs. 22, 24, 26, 28, 30, 32, 34, or 36.

In some cases, the isolated polynucleic acid can comprise a nucleotide sequence that encodes for a succinyl CoA synthease beta subunit. In some cases, the isolated polynucleic acid can comprise a nucleotide sequence that is substantially similar to SEQ ID NO. 38. For example, the isolated polynucleic acid can comprise a nucleotide sequence that is at least 60% identical to SEQ ID NO. 38. In some cases, the isolated polynucleic acid can comprise a nucleotide sequence that is at least 65% identical to SEQ ID NO. 38. In some cases, the isolated polynucleic acid can comprise a nucleotide sequence that is at least 70% identical to SEQ ID NO. 38. In some cases, the isolated polynucleic acid can comprise a nucleotide sequence that is at least 75% identical to SEQ ID NO. 38. In some cases, the isolated polynucleic acid can comprise a nucleotide sequence that is at least 80% identical to SEQ ID NO. 38. In some cases, the isolated polynucleic acid can comprise a nucleotide sequence that is at least 81% identical to SEQ ID NO. 38. In some cases, the isolated polynucleic acid can comprise a nucleotide sequence that is at least 82% identical to SEQ ID NO. 38. In some cases, the isolated polynucleic acid can comprise a nucleotide sequence that is at least 83% identical to SEQ ID NO. 38. In some cases, the isolated polynucleic acid can comprise a nucleotide sequence that is at least 84% identical to SEQ ID NO. 38. In some cases, the isolated polynucleic acid can comprise a nucleotide sequence that is at least 85% identical to SEQ ID NO. 38. In some cases, the isolated polynucleic acid can comprise a nucleotide sequence that is at least 86% identical to SEQ ID NO. 38. In some cases, the isolated polynucleic acid can comprise a nucleotide sequence that is at least 87% identical to SEQ ID NO. 38. In some cases, the isolated polynucleic acid can comprise a nucleotide sequence that is at least 88% identical to SEQ ID NO. 38. In some cases, the isolated polynucleic acid can comprise a nucleotide sequence that is at least 89% identical to SEQ ID NO. 38. In some cases, the isolated polynucleic acid can comprise a nucleotide sequence that is at least 90% identical to SEQ ID NO. 38. In some cases, the isolated polynucleic acid can comprise a nucleotide sequence that is at least 91% identical to SEQ ID NO. 38. In some cases, the isolated polynucleic acid can comprise a nucleotide sequence that is at least 92% identical to SEQ ID NO. 38. In some cases, the isolated polynucleic acid can comprise a nucleotide sequence that is at least 93% identical to SEQ ID NO. 38. In some cases, the isolated polynucleic acid can comprise a nucleotide sequence that is at least 94% identical to SEQ ID NO. 38. In some cases, the isolated polynucleic acid can comprise a nucleotide sequence that is at least 95% identical to SEQ ID NO. 38. In some cases, the isolated polynucleic acid can comprise a nucleotide sequence that is at least 96% identical to SEQ ID NO. 38. In some cases, the isolated polynucleic acid can comprise a nucleotide sequence that is at least 97% identical to SEQ ID NO. 38. In some cases, the isolated polynucleic acid can comprise a nucleotide sequence that is at least 98% identical to SEQ ID NO. 38. In some cases, the isolated polynucleic acid can comprise a nucleotide sequence that is at least 99% identical to SEQ ID NO. 38. In some cases, the isolated polynucleic acid can comprise a nucleotide sequence that is identical to SEQ ID NO. 38.

In some cases, the isolated polynucleic acid can comprise a nucleotide sequence that encodes for a succinyl CoA synthease alpha subunit. In some cases, the isolated polynucleic acid can comprise a nucleotide sequence that is substantially similar to any one of SEQ ID NOs. 40, 43, or 44. For example, the isolated polynucleic acid can comprise a nucleotide sequence that is at least 60% identical to any one of SEQ ID NOs. 40, 43, or 44. In some cases, the isolated polynucleic acid can comprise a nucleotide sequence that is at least 65% identical to any one of SEQ ID NOs. 40, 43, or 44. In some cases, the isolated polynucleic acid can comprise a nucleotide sequence that is at least 70% identical to any one of SEQ ID NOs. 40, 43, or 44. In some cases, the isolated polynucleic acid can comprise a nucleotide sequence that is at least 75% identical to any one of SEQ ID NOs. 40, 43, or 44. In some cases, the isolated polynucleic acid can comprise a nucleotide sequence that is at least 80% identical to any one of SEQ ID NOs. 40, 43, or 44. In some cases, the isolated polynucleic acid can comprise a nucleotide sequence that is at least 81% identical to any one of SEQ ID NOs. 40, 43, or 44. In some cases, the isolated polynucleic acid can comprise a nucleotide sequence that is at least 82% identical to any one of SEQ ID NOs. 40, 43, or 44. In some cases, the isolated polynucleic acid can comprise a nucleotide sequence that is at least 83% identical to any one of SEQ ID NOs. 40, 43, or 44. In some cases, the isolated polynucleic acid can comprise a nucleotide sequence that is at least 84% identical to any one of SEQ ID NOs. 40, 43, or 44. In some cases, the isolated polynucleic acid can comprise a nucleotide sequence that is at least 85% identical to any one of SEQ ID NOs. 40, 43, or 44. In some cases, the isolated polynucleic acid can comprise a nucleotide sequence that is at least 86% identical to any one of SEQ ID NOs. 40, 43, or 44. In some cases, the isolated polynucleic acid can comprise a nucleotide sequence that is at least 87% identical to any one of SEQ ID NOs. 40, 43, or 44. In some cases, the isolated polynucleic acid can comprise a nucleotide sequence that is at least 88% identical to any one of SEQ ID NOs. 40, 43, or 44. In some cases, the isolated polynucleic acid can comprise a nucleotide sequence that is at least 89% identical to any one of SEQ ID NOs. 40, 43, or 44. In some cases, the isolated polynucleic acid can comprise a nucleotide sequence that is at least 90% identical to any one of SEQ ID NOs. 40, 43, or 44. In some cases, the isolated polynucleic acid can comprise a nucleotide sequence that is at least 91% identical to any one of SEQ ID NOs. 40, 43, or 44. In some cases, the isolated polynucleic acid can comprise a nucleotide sequence that is at least 92% identical to any one of SEQ ID NOs. 40, 43, or 44. In some cases, the isolated polynucleic acid can comprise a nucleotide sequence that is at least 93% identical to any one of SEQ ID NOs. 40, 43, or 44. In some cases, the isolated polynucleic acid can comprise a nucleotide sequence that is at least 94% identical to any one of SEQ ID NOs. 40, 43, or 44. In some cases, the isolated polynucleic acid can comprise a nucleotide sequence that is at least 95% identical to any one of SEQ ID NOs. 40, 43, or 44. In some cases, the isolated polynucleic acid can comprise a nucleotide sequence that is at least 96% identical to any one of SEQ ID NOs. 40, 43, or 44. In some cases, the isolated polynucleic acid can comprise a nucleotide sequence that is at least 97% identical to any one of SEQ ID NOs. 40, 43, or 44. In some cases, the isolated polynucleic acid can comprise a nucleotide sequence that is at least 98% identical to any one of SEQ ID NOs. 40, 43, or 44. In some cases, the isolated polynucleic acid can comprise a nucleotide sequence that is at least 99% identical to any one of SEQ ID NOs. 40, 43, or 44. In some cases, the isolated polynucleic acid can comprise a nucleotide sequence that is identical to any one of SEQ ID NOs. 40, 43, or 44.

In some cases, the isolated polynucleic acid can comprise a nucleotide sequence that encodes for a fumarate hydratase. In some cases, the isolated polynucleic acid can comprise a nucleotide sequence that is substantially similar to any one of SEQ ID NOs. 46, 48, or 50. For example, the isolated polynucleic acid can comprise a nucleotide sequence that is at least 60% identical to any one of SEQ ID NOs. 46, 48, or 50. In some cases, the isolated polynucleic acid can comprise a nucleotide sequence that is at least 65% identical to any one of SEQ ID NOs. 46, 48, or 50. In some cases, the isolated polynucleic acid can comprise a nucleotide sequence that is at least 70% identical to any one of SEQ ID NOs. 46, 48, or 50. For example, the isolated polynucleic acid can comprise a nucleotide sequence that is at least 75% identical to any one of SEQ ID NOs. 46, 48, or 50. In some cases, the isolated polynucleic acid can comprise a nucleotide sequence that is at least 80% identical to any one of SEQ ID NOs. 46, 48, or 50. For example, the isolated polynucleic acid can comprise a nucleotide sequence that is at least 85% identical to any one of SEQ ID NOs. 46, 48, or 50. In some cases, the isolated polynucleic acid can comprise a nucleotide sequence that is at least 90% identical to any one of SEQ ID NOs. 46, 48, or 50. In some cases, the isolated polynucleic acid can comprise a nucleotide sequence that is at least 91% identical to any one of SEQ ID NOs. 46, 48, or 50. In some cases, the isolated polynucleic acid can comprise a nucleotide sequence that is at least 92% identical to any one of SEQ ID NOs. 46, 48, or 50. In some cases, the isolated polynucleic acid can comprise a nucleotide sequence that is at least 93% identical to any one of SEQ ID NOs. 46, 48, or 50. In some cases, the isolated polynucleic acid can comprise a nucleotide sequence that is at least 94% identical to any one of SEQ ID NOs. 46, 48, or 50. In some cases, the isolated polynucleic acid can comprise a nucleotide sequence that is at least 95% identical to any one of SEQ ID NOs. 46, 48, or 50. In some cases, the isolated polynucleic acid can comprise a nucleotide sequence that is at least 96% identical to any one of SEQ ID NOs. 46, 48, or 50. In some cases, the isolated polynucleic acid can comprise a nucleotide sequence that is at least 97% identical to any one of SEQ ID NOs. 46, 48, or 50. In some cases, the isolated polynucleic acid can comprise a nucleotide sequence that is at least 98% identical to any one of SEQ ID NOs. 46, 48, or 50. In some cases, the isolated polynucleic acid can comprise a nucleotide sequence that is at least 99% identical to any one of SEQ ID NOs. 46, 48, or 50. In some cases, the isolated polynucleic acid can comprise a nucleotide sequence that is any one of SEQ ID NOs. 46, 48, or 50.

In some cases, the isolated polynucleic acid can comprise a nucleotide sequence that encodes for a fumarate reductase. In some cases, the isolated polynucleic acid can comprise a nucleotide sequence that is substantially similar to any one of SEQ ID NOs. 52, 54, 56, or 58. For example, the isolated polynucleic acid can comprise a nucleotide sequence that is at least 60% identical to any one of SEQ ID NOs. 52, 54, 56, or 58. For example, the isolated polynucleic acid can comprise a nucleotide sequence that is at least 65% identical to any one of SEQ ID NOs. 52, 54, 56, or 58. In some cases, the isolated polynucleic acid can comprise a nucleotide sequence that is at least 70% identical to any one of SEQ ID NOs. 52, 54, 56, or 58. For example, the isolated polynucleic acid can comprise a nucleotide sequence that is at least 75% identical to any one of SEQ ID NOs. 52, 54, 56, or 58. In some cases, the isolated polynucleic acid can comprise a nucleotide sequence that is at least 80% identical to any one of SEQ ID NOs. 52, 54, 56, or 58. For example, the isolated polynucleic acid can comprise a nucleotide sequence that is at least 85% identical to any one of SEQ ID NOs. 52, 54, 56, or 58. In some cases, the isolated polynucleic acid can comprise a nucleotide sequence that is at least 90% identical to any one of SEQ ID NOs. 52, 54, 56, or 58. In some cases, the isolated polynucleic acid can comprise a nucleotide sequence that is at least 91% identical to any one of SEQ ID NOs. 52, 54, 56, or 58. In some cases, the isolated polynucleic acid can comprise a nucleotide sequence that is at least 92% identical to any one of SEQ ID NOs. 52, 54, 56, or 58. In some cases, the isolated polynucleic acid can comprise a nucleotide sequence that is at least 93% identical to any one of SEQ ID NOs. 52, 54, 56, or 58. In some cases, the isolated polynucleic acid can comprise a nucleotide sequence that is at least 94% identical to any one of SEQ ID NOs. 52, 54, 56, or 58. In some cases, the isolated polynucleic acid can comprise a nucleotide sequence that is at least 95% identical to any one of SEQ ID NOs. 52, 54, 56, or 58. In some cases, the isolated polynucleic acid can comprise a nucleotide sequence that is at least 96% identical to any one of SEQ ID NOs. 52, 54, 56, or 58. In some cases, the isolated polynucleic acid can comprise a nucleotide sequence that is at least 97% identical to any one of SEQ ID NOs. 52, 54, 56, or 58. In some cases, the isolated polynucleic acid can comprise a nucleotide sequence that is at least 98% identical to any one of SEQ ID NOs. 52, 54, 56, or 58. In some cases, the isolated polynucleic acid can comprise a nucleotide sequence that is at least 99% identical to any one of SEQ ID NOs. 52, 54, 56, or 58. In some cases, In some cases, the isolated polynucleic acid can comprise a nucleotide sequence that encodes for a fumarate hydratase. In some cases, the isolated polynucleic acid can comprise a nucleotide sequence that is any one of SEQ ID NOs. 52, 54, 56, or 58.

### II. Method of Making the Genetically Modified Microorganisms

The genetically modified microorganisms above can be made by a variety of ways. A microorganism may be modified (e.g., genetically engineered) by any method to comprise and/or express one or more polynucleotides encoding for enzymes in a pathway that catalyze a conversion of a fermentable carbon source (e.g., a C1 carbon) to one or more intermediates in a pathway for the production of a multicarbon product such as 1,4-BDO. Such enzymes may include any or all of those enzymes as set forth in FIG. 1, 2, 3, or 7. For example, one or more of any of the genes above can be inserted into a microorganism. The genes can be inserted by an expression vector. The one or more genes can also be stably integrated into the genome of the microorganism.

The microorganism used in this method can be any described above, including but not limited to a prokaryote. Other microorganisms such as bacteria, yeast, or algae can be used. One microorganism of particular interest is a methanotroph, such as a methanotroph from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, or Methylacidiphilum. One desired species can include a Methylococcus capsulatus.

An exemplary method of making a genetically modified microorganism disclosed herein is contacting (or transforming) a microorganism with a nucleic acid that expresses at least one heterologous genes from: GROUP 1, GROUP 2, and/or GROUP 3. For example, the genes can include: i) α-ketoglutarate decarboxylase (kgd); ii) 4-hydroxybutyrate dehydrogenase (4hbD); iii) 4-hydroxybutyrate CoA transferase (cat2); (iv) aldehyde dehydrogenase (ald); (v) alcohol dehydrogenase (adh); (vi) succinyl CoA synthease beta subunit (sucC); (vii) succinyl CoA synthease alpha subunit (sucD); or (viii) any combination thereof. The genes can also include a fumarate hydratase or a fumarate reductase. The microorganism can be any microorganism that is capable of converting a C1 carbon to a multicarbon product. In some cases, the multicarbon product is 1,4-BDO. The microorganism can be any one of the microorganism described throughout the patent application.

The one or more genes that are inserted into a microorganism can be heterologous to the microorganism itself. For example, if the microorganism is a methanotroph, the one or more genes that are inserted can be from yeast, a bacterium, or a different species of methanotroph. Further, the one or more genes can be endogenously part of the genome of the microorganism.

### Techniques for Genetic Modification

The microorganisms disclosed herein may be genetically engineered by using classic microbiological techniques. Some of such techniques are generally disclosed, for example, in Sambrook et al., 1989, Molecular Cloning: A Laboratory Manual, Cold Spring Harbor Labs Press.

The genetically modified microorganisms disclosed herein may include a polynucleotide that has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of a gene to increase gene expression can include maintaining the gene(s) on replicating plasmids or integrating the cloned gene(s) into the genome of the production microorganism. Furthermore, increasing the expression of desired genes can include operatively linking the cloned gene(s) to native or heterologous transcriptional control elements.

Where desired, the expression of one or more of the enzymes provided herein is under the control of a regulatory sequence that controls directly or indirectly the enzyme expression in a time-dependent fashion during the fermentation. Inducible promoters can be used to achieve this.

In some cases, a microorganism is transformed or transfected with a genetic vehicle, such as an expression vector comprising a heterologous polynucleotide sequence coding for the enzymes are provided herein.

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

### Transfection

Standard transfection techniques can be used to insert genes into a microorganism. As used herein, the term “transfection” or “transformation” can refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into microorganisms. Examples of transfection techniques include, but are not limited to, calcium phosphate precipitation, DEAE-dextran-mediated transfection, lipofection, electroporation, microinjection, rubidium chloride or polycation mediated transfection, protoplast fusion, and sonication. The transfection method that provides optimal transfection frequency and expression of the construct in the particular host cell line and type is favored. For stable transfectants, the constructs are integrated so as to be stably maintained within the host chromosome. In some cases, the preferred transfection is a stable transfection.

### Transformation

Expression vectors or other nucleic acids may be introduced to selected microorganisms by any of a number of suitable methods. For example, vector constructs may be introduced to appropriate cells by any of a number of transformation methods for plasmid vectors. Standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to certain types of cells, the method used may depend upon the form of the vector. Plasmid vectors may be introduced by any of a of transfection methods, including, for example, lipid-number mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. Many companies offer kits and ways for this type of transfection.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation.

Microorganisms can be transformed or transfected with the above-described expression or vectors for production of one or more enzymes as disclosed herein or with polynucleotides coding for one or more enzymes as disclosed herein and cultured in nutrient media modified as appropriate for the specific microorganism, inducing promoters, selecting transformants, or amplifying the genes encoding the desired sequences.

For example, within the context of a methanotroph, electroporation methods can be used to deliver an expression vector.

Expression of a vector (and the gene contained in the vector) can be verified by an expression assay, for example, qPCR or by measuring levels of RNA. Expression level can be indicative also of copy number. For example, if expression levels are extremely high, this can indicate that more than one copy of a gene was integrated in a genome. Alternatively, high expression can indicate that a gene was integrated in a highly transcribed area, for example, near a highly expressed promoter. Expression can also be verified by measuring protein levels, such as through Western blotting.

### CRISPR/cas System

Methods that require any of the genes described herein can take advantage of pinpoint insertion of genes or the deletion of genes (or parts of genes). Methods described herein can take advantage of a CRISPR/cas system. For example, double-strand breaks (DSBs) can be generated using a CRISPR/cas system, e.g., a type II CRISPR/cas system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

A vector can be operably linked to an enzyme-coding sequence encoding a CRISPR enzyme, such as a Cas protein. Cas proteins that can be used include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence. A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used.

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the ammo-terminus, about or more than about 1, 2, 3, 4, 5, 6, 7, 8, 9, 10 NLSs at or near the carboxy-terminus, or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise at most 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Guide RNA

As used herein, the term “guide RNA” and its grammatical equivalents can refer to an RNA which can be specific for a target DNA and can form a complex with Cas protein. An RNA/Cas complex can assist in “guiding” Cas protein to a target DNA.

A method disclosed herein also can comprise introducing into a cell or embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-chain RNA, or single guide RNA (sgRNA) formed by fusion of a portion (e.g., a functional portion) of crRNA and tracrRNA. A guide RNA can also be a dualRNA comprising a crRNA and a tracrRNA. Furthermore, a crRNA can hybridize with a target DNA.

As discussed above, a guide RNA can be an expression product. For example, a DNA that encodes a guide RNA can be a vector comprising a sequence coding for the guide RNA. A guide RNA can be transferred into a cell or microorganism by transfecting the cell or microorganism with an isolated guide RNA or plasmid DNA comprising a sequence coding for the guide RNA and a promoter. A guide RNA can also be transferred into a cell or microorganism in other way, such as using virus-mediated gene delivery.

A guide RNA can be isolated. For example, a guide RNA can be transfected in the form of an isolated RNA into a cell or microorganism. A guide RNA can be prepared by in vitro transcription using any in vitro transcription system known in the art. A guide RNA can be transferred to a cell in the form of isolated RNA rather than in the form of plasmid comprising encoding sequence for a guide RNA.

A guide RNA can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA can be complementary to sequence at a target site in a chromosomal sequence such that the first region of the guide RNA can base pair with the target site. In some cases, a first region of a guide RNA can comprise from or from about 10 nucleotides to 25 nucleotides (i.e., from 10 nts to 25 nts; or from about 10 nts to about 25 nts; or from 10 nts to about 25 nts; or from about 10 nts to 25 nts) or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more nucleotides in length. Sometimes, a first region of a guide RNA can be or can be about 19, 20, or 21 nucleotides in length.

A guide RNA can also comprises a second region that forms a secondary structure. For example, a secondary structure formed by a guide RNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A guide RNA can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than or more than about 4 nucleotides in length. For example, the length of a third region can range from or from about 5 to 60 nucleotides in length.

A guide RNA can be introduced into a cell or embryo as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). Plasmid vectors that can be used to express guide RNA include, but are not limited to, px330 vectors and px333 vectors. In some cases, a plasmid vector (e.g., px333 vector) can comprise two guide RNA-encoding DNA sequences.

A DNA sequence encoding a guide RNA can also be part of a vector. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. A DNA molecule encoding a guide RNA can also be linear. A DNA molecule encoding a guide RNA can also be circular.

When DNA sequences encoding an RNA-guided endonuclease and a guide RNA arc introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

### Site Specific Insertion

Inserting one or more genes in any of the methods disclosed herein can be site-specific. For example, one or more genes can be inserted adjacent to a promoter.

Modification of a targeted locus of a microorganism can be produced by introducing DNA into microorganisms, where the DNA has homology to the target locus. DNA can include a marker gene, allowing for selection of cells comprising the integrated construct. Homologous DNA in a target vector can recombine with DNA at a target locus. A marker gene can be flanked on both sides by homologous DNA sequences, a 3′ recombination arm, and a 5′ recombination arm.

A variety of enzymes can catalyze insertion of foreign DNA into a microorganism genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, Φ31 integrase (a serine recombinase derived from Streptomyces phage Φ31), or bacteriophage derived site-specific recombinases (including Flp, lambda integrase, bacteriophage HK022 recombinase, bacteriophage R4 integrase and phage TP901-1 integrase).

The CRISPR/Cas system can be used to perform site specific insertion. For example, a nick on an insertion site in the genome can be made by CRISPR/cas to facilitate the insertion of a transgene at the insertion site.

The methods described herein, can utilize techniques which can be used to allow a DNA or RNA construct entry into a host cell include, but are not limited to, calcium phosphate/DNA co-precipitation, microinjection of DNA into a nucleus, electroporation, bacterial protoplast fusion with intact cells, transfection, lipofection, infection, particle bombardment, sperm mediated gene transfer, or any other technique.

Certain aspects disclosed herein can utilize vectors (including the ones described above). Any plasmids and vectors can be used as long as they are replicable and viable in a selected host microorganism. Vectors known in the art and those commercially available (and variants or derivatives thereof) can be engineered to include one or more recombination sites for use in the methods. Vectors that can be used include, but not limited to eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZcoSV, pcDNA3, pREP4, pCEP4, and pEBVHis (Invitrogen, Corp.), and variants or derivatives thereof.

These vectors can be used to express a gene or portion of a gene of interest. A gene of portion or a gene can be inserted by using known methods, such as restriction enzyme-based techniques.

### III. Other Methods

### Making Useful Chemicals

The genetically modified microorganisms described herein can be used to make chemicals and other products that are useful, including but not limited to α-ketoglutarate, succinate, citrate and isocitrate, γ-hydroxybutyrate, 4-hydroxybutyraldehyde, 1,4-BDO, tetrahydrofuran (THF), polybutylene terephthalate (PBT), and polyurethanes.

The microorganism can be any of the microorganisms discussed throughout including but not limited to a prokaryote, such as a methanotroph.

The carbon substrate can be any carbon substrate discussed throughout including but not limited to a C1 carbon substrate, such as methane.

α-ketoglutarate

With regards to α-ketoglutarate, one method that is disclosed herein is a method of making α-ketoglutarate comprising: (a) contacting a genetically modified microorganism with a carbon substrate (e.g., a C1 carbon substrate), where the microorganism comprises at least one heterologous gene encoding for an isocitrate dehydrogenase (icdA); and (b) growing the microorganism to produce α-ketoglutarate. The microorganism can be a microorganism that is capable of converted a C1 carbon into a multicarbon product. For example, a methanotroph can be used. Further any of the genetically modified microorganisms described throughout can be used. The α-ketoglutarate produced can be substantially pure. The α-ketoglutarate that is produced can be recovered. Additionally, non-α-ketoglutarate products (i.e., by-products) can also be recovered.

The α-ketoglutarate can be further processed by the same microorganism, a different microorganism, or outside a microorganism (i.e., in vitro) through an α-ketoglutarate decarboxylase (kgd) and/or α-ketoglutarate dehydrogenase. The same microorganism can comprise an α-ketoglutarate decarboxylase (kgd) and/or α-ketoglutarate dehydrogenase. In other instances, a different microorganism can comprise an α-ketoglutarate decarboxylase (kgd) and/or α-ketoglutarate dehydrogenase or the α-ketoglutarate decarboxylase (kgd) and/or α-ketoglutarate dehydrogenase are isolated from a cell. If the α-ketoglutarate decarboxylase (kgd) and/or α-ketoglutarate dehydrogenase is in a different microorganism or is isolated from a cell, the microorganism/isolated enzyme can convert α-ketoglutarate that is in the culture media (either by supplemental addition or by secretion by a α-ketoglutarate producing microorganism). The conversion of α-ketoglutarate by an α-ketoglutarate decarboxylase (kgd) can produce succinate semialdehyde. The conversion of α-ketoglutarate by an α-ketoglutarate dehydrogenase can produce succinate CoA.

Further conversion of succinate semialdehyde or succinate CoA into various products, such as γ-hydroxybutyrate or succinate semialdehyde can occur through different fermentation processes or by different catalytic conversions.

### Succinate

With regards to succinate, one method that is disclosed herein is a method of making succinate comprising: (a) contacting a genetically modified microorganism with a carbon substrate (e.g., a C1 carbon substrate), where the microorganism comprises a heterologous gene encoding for succinyl-CoA synthetase; and (b) growing the microorganism to produce succinate. Another method that is disclosed herein is a method of making succinate comprising: (a) contacting a genetically modified microorganism with a carbon substrate (e.g., a C1 carbon substrate), where the microorganism comprises a heterologous gene encoding for fumarate reductase; and (b) growing the microorganism to produce succinate. The microorganism can be a microorganism that is capable of converting a C1 carbon into a multicarbon product. For example, a methanotroph can be used. Further any of the genetically modified microorganisms described throughout can be used. The succinate produced can be substantially pure. The succinate that is produced can be recovered. Additionally, non-succinate products (i.e., by-products) can also be recovered.

The succinate can be further processed by the same microorganism, a different microorganism, or outside a microorganism (i.e., in vitro) through a succinyl CoA synthease beta subunit (SucC). The same microorganism can comprise a succinyl CoA synthease beta subunit. In other instances, a different microorganism can comprise a succinyl CoA synthease beta subunit or the succinyl CoA synthease beta subunit is isolated from a cell. If the succinyl CoA synthease beta subunit is in a different microorganism or is isolated from a cell, the microorganism/isolated enzyme can convert succinate that is in the culture media (either by supplemental addition or by secretion by a succinate producing microorganism). The conversion of succinate by a succinyl CoA synthease beta subunit can produce succinyl CoA.

Further conversion of succinyl CoA into various products can occur through different fermentation processes or by different catalytic conversions.

### Fumarate

With regards to fumarate, one method that is disclosed herein is a method of making fumarate comprising: (a) contacting a genetically modified microorganism with a carbon substrate (e.g., a C1 carbon substrate), where the microorganism comprises a heterologous gene encoding for fumarate hydratase; and (b) growing the microorganism to produce fumarate. The microorganism can be a microorganism that is capable of converting a C1 carbon into a multicarbon product. For example, a methanotroph can be used. Further any of the genetically modified microorganisms described throughout can be used. The fumarate produced can be substantially pure. The fumarate that is produced can be recovered. Additionally, non-fumarate products (i.e., by-products) can also be recovered.

The fumarate can be further processed by the same microorganism, a different microorganism, or outside a microorganism (i.e., in vitro) through a fumarate reductase. The same microorganism can comprise a fumarate reductase. In other instances, a different microorganism can comprise a fumarate reductase or the fumarate reductase is isolated from a cell. If the fumarate reductase is in a different microorganism or is isolated from a cell, the microorganism/isolated enzyme can convert fumarate that is in the culture media (either by supplemental addition or by secretion by a fumarate producing microorganism). The conversion of fumarate by a fumarate reductase can produce succinate.

Further conversion of succinate into various products can occur through different fermentation processes or by different catalytic conversions.

### Citrate

With regards to citrate, one method that is disclosed herein is a method of making citrate comprising: (a) contacting a genetically modified microorganism with a carbon substrate (e.g., a C1 carbon substrate), where the microorganism comprises a heterologous gene encoding for citrate synthase; and (b) growing the microorganism to produce citrate. The microorganism can be a microorganism that is capable of converting a C1 carbon into a multicarbon product. For example, a methanotroph can be used. Further any of the genetically modified microorganisms described throughout can be used. The citrate produced can be substantially pure. The citrate that is produced can be recovered. Additionally, non-citrate products (i.e., by-products) can also be recovered.

The citrate can be further processed by the same microorganism, a different microorganism, or outside a microorganism (i.e., in vitro) through an aconitate hydratase 1. The same microorganism can comprise an aconitate hydratase 1. In other instances, a different microorganism can comprise an aconitate hydratase 1 or the aconitate hydratase 1 is isolated from a cell. If the an aconitate hydratase 1 is in a different microorganism or is isolated from a cell, the microorganism/isolated enzyme can convert citrate that is in the culture media (either by supplemental addition or by secretion by a citrate producing microorganism). The conversion of citrate by an aconitate hydratase 1 can produce isocitrate.

Further conversion of isocitrate into various products can occur through different fermentation processes or by different catalytic conversions.

### Isocitrate

With regards to isocitrate, one method that is disclosed herein is a method of making isocitrate comprising: (a) contacting a genetically modified microorganism with a carbon substrate (e.g., a C1 carbon substrate), where the microorganism comprises a heterologous gene encoding for aconitate hydratase 1; and (b) growing the microorganism to produce isocitrate. The microorganism can be a microorganism that is capable of converting a C1 carbon into a multicarbon product. For example, a methanotroph can be used. Further any of the genetically modified microorganisms described throughout can be used. The isocitrate produced can be substantially pure. The isocitrate that is produced can be recovered. Additionally, non-isocitrate products (i.e., by-products) can also be recovered.

The isocitrate can be further processed by the same microorganism, a different microorganism, or outside a microorganism (i.e., in vitro) through an isocitrate dehydrogenase. The same microorganism can comprise an isocitrate dehydrogenase. In other instances, a different microorganism can comprise an isocitrate dehydrogenase or the isocitrate dehydrogenase is isolated from a cell. If the isocitrate dehydrogenase is in a different microorganism or is isolated from a cell, the microorganism/isolated enzyme can convert isocitrate that is in the culture media (either by supplemental addition or by secretion by an isocitrate producing microorganism). The conversion of isocitrate by an isocitrate dehydrogenase can produce α-ketoglutarate.

Further conversion of α-ketoglutarate into various products can occur through different fermentation processes or by different catalytic conversions.

γ-hydroxybutyrate

With regards to γ-hydroxybutyrate, one method that is disclosed herein is a method of making γ-hydroxybutyrate comprising: (a) contacting a genetically modified microorganism with a carbon substrate (e.g., a C1 carbon substrate), where the microorganism comprises a heterologous gene encoding for 4-hydroxybutyrate dehydrogenase (4hbD); and (b) growing the microorganism to produce γ-hydroxybutyrate. The microorganism can be a microorganism that is capable of converting a C1 carbon into a multicarbon product. For example, a methanotroph can be used. Further any of the genetically modified microorganisms described throughout can be used. The γ-hydroxybutyrate produced can be substantially pure. The γ-hydroxybutyrate that is produced can be recovered. Additionally, non-γ-hydroxybutyrate products (i.e., by-products) can also be recovered.

The γ-hydroxybutyrate can be further processed by the same microorganism, a different microorganism, or outside a microorganism (i.e., in vitro) through a 4-hydroxybutyrate CoA transferase (Cat2). The same microorganism can comprise a 4-hydroxybutyrate CoA transferase. In other instances, a different microorganism can comprise a 4-hydroxybutyrate CoA transferase or the 4-hydroxybutyrate CoA transferase is isolated from a cell. If the 4-hydroxybutyrate CoA transferase is in a different microorganism or is isolated from a cell, the microorganism/isolated enzyme can convert γ-hydroxybutyrate that is in the culture media (either by supplemental addition or by secretion by a γ-hydroxybutyrate producing microorganism). The conversion of γ-hydroxybutyrate by 4-hydroxybutyrate CoA transferase can produce 4-hydroxybutyryl CoA.

Further conversion of 4-hydroxybutyryl CoA into various products can occur through different fermentation processes or by different catalytic conversions.

4-hydroxybutyraldehyde

With regards to 4-hydroxybutyraldehyde, one method that is disclosed herein is a method of making 4-hydroxybutyraldehyde comprising: (a) contacting a genetically modified microorganism with a carbon substrate (e.g., a C1 carbon substrate), where the microorganism comprises a heterologous gene encoding for an aldehyde dehydrogenase; and (b) growing the microorganism to produce 4-hydroxybutyraldehyde. The microorganism can be a microorganism that is capable of converting a C1 carbon into a multicarbon product. For example, a methanotroph can be used. Further any of the genetically modified microorganisms described throughout can be used. The 4-hydroxybutyraldehyde produced can be substantially pure. The 4-hydroxybutyraldehyde that is produced can be recovered. Additionally, non-4-hydroxybutyraldehyde products (i.e., by-products) can also be recovered.

The 4-hydroxybutyraldehyde can be further processed by the same microorganism, a different microorganism, or outside a microorganism (i.e., in vitro) through an alcohol dehydrogenase. The same microorganism can comprise an alcohol dehydrogenase. In other instances, a different microorganism can comprise an alcohol dehydrogenase or the alcohol dehydrogenase is isolated from a cell. If the alcohol dehydrogenase is in a different microorganism or is isolated from a cell, the microorganism/isolated enzyme can convert 4-hydroxybutyraldehyde that is in the culture media (either by supplemental addition or by secretion by a 4-hydroxybutyraldehyde producing microorganism). The conversion of 4-hydroxybutyraldehyde by an alcohol dehydrogenase can produce 4-hydroxybutyryl CoA.

Further conversion of 4-hydroxybutyryl CoA into various products can occur through different fermentation processes or by different catalytic conversions.

### 1,4-BDO

With regards to 1,4-BDO, one method disclosed herein is a method of making 1,4-BDO comprising (a) contacting a genetically modified microorganism with a carbon substrate (e.g., a C1 carbon substrate), where the microorganism comprises at least one heterologous gene encoding for: (i) pyruvate dehydrogenase (aceEF); (ii) dihydrolipoyl dehydrogenase (lpdA); (iii) citrate synthase (gltA); (iv) aconitate hydratase 1 (acnA); (v) isocitrate dehydrogenase (icdA); (vi) kgd; (vii) 4-hydroxybutyrate dehydrogenase (4hbD); (viii) 4-hydroxybutyrate CoA transferase (cat2); (ix) aldehyde dehydrogenase (ald); (x) alcohol dehydrogenase (adh); (xi) succinyl CoA synthease beta subunit (sucC); (xii) succinyl CoA synthease alpha subunit (sucD); (xiii) fumarate hydratase (fum); (xiv) fumarate reductase (frd); or (xv) any combination thereof; and (b) growing the microorganism to produce 1,4-BDO.

The microorganism can be a microorganism that is capable of converting a C1 carbon into a multicarbon product. For example, a methanotroph can be used. Further any of the genetically modified microorganisms described throughout can be used.

The 1,4-BDO that is produced from this method can be substantially pure. The 1,4-BDO produced can be recovered. Additionally, non-1,4-BDO products (i.e., by-products) can also be recovered.

### Tetrahydrofuran (THE)

THF can be prepared by using a catalyst. For example, another method for making THF is a method comprising (a) contacting a genetically modified microorganism with a carbon substrate (e.g., a C1 carbon substrate), wherein the microorganism comprises at least one heterologous gene encoding for: (i) pyruvate dehydrogenase (accEF); (ii) dihydrolipoyl dehydrogenase (lpdA); (iii) citrate synthase (gltA); (iv) aconitate hydratase 1 (acnA); (v) isocitrate dehydrogenase (icdA); (vi) kgd; (vii) 4-hydroxybutyrate dehydrogenase (4hbD); (viii) 4-hydroxybutyrate CoA transferase (Cat2); (ix) aldehyde dehydrogenase (Ald); (x) alcohol dehydrogenase (Adh); (xi) succinyl CoA synthease beta subunit (SucC); (xii) succinyl CoA synthease alpha subunit (SucD); (xiii) fumarate hydratase (fum); (xiv) fumarate reductase (frd); or (xv) any combination thereof; and (b) growing the microorganism to produce 1,4-BDO, (c) contacting the 1,4-BDO from (b) with a catalyst to produce THF. In some instances, the 1,4-BDO can be isolated or purified before proceeding to (c).

The microorganism can be a microorganism that is capable of converting a C1 carbon into a multicarbon product. For example, a methanotroph can be used. Further any of the genetically modified microorganisms described throughout can be used.

The catalyst can be an enzymatic catalyst or a non-enzymatic catalyst. The catalyst can include any catalyst that is capable of producing THF. For example, THF can be obtained by the dehydration of 1,4-BDO in the presence of an acid catalyst such as sulfuric acid (see e.g., U.S. Pat. No. 7,465,816). Other catalysts (such as an alumina catalyst, a silica-alumina catalyst, an alumina-supported tungsten oxide catalyst, a heteropolyacid catalyst, or an zirconium sulfate catalyst) have been used to dehydrate 1,4-BDO to form THF.

Once converted, the THF can be isolated and/or purified. The THF can be substantially pure.

### Polybutylene Terephthalate (PBT)

PBT can be prepared by using a catalyst. For example, another method for making PBT is a method comprising (a) contacting a genetically modified microorganism with a carbon substrate (e.g., a C1 carbon substrate), wherein the microorganism comprises at least one heterologous gene encoding for: (i) pyruvate dehydrogenase (accEF); (ii) dihydrolipoyl dehydrogenase (lpdA); (iii) citrate synthase (gltA); (iv) aconitate hydratase 1 (acnA); (v) isocitrate dehydrogenase (icdA); (vi) kgd; (vii) 4-hydroxybutyrate dehydrogenase (4hbD); (viii) 4-hydroxybutyrate CoA transferase (Cat2); (ix) aldehyde dehydrogenase (Ald); (x) alcohol dehydrogenase (Adh); (xi) succinyl CoA synthease beta subunit (SucC); (xii) succinyl CoA synthease alpha subunit (SucD); (xiii) fumarate hydratase (fum); (xiv) fumarate reductase (frd); or (xv) any combination thereof; and (b) growing the microorganism to produce 1,4-BDO, (c) transesterfying the 1,4-BDO from (b) to produce PBT. The transesterification can be done using a dimethyl terephthalate (DMT). In some cases, the method can further comprise (d) polycondensation of the transesterified product when 1,4-BDO is in contact with DMT (e.g., the product of (c)).

The microorganism can be a microorganism that is capable of converting a C1 carbon into a multicarbon product. For example, a methanotroph can be used. Further any of the genetically modified microorganisms described throughout can be used.

Other methods can include a continuous production process of PBT wherein after transesterification of 1,4-BDO with DMT, a pre-polymerization process is implemented. This is followed by polycondensation.

In some instances, the 1,4-BDO can be isolated or purified before proceeding to the transesterification processes.

Once made, the PBT can be isolated and/or purified. The PBT can be substantially pure.

### Polyurethanes

Polyurethanes can be prepared by condensing 1,4-BDO with dicarboxylic acid/anhydride. For example, another method for making polyurethanes is a method comprising (a) contacting a genetically modified microorganism with a carbon substrate (e.g., a C1 carbon substrate), wherein the microorganism comprises at least one heterologous gene encoding for: (i) pyruvate dehydrogenase (accEF); (ii) dihydrolipoyl dehydrogenase (lpdA); (iii) citrate synthase (gltA); (iv) aconitate hydratase 1 (acnA); (v) isocitrate dehydrogenase (icdA); (vi) kgd; (vii) 4-hydroxybutyrate dehydrogenase (4hbD); (viii) 4-hydroxybutyrate CoA transferase (Cat2); (ix) aldehyde dehydrogenase (Ald); (x) alcohol dehydrogenase (Adh); (xi) succinyl CoA synthease beta subunit (SucC); (xii) succinyl CoA synthease alpha subunit (SucD); (xiii) fumarate hydratase (fum); (xiv) fumarate reductase (frd); or (xv) any combination thereof; and (b) growing the microorganism to produce 1,4-BDO, (c) condensing the 1,4-BDO from (b) with a dicarboxylic acid/anhydride to produce a polyurethane. In some cases the dicarboxylic acid/anhydride can be aliphatic or aromatic.

The microorganism can be a microorganism that is capable of converting a C1 carbon into a multicarbon product. For example, a methanotroph can be used. Further any of the genetically modified microorganisms described throughout can be used.

In some instances, the 1,4-BDO can be isolated or purified before proceeding to the condensation process.

Once made, the polyurethanes can be isolated and/or purified. The polyurethanes can be substantially pure.

It should be appreciated that the methods of the invention may be integrated or linked with one or more methods for the production of downstream products from PBT, THF, and/or polyurethanes. For example, the methods described may feed PBT, THF, and/or polyurethanes directly or indirectly to chemical processes or reactions sufficient for the conversion or production of other useful chemical products. In some embodiments, as noted herein before, 1,4-BDO can be converted to one or more chemical products directly via the intermediate compounds PBT, THF, and/or polyurethanes without the need for recovery of PBT, THF, and/or polyurethanes from the method before subsequent use in production of the one or more chemical products. Some commercially relevant products that can be made from 1,4-BDO, PBT, THF, and/or polyurethanes include, but are not limited to, Spandex.

### IV. Fermentation

In general, the microorganisms disclosed herein should be used in fermentation conditions that are appropriate to convert a C1 carbon (such as methane) to 1,4-BDO (or other desired products). Reaction conditions that should be considered include temperature, media flow rate, pH, incubation period, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

The optimum reaction conditions will depend partly on the particular microorganism of used. However, in general, it is preferred that the fermentation be performed at a pressure higher than ambient pressure. Operating at increased pressures allows a significant increase in the rate of C1 carbon transfer from the gas phase to the liquid phase where it can be taken up by the microorganism as a carbon source for the production of 1,4-BDO. This in turn means that the retention time (defined as the liquid volume in the bioreactor divided by the input gas flow rate) can be reduced when bioreactors are maintained at elevated pressure rather than atmospheric pressure.

The use of pressurized systems can greatly reduce the volume of the bioreactor required, and consequently the capital cost of the fermentation equipment. In some cases, reactor volume can be reduced in linear proportion to increases in reactor operating pressure, i.e. bioreactors operated at 10 atmospheres of pressure need only be one tenth the volume of those operated at 1 atmosphere of pressure.

It is also desirable that the rate of introduction of the C1 carbon substrate (such as methane) containing gaseous substrate is such as to ensure that the concentration of the C1 carbon substrate (such as methane) in the liquid phase does not become limiting. This is because a consequence of C1 carbon substrate (e.g., methane) limited conditions may be that the 1,4-BDO product (or other desired product) is consumed by the culture.

### Fermentation Conditions

pH can be optimized based on the microorganism used. For example, the pH used during the methanotroph fermentation of methane to a desired product, can from 4 to 10. In other instances, the pH can be from 5 to 9; 6 to 8; 6.1 to 7.9; 6.2 to 7.8; 6.3 to 7.7; 6.4 to 7.6; or 6.5 to 7.5. For example, the pH can be from 6.6 to 7.4. In some instances, the pH can be from 5 to 9. In some instances, the pH can be from 6 to 8. In some instances, the pH can be from 6.1 to 7.9. In some instances, the pH can be from 6.2 to 7.8. In some instances, the pH can be from 6.3 to 7.7. In some instances, the pH can be from 6.4 to 7.6. In some instances, the pH can be from 6.5 to 7.5. In some instances the pH used for the fermentation of methanotrophs can be greater than 6.

Temperature can also be adjusted based on the microorganism used. For example, the temperature used during the methanotroph fermentation of methane to a desired product, can from 30 C.° to 45 C.°. In other instances, the temperature of the fermentation can be from 30 C.° to 45 C.°; 31 C.° to 44 C.°; 32 C.° to 43 C.°; 33 C.° to 42 C.°; 34 C.° to 41 C.°; 35 C.° to 40 C.°. For example, the temperature can be from 36 C.° to 39 C.° (e.g., 36 C.°, 37 C.°, 38 C.°, or 39) C.°. In some instances, the temperature can be from 30 C.° to 45 C.°. In some instances, the temperature can be from 31 C.° to 44 C.°. In some instances, the temperature can be from 32 C.° to 43 C.°. In some instances, the temperature can be from 33 C.° to 42 C.°. In some instances, the temperature can be from 34 C.° to 41 C.°. In some instances, the temperature can be from 35 C.° to 40 C.°.

Availability of oxygen and other gases such as gaseous C1 carbon substrates (such as methane) can affect yield and fermentation rate. For example, when considering oxygen availability, the percent of dissolved oxygen (DO) within the fermentation media can be from 1% to 40%. In certain instances, the DO concentration can be from 1.5% to 35%; 2% to 30%; 2.5% to 25%; 3% to 20%; 4% to 19%; 5% to 18%; 6% to 17%; 7% to 16%; 8% to 15%; 9% to 14%; 10% to 13%; or 11% to 12%. For example, in some cases the DO concentration can be from 2% to 30%. In other cases, the DO can be from 3% to 20%. In some instances, the DO can be from 4% to 10%. In some cases, the DO can be from 1.5% to 35%. In some cases, the DO can be from 2.5% to 25%. In some cases, the DO can be from 4% to 19%. In some cases, the DO can be from 5% to 18%. In some cases, the DO can be from 6% to 17%. In some cases, the DO can be from 7% to 16%. In some cases, the DO can be from 8% to 15%. In some cases, the DO can be from 9% to 14%. In some cases, the DO can be from 10% to 13%. In some cases, the DO can be from 11% to 12%. In some cases, the DO concentration can be lower than 1%. For example, the DO can be from 0% to 1%.

When using a methanotroph, the type of methane substances can have an effect on yield and fermentation rates. For example, natural gas can be used, which typically has a methane content of above 85% (e.g., above 90%) methane. Other components within natural gas can include but is not limited to ethane, propane, iso-butane, normal-butane, iso-pentane, normal pentane, hexanes plus, nitrogen, carbon dioxide, oxygen, hydrogen, and hydrogen sulfides.

“Pure” methane can be used as well. In these cases, the methane typically comes from a tank. The methane contained within these tanks can range from 90% or greater methane content and the remaining gas are other gases (such as carbon dioxide). For example, gas having a methane content of greater than 90% can be used during the fermentation process. In certain instances, the methane concentration can be greater than 90%; 91%; 92%; 93%; 94%; 95%; 96%; 97%; 98%; 99%; or 99.9%. In some instances, the methane concentration can be 90% methane and 10% are other gases (such as carbon dioxide). In other instances, the methane concentration can be 91% methane and 9% are other gases (such as carbon dioxide). In some instances, the methane concentration can be 92% methane and 8% are other gases (such as carbon dioxide). In some instances, the methane concentration can be 93% methane and 7% are other gases (such as carbon dioxide). In some instances, the methane concentration can be 94% methane and 6% are other gases (such as carbon dioxide). In some instances, the methane concentration can be 95% methane and 5% are other gases (such as carbon dioxide). In other instances, the methane concentration can be 96% methane and 4% are other gases (such as carbon dioxide). In some instances, the methane concentration can be 97% methane and 3% are other gases (such as carbon dioxide). In some instances, the methane concentration can be 98% methane and 2% are other gases (such as carbon dioxide). In some instances, the methane concentration can be 99% methane and 1% are other gases (such as carbon dioxide). In some instances, the methane concentration can be 99.9% methane and 0.1% are other gases (such as carbon dioxide).

The length of incubation can have a significant effect on 1,4-BDO (or other products) titer. For example, the microorganism (such as methanotrophs) may produce minimal or no product before 72 hours. In these cases, incubating the microorganism for longer than 72 hours may be necessary. Thus in some cases, the microorganism disclosed throughout can be fermented for at least 73 hours, 74 hours, 75 hours, 76 hours, 77 hours, 78 hours, 79 hours, 80 hours, 81 hours, 82 hours, 83 hours, 84 hours, 85 hours, 86 hours, 87 hours, 89 hours, 90 hours, 91 hours, 92 hours, 93 hours, 94 hours, 95 hours, 96 hours, 97 hours, 98 hours, 99 hours, 100 hours, 101 hours, 102 hours, 103 hours, 104 hours, 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, 120 hours, 121 hours, 122 hours, 123 hours, 124 hours, 125 hours, 126 hours, 127 hours, 128 hours, 129 hours, 130 hours, 131 hours, 132 hours, 133 hours, 134 hours, 135 hours, or 136 hours. In some cases, the microorganism disclosed throughout can be fermented for at least 84 hours. In some cases, the microorganism disclosed throughout can be fermented for at least 96 hours. In some cases, the microorganism disclosed throughout can be fermented for at least 108 hours. In some cases, the microorganism disclosed throughout can be fermented for at least 120 hours. In some cases, the microorganism disclosed throughout can be fermented for at least 132 hours. In some cases, the microorganism disclosed throughout can be fermented for at least 144 hours. In some cases, the microorganism disclosed throughout can be fermented for at least 156 hours.

### Bioreactor

Fermentation reactions may be carried out in any suitable bioreactor. In some embodiments of the invention, the bioreactor may comprise a first, growth reactor in which the micro-organisms are cultured, and a second, fermentation reactor, to which broth from the growth reactor is fed and in which most of the fermentation product (1,4-BDO, for example) is produced.

### Product Recovery

The fermentation of the microorganisms disclosed herein can produce a fermentation broth comprising a desired product (e.g., 1,4-BDO, THF, PBT, and/or polyurethanes) and/or one or more by-products as well as the microorganisms (e.g., a genetically modified methanotroph), in a nutrient medium.

The microorganisms and the methods herein can produce 1,4-BDO at surprisingly high efficiency, more so than other known 1,4-BDO fermentation processes. For example, the microorganisms and the methods disclosed herein can convert a C1 carbon substrate (such as methane) at a yield of greater than 50%. This means that at least 50% of the C1 carbons within the systems are converted into product, such as 1,4-BDO. In some cases, the conversion of a C1 carbon substrate into 1,4-BDO can be at least 60%, 70%, 80%, 81%, 83%, 84%, 85%, 86%, 87%, 88%, 89%, 90%, 91%, 93%, 94%, 95%, 96%, 97%, 98%, or 99%. In some cases, the conversion of a C1 carbon substrate into 1,4-BDO can be at least 60%. In some cases, the conversion of a C1 carbon substrate into 1,4-BDO can be at least 70%. In some cases, the conversion of a C1 carbon substrate into 1,4-BDO can be at least 80%. In some cases, the conversion of a C1 carbon substrate into 1,4-BDO can be at least 81%. In some cases, the conversion of a C1 carbon substrate into 1,4-BDO can be at least 82%. In some cases, the conversion of a C1 carbon substrate into 1,4-BDO can be at least 83%. In some cases, the conversion of a C1 carbon substrate into 1,4-BDO can be at least 84%. In some cases, the conversion of a C1 carbon substrate into 1,4-BDO can be at least 85%. In some cases, the conversion of a C1 carbon substrate into 1,4-BDO can be at least 86%. In some cases, the conversion of a C1 carbon substrate into 1,4-BDO can be at least 87%. In some cases, the conversion of a C1 carbon substrate into 1,4-BDO can be at least 88%. In some cases, the conversion of a C1 carbon substrate into 1,4-BDO can be at least 89%. In some cases, the conversion of a C1 carbon substrate into 1,4-BDO can be at least 90%. In some cases, the conversion of a C1 carbon substrate into 1,4-BDO can be at least 91%. In some cases, the conversion of a C1 carbon substrate into 1,4-BDO can be at least 92%. In some cases, the conversion of a C1 carbon substrate into 1,4-BDO can be at least 93%. In some cases, the conversion of a C1 carbon substrate into 1,4-BDO can be at least 94%. In some cases, the conversion of a C1 carbon substrate into 1,4-BDO can be at least 95%. In some cases, the conversion of a C1 carbon substrate into 1,4-BDO can be at least 96%. In some cases, the conversion of a C1 carbon substrate into 1,4-BDO can be at least 97%. In some cases, the conversion of a Cl carbon substrate into 1,4-BDO can be at least 98%. In some cases, the conversion of a C1 carbon substrate into 1,4-BDO can be at least 99%.

In certain methods when producing 1,4-BDO, the concentration of 1,4-BDO in the fermentation broth is at least 1 g/L. For example, the concentration of 1,4-BDO produced in the fermentation broth can be from 1 g/L to 5 g/L, 2 g/L to 6 g/L, 3 g/L to 7 g/L, 4 g/L to 8 g/L, 5 g/L to 9 g/L, or 6 g/L to 10 g/L. In some cases, the concentration of 1,4-BDO can be at least 9 g/L. In some cases, the concentration of 1,4-BDO can be from 1 g/L to 5 g/L. In some cases, the concentration of 1,4-BDO can be from 2 g/L to 6 g/L. In some cases, the concentration of 1,4-BDO can be from 3 g/L to 7 g/L. In some cases, the concentration of 1,4-BDO can be from 4 g/L to 8 g/L. In some cases, the concentration of 1,4-BDO can be from 5 g/L to 9 g/L. In some cases, the concentration of 1,4-BDO can be from 6 g/L to 10 g/L.

In other cases, when microorganisms are used that normally produce at least some 1,4-BDO, after genetic modification and fermentation, the genetically modified microorganism can produce 1,4-BDO in concentrations that are at least 1.1× the amount that is normally produced (e.g., without using any genetically modified microorganisms). In some cases, the genetically modified microorganism can produce at least 2×, 3×, 4×, 5×, 10×, 25×, 50×, and or 100× the amount that is normally produced. In some cases, the genetically modified microorganism can produce at least 2× the amount that is normally produced. In some cases, the genetically modified microorganism can produce at least 3× the amount that is normally produced. In some cases, the genetically modified microorganism can produce at least 4× the amount that is normally produced. In some cases, the genetically modified microorganism can produce at least 5× the amount that is normally produced. In some cases, the genetically modified microorganism can produce at least 10× the amount that is normally produced. In some cases, the genetically modified microorganism can produce at least 25× the amount that is normally produced. In some cases, the genetically modified microorganism can produce at least 50× the amount that is normally produced. In some cases, the genetically modified microorganism can produce at least 100× the amount that is normally produced.

As discussed above, in certain embodiments the 1,4-BDO produced in the fermentation reaction is converted to PBT, THF, and/or polyurethane (or other products) directly from the fermentation broth. In other embodiments, the 1,4-BDO is first recovered from the fermentation broth before conversion to PBT, THF, and/or polyurethane.

In some cases, 1,4-BDO can be continuously removed from a portion of broth and recovered as purified 1,4-BDO. In particular embodiments, the recovery of 1,4-BDO includes passing the removed portion of the broth containing 1,4-BDO through a separation unit to separate the microorganisms (e.g., genetically modified methanotroph) from the broth, to produce a cell-free 1,4-BDO containing permeate, and returning the microorganisms to the bioreactor. The cell-free 1,4-BDO-containing permeate may then can be stored or be used for subsequent conversion to PBT, THF, and/or polyurethane (or other desired product).

The recovering of 1,4-BDO and/or one or more other products or by-products produced in the fermentation reaction can comprise continuously removing a portion of the broth and recovering separately 1,4-BDO and one or more other products from the removed portion of the broth. In some embodiments the recovery of 1,4-BDO and/or one or more other products includes passing the removed portion of the broth containing 1,4-BDO and/or one or more other products through a separation unit to separate microorganisms from the 1,4-BDO and/or one or more other products, to produce a cell-free 1,4-BDO and one or more other product-containing permeate, and returning the microorganisms to the bioreactor.

In the above embodiments, the recovery of 1,4-BDO and one or more other products can include first removing 1,4-BDO from the cell-free permeate followed by removing the one or more other products from the cell-free permeate. The cell-free permeate can be then returned to the bioreactor.

1,4-BDO, or a mixed product stream containing 1,4-BDO, can be recovered from the fermentation broth. For example, methods that can be used can include but are not limited to, fractional distillation or evaporation, pervaporation, and extractive fermentation. Further examples include: recovery using steam from whole fermentation broths (Wheat et al. 1948); reverse osmosis combined with distillation (Sridhar 1989); Liquid-liquid extraction techniques involving solvent extraction of 1,4-BDO (Othmer et al. 1945; Tsao 1978; Eiteman and Gainer 1989); aqueous two-phase extraction of 1,4-BDO in PEG/dextran system (Ghosh and Swaminathan 2003; solvent extraction using alcohols or esters, e.g., ethyl acetate, tributylphosphate, diethyl ether, n-butanol, dodecanol, oleyl alcohol, and an ethanol/phosphate system (Bo Jianga 2009); aqueous two-phase systems composed of hydrophilic solvents and inorganic salts (Zhigang et. al. 2010).

Pervaporation or vacuum membrane distillation, used previously in ethanol and butanol fermentations, can be used to concentrate 1,4-BDO (Qureshi et al. 1994) in water as an extract from the fermentation broth. A microporous polytetrafluoroethylene (PTFE) membrane is used in the integrated process, while a silicone membrane is usually used in pervaporative ethanol or butanol fermentations.

By-products such as acids including acetate and butyrate may also be recovered from the fermentation broth using methods known in the art. For example, an adsorption system involving an activated charcoal filter or electrodialysis may be used.

In certain embodiments of the invention, 1,4-BDO and by-products are recovered from the fermentation broth by continuously removing a portion of the broth from the bioreactor, separating microbial cells from the broth (conveniently by filtration, for example), and recovering 1,4-BDO and optionally other alcohols and acids from the broth. Alcohols may conveniently be recovered for example by distillation, and acids may be recovered for example by adsorption on activated charcoal. The separated microbial cells are preferably returned to the fermentation bioreactor. The cell free permeate remaining after the alcohol(s) and acid(s) have been removed is also preferably returned to the fermentation bioreactor. Additional nutrients may be added to the cell free permeate to replenish the nutrient medium before it is returned to the bioreactor.

Also, if the pH of the broth was adjusted during recovery of 1,4-BDO and/or by-products, the pH should be re-adjusted to a similar pH to that of the broth in the fermentation bioreactor, before being returned to the bioreactor.

In certain embodiments, the 1,4-BDO is continuously recovered from the fermentation broth or bioreactor and fed directly for chemical conversion to one or more of THF, PBT, and/or polyurethanes. For example, the 1,4-BDO may be fed directly through a conduit to one or more vessel suitable for chemical synthesis of one or more of THF, PBT, polyurethanes or other down-stream chemical products.

While some embodiments have been shown and described herein, such embodiments are provided by way of example only. Numerous variations, changes, and substitutions will now occur to those skilled in the art without departing from the invention. It should be understood that various alternatives to the embodiments of the invention described herein will be employed in practicing the invention.

### EXAMPLES

### Example 1: Genetic Engineering of a Microorganism to Produce 1,4-BDO Pathway from α-Ketoglutarate

To engineer an α-ketoglutarate to 1,4-BDO pathway (see FIG. 2) in a microorganism, we started with a methanotroph (specifically M. capsulatus) and tested several genes from various bacteria and yeast. Table 1 below shows the genes tested and the origin of the genes.

TABLE 1

| Gene | Gene variants | Gene ID | Protein ID | Organism |
|---|---|---|---|---|
| Kgd | Mbo. Kgd | Mb1280c | Q7U0A6 | Mycobacterium bovis |
| | Mtu. Kgd | Rv1248c | P9WIS5 | Mycobacterium tuberculosis H37Rv |
| | Cte.kgd | A606_07515 | S4XHJ4 | Corynebacterium terpenotabidum Y-11 |
| | Rjo.kgd | RHA1_ro06012 | Q0S3U7 | Rhodococcus jostii (strain RHA1) |
| 4hBD | Eco. Yihu | b3882 | P0A9V8 | Escherichia coli K-12 |
| | Pgi 4hbd | PG0689 | Q7MWD4 | Porphyromonas gingivalis W38 |
| | Ckl 4hbd | CKL_3014 | P38945 | Clostridium kluyveri DSM 555 |
| Cat2 | Pgi abfT-1 | PG0690 | Q7MWD3 | Porphyromonas gingivalis W38 |
| | Pgi abfT-2 | PG1956 | Q7MTJ6 | Porphyromonas gingivalis W38 |
| | Cam abft | abfT | Q9RM86 | Clostridium acetobutylicum |
| Ald/ADH | Cam. adhE2 | CA_P0035 | Q9ANR5 | Clostridium acetobutylicum (strain ATCC 824) |
| | Eco. adhE | b1241 | P0A9Q7 | Escherichia coli K-12 |
| | Eco.yqhD | b3011 | Q46856 | Escherichia coli K-12 |
| | Eco.fucO | b2799 | P0A9S1 | Escherichia coli K-12 |
| | Sce. ADH6 | YMR318C | Q04894 | Saccharomyces cerevisiae |
| | Aba. ADP1 | ACIAD3612 | Q6F6R9 | Acinetobacter baylyi (strain ATCC 33305) |
| | Eco. ahr (yjgB) | b4269 | P27250 | Escherichia coli K-12 |

Several strains were generated (as seen in Table 2 below) and cultured in cell culture media supplemented with exogenous α-ketoglutarate. Strains were cultured in microtiter plates for a total of 7 days at 37° C. Of this total 7 days, 3 days were done as a pre-induction and 4 days after induction. The strains were tested for γ-hydroxybutyrate (“GHB”) and 1,4-BDO formation. Table 2 below shows the various production amounts of the different strains. Strain XZ79 produced both the highest amounts of GHB and 1,4-BDO. This strain expressed a Mycobacterium bovis Kgd, Clostridium kluyveri 4hbD, Porphyromonas gingivalis Cat2, Clostridium acetobutylicum Ald and an Acinetobacter baylyi Adh.

TABLE 2

| | GHB | 1,4 BDO | Genotype | | | | |
|---|---|---|---|---|---|---|---|
| Sample | (μg/L) | (μg/L) | kgd | 4hbD | Cat2 | Ald | Adh |
| XZ76 | 209 | 118 | g.Mbo.SucA | g.Eco.yihu | g.Pgi.Cat2.abfT-1 | g.Cac.adhe2 | g.Aba.ADP1 |
| XZ79 | 875 | 261 | g.Mbo.SucA | g.Ckl.4hbD | g.Pgi.Cat2.abfT-1 | g.Cac.adhe2 | g.Aba.ADP1 |
| XZ84 | 461 | | g.Mbo.SucA | g.Eco.yihu | g.Cam.abft | g.Cac.adhe2 | g.Aba.ADP1 |
| XZ276 | 535 | | g.Mbo.SucA | g.Eco.yihu | g.Pgi.Cat2.abfT-1 | g.Eco.adhe | g.Aba.ADP1 |
| XZ88 | 530 | | g.Mbo.SucA | g.Eco.yihu | g.Pgi.Cat2.abfT-1 | g.Eco.fucO | g.Aba.ADP1 |
| XZ278 | 163 | 52 | g.Mbo.SucA | g.Eco.yihu | g.Pgi.Cat2.abfT-1 | g.Cac.adhe2 | g.Sce.adh6 |
| XZ95 | 305 | | g.Mbo.SucA | g.Eco.yihu | g.Pgi.Cat2.abfT-1 | g.Eco.yqhD | g.Sce.adh6 |

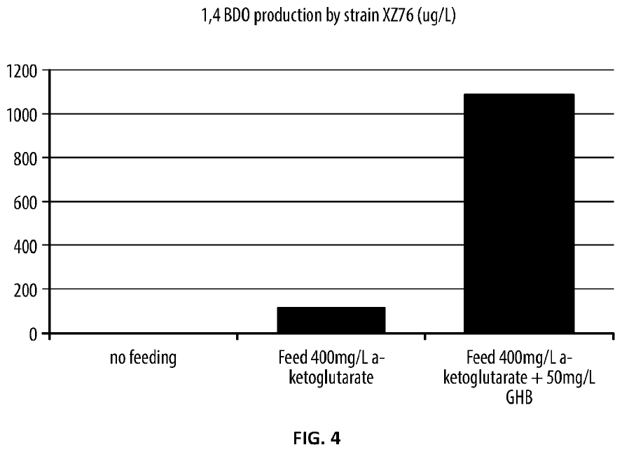

Strain XZ76 was further tested. This strain was cultured in the same media and time frame as above however supplemented with exogenous α-ketoglutarate (400 mg/L) and exogenous GHB (50 mg/L). 1,4-BDO production by these strains were tested. As shown in FIG. 4, the production of 1,4-BDO was seen with α-ketoglutarate alone to a level greater than 100 ug/L. Surprisingly, however, when the culture media was supplemented with exogenous GHB, 1,4-BDO production spiked approximately 11-fold over α-ketoglutarate alone.

### Example 2: Genetic Engineering of a Microorganism to Produce 1,4-BDO from Succinate

Methanotrophs were further genetically engineered to express enzymes of the succinate to 1,4-BDO pathway. Methanotrophs were transformed with various genes from various bacteria and yeast. Table 3 below shows the genes tested and the origin of the genes.

TABLE 3

| Gene | Gene variants | Gene ID | Protein ID | Organism |
|---|---|---|---|---|
| SucC/SucD | Eco.SucC | b0728 | P0A836 | Escherichia coli K-12 |
| | Eco.SucD | b0729 | P0AGE9 | Escherichia coli K-12 |
| | Ckl.SucD | CKL_3015 | P38947 | Clostridium kluyveri DSM 555 |
| | Pgi.SucD | PG0687 | Q7MWD5 | Porphyromonas gingivalis W38 |
| 4hBD | Eco. Yihu | b3882 | P0A9V8 | Escherichia coli K-12 |
| | Pgi 4hbd | PG0689 | Q7MWD4 | Porphyromonas gingivalis W38 |
| | Ckl 4hbd | CKL_3014 | P38945 | Clostridium kluyveri DSM 555 |
| Cat2 | Pgi abfT-1 | PG0690 | Q7MWD3 | Porphyromonas gingivalis W38 |
| | Pgi abfT-2 | PG1956 | Q7MTJ6 | Porphyromonas gingivalis W38 |
| | Cam abft | abfT | Q9RM86 | Clostridium acetobutylicum |
| Ald/ADH | Cam. adhE2 | CA_P0035 | Q9ANR5 | Clostridium acetobutylicum (strain ATCC 824) |
| | Eco. adhE | b1241 | P0A9Q7 | Escherichia coli K-12 |
| | Eco.yqhD | b3011 | Q46856 | Escherichia coli K-12 |
| | Eco.fucO | b2799 | P0A9S1 | Escherichia coli K-12 |
| | Sce. ADH6 | YMR318C | Q04894 | Saccharomyces cerevisiae |
| | Aba. ADP1 | ACIAD3612 | Q6F6R9 | Acinetobacter baylyi (strain ATCC 33305) |
| | Eco. ahr (yjgB) | b4269 | P27250 | Escherichia coli K-12 |

Several strains were generated (as seen in Table 4 below) and cultured in IM5 media supplemented with exogenous succinic acid. Strains were cultured in microtiter plates and for the same period of time (7 days: 3 days pre-induction and 4 days of induction at 37° C.) and tested for GHB and 1,4-BDO formation. Table 4 below shows the various production amounts of the different strains. Strain XZ440 produced the highest amounts of GHB. Strain XZ440 expressed an Escherichia coli SucD; Porphyromonas gingivalis SucD; Porphyromonas gingivalis 4hbD; Porphyromonas gingivalis Cat2.abfT-2, Clostridium acetobutylicum adhe2; and Acinetobacter baylyi ADP1. Strain XZA38 produced the highest amounts of 1,4-BDO. This strain expressed an Escherichia coli SucD; Porphyromonas gingivalis SucD; Porphyromonas gingivalis 4hbD; Porphyromonas gingivalis Cat2.abfT-1, Clostridium acetobutylicum adhe2; and Acinetobacter baylyi ADP1.

TABLE 4

| | GHB | 1,4-BDO | | | | | | |
|---|---|---|---|---|---|---|---|---|
| Strains | (μg/L) | (μg/L) | Genotype | | | | | |
| XZ344 | 33.608 | | g.Eco.SucD | g.Pgi.SucD | g.Pgi.4hbD | g.Pgi.Cat2.abfT-1 | g.Cac.adhe2 | g.Aba.ADP1 |
| XZ345 | 13455.08 | | g.Eco.SucD | g.Pgi.SucD | g.Pgi.4hbD | g.Pgi.Cat2.abfT-1 | g.Cac.adhe2 | g.Aba.ADP1 |
| XZ347 | 3434.5 | | g.Eco.SucD | g.Pgi.SucD | g.Pgi.4hbD | g.Pgi.Cat2.abfT-2 | g.Cac.adhe2 | g.Aba.ADP1 |
| XZ350 | 201.916 | | g.Eco.SucC | g.Ckl.SucD | g.Eco.yihu | g.Pgi.Cat2.abfT-2 | g.Eco.adhe | g.Eco.ahr |
| XZ437 | 16029.96 | | g.Eco.SucC | g.Pgi.SucD | g.Pgi.4hbD | g.Pgi.Cat2.abfT-2 | g.Cac.adhe2 | g.Aba.ADP1 |
| XZ438 | 3236.62 | 73.018 | g.Eco.SucD | g.Pgi.SucD | g.Pgi.4hbD | g.Pgi.Cat2.abfT-1 | g.Cac.adhe2 | g.Aba.ADP1 |
| XZ439 | 58.593 | | g.Eco.SucD | g.Pgi.SucD | g.Pgi.4hbD | g.Pgi.Cat2.abfT-1 | g.Cac.adhe2 | g.Aba.ADP1 |
| XZ440 | 16707.54 | | g.Eco.SucD | g.Pgi.SucD | g.Pgi.4hbD | g.Pgi.Cat2.abfT-2 | g.Cac.adhe2 | g.Aba.ADP1 |
| XZ441 | 1190.559 | | g.Eco.SucD | g.Pgi.SucD | g.Pgi.4hbD | g.Pgi.Cat2.abfT-2 | g.Cac.adhe2 | g.Aba.ADP1 |
| XZ442 | 5355.94 | | g.Eco.SucD | g.Pgi.SucD | g.Pgi.4hbD | g.Pgi.Cat2.abfT-1 | g.Eco.adhe | g.Eco.ahr |
| XZ443 | 3442.24 | | g.Eco.SucD | g.Pgi.SucD | g.Pgi.4hbD | g.Pgi.Cat2.abfT-1 | g.Eco.adhe | g.Eco.ahr |

### Example 3: Genetic Engineering to Produce Higher Levels of α-Ketoglutarate

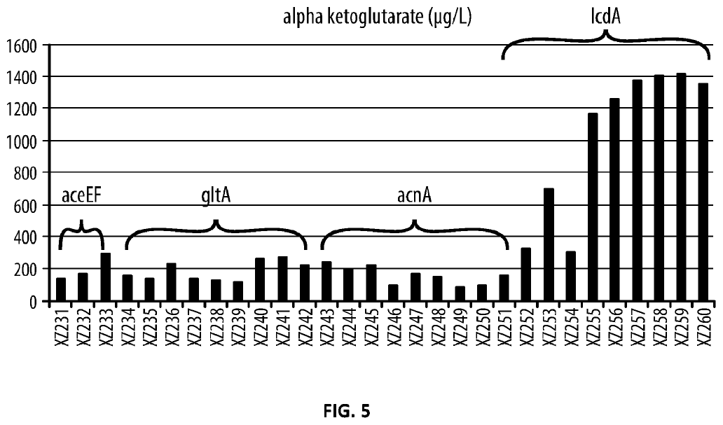

After finding that 1,4-BDO could be produced in surprisingly high quantities and very efficiently, we focused our attention to the pyruvate to the α-ketoglutarate part of the pathway. FIG. 1 (with the circle) shows the pathway from pyruvate to the α-ketoglutarate. We attempted to increase the overall amount of α-ketoglutarate produced in our system. To do this, we overexpressed four genes in the pathway: aceEF, gltA, acnA, or IcdA. FIG. 5 shows the results of this study.

Overexpression of aceEF, gltA, and acnA lead to only minor production of α-ketoglutarate. However, overexpression of IcdA (isocitrate dehydrogenase) significantly increased the overall α-ketoglutarate levels. The highest producer of α-ketoglutarate was Strain XZ259 with α-ketoglutarate at levels of over 1400 ug/L.

### Example 4:1,4-BDO Production Directly from Methane in Microtiter Plates

We generated 78 and 48 different broad host plasmids containing variations in the 5-gene 1,4 BDO pathway, from the α-ketoglutarate and succinate pathway respectively, and transformed the aforementioned plasmids into a transformation competent methanotroph strain, RL83A, and evaluated 112 and 60 resulting strains (including biological replicate strains) for 1,4 BDO production in small scale microtiter plate fermentation using methane as the carbon source. For these experiments, starter cultures were inoculated into microtiter plates containing 500 μl of media. The microtiter plates were fed a source of methane, arabinose, ketoglutarate or succinate, and incubated in a shaker at 37° C. for 96 hours. Samples were tested after 96 hours.

The genotypes for each of the strains are provided in Table 5 (α-ketoglutarate pathway) and Table 6 (succinate pathway) below.

TABLE 5
Genotypes of Microorganisms tested for the α-ketoglutarate pathway

| Plasmid | Strain | Heterologous genes | | | | |
|---|---|---|---|---|---|---|
| p172asn1 | XZ75 | g.Mbo.SucA | g.Eco.yihu | g.Pgi.Cat2.abfT-1 | g.Cac.adhe2 | g.Aba.ADP1 |
| p172asn1 | XZ76 | g.Mbo.SucA | g.Eco.yihu | g.Pgi.Cat2.abfT-1 | g.Cac.adhe2 | g.Aba.ADP1 |
| p172asn2 | XZ77 | g.Mbo.SucA | g.Pgi.4hbD | g.Pgi.Cat2.abfT-1 | g.Cac.adhe2 | g.Aba.ADP1 |
| p172asn2 | XZ78 | g.Mbo.SucA | g.Pgi.4hbD | g.Pgi.Cat2.abfT-1 | g.Cac.adhe2 | g.Aba.ADP1 |
| p172asn3 | XZ79 | g.Mbo.SucA | g.Ckl.4hbD | g.Pgi.Cat2.abfT-1 | g.Cac.adhe2 | g.Aba.ADP1 |
| p172asn3 | XZ80 | g.Mbo.SucA | g.Ckl.4hbD | g.Pgi.Cat2.abfT-1 | g.Cac.adhe2 | g.Aba.ADP1 |
| p172asn4 | XZ81 | g.Mbo.SucA | g.Eco.yihu | g.Pgi.Cat2.abfT-2 | g.Cac.adhe2 | g.Aba.ADP1 |
| p172asn4 | XZ82 | g.Mbo.SucA | g.Eco.yihu | g.Pgi.Cat2.abfT-2 | g.Cac.adhe2 | g.Aba.ADP1 |
| p172asn5 | XZ83 | g.Mbo.SucA | g.Eco.yihu | g.Cam.abft | g.Cac.adhe2 | g.Aba.ADP1 |
| p172asn5 | XZ84 | g.Mbo.SucA | g.Eco.yihu | g.Cam.abft | g.Cac.adhe2 | g.Aba.ADP1 |
| p172asn6 | XZ276 | g.Mbo.SucA | g.Eco.yihu | g.Pgi.Cat2.abfT-1 | g.Eco.adhe | g.Aba.ADP1 |
| p172asn6 | XZ277 | g.Mbo.SucA | g.Eco.yihu | g.Pgi.Cat2.abfT-1 | g.Eco.adhe | g.Aba.ADP1 |
| p172asn7 | XZ85 | g.Mbo.SucA | g.Eco.yihu | g.Pgi.Cat2.abfT-1 | g.Eco.yqhD | g.Aba.ADP1 |
| p172asn7 | XZ86 | g.Mbo.SucA | g.Eco.yihu | g.Pgi.Cat2.abfT-1 | g.Eco.yqhD | g.Aba.ADP1 |
| p172asn8 | XZ87 | g.Mbo.SucA | g.Eco.yihu | g.Pgi.Cat2.abfT-1 | g.Eco.fucO | g.Aba.ADP1 |
| p172asn8 | XZ88 | g.Mbo.SucA | g.Eco.yihu | g.Pgi.Cat2.abfT-1 | g.Eco.fucO | g.Aba.ADP1 |
| p172asn9 | XZ278 | g.Mbo.SucA | g.Eco.yihu | g.Pgi.Cat2.abfT-1 | g.Cac.adhe2 | g.Sce.adh6 |
| p172asn9 | XZ279 | g.Mbo.SucA | g.Eco.yihu | g.Pgi.Cat2.abfT-1 | g.Cac.adhe2 | g.Sce.adh6 |
| p172asn10 | XZ280 | g.Mbo.SucA | g.Eco.yihu | g.Pgi.Cat2.abfT-1 | g.Cac.adhe2 | g.Eco.ahr |
| p172asn10 | XZ281 | g.Mbo.SucA | g.Eco.yihu | g.Pgi.Cat2.abfT-1 | g.Cac.adhe2 | g.Eco.ahr |
| p172asn11 | XZ89 | g.Mtu.kgd | g.Eco.yihu | g.Pgi.Cat2.abfT-1 | g.Cac.adhe2 | g.Aba.ADP1 |
| p172asn12 | XZ90 | g.Cte.kgd | g.Eco.yihu | g.Pgi.Cat2.abfT-1 | g.Cac.adhe2 | g.Aba.ADP1 |
| p172asn12 | XZ91 | g.Cte.kgd | g.Eco.yihu | g.Pgi.Cat2.abfT-1 | g.Cac.adhe2 | g.Aba.ADP1 |
| p172asn13 | XZ92 | g.Rjo.odhA | g.Eco.yihu | g.Pgi.Cat2.abfT-1 | g.Cbe.Ald | g.Aba.ADP1 |
| p172asn13 | XZ93 | g.Rjo.odhA | g.Eco.yihu | g.Pgi.Cat2.abfT-1 | g.Cbe.Ald | g.Aba.ADP1 |
| p172asn14 | XZ94 | g.Mbo.SucA | g.Eco.yihu | g.Pgi.Cat2.abfT-1 | g.Eco.yqhD | g.Sce.adh6 |
| p172asn14 | XZ95 | g.Mbo.SucA | g.Eco.yihu | g.Pgi.Cat2.abfT-1 | g.Eco.yqhD | g.Sce.adh6 |
| p172asn15 | XZ96 | g.Mbo.SucA | g.Eco.yihu | g.Pgi.Cat2.abfT-1 | g.Eco.fucO | g.Sce.adh6 |
| p172asn16 | XZ97 | g.Mbo.SucA | g.Eco.yihu | g.Pgi.Cat2.abfT-1 | g.Eco.adhe | g.Sce.adh6 |
| p172asn16 | XZ98 | g.Mbo.SucA | g.Eco.yihu | g.Pgi.Cat2.abfT-1 | g.Eco.adhe | g.Sce.adh6 |
| p172asn17 | XZ99 | g.Mbo.SucA | g.Eco.yihu | g.Pgi.Cat2.abfT-2 | g.Eco.adhe | g.Eco.ahr |
| p172asn17 | XZ100 | g.Mbo.SucA | g.Eco.yihu | g.Pgi.Cat2.abfT-2 | g.Eco.adhe | g.Eco.ahr |
| p172asn18 | XZ216 | g.Mbo.SucA | g.Pgi.4hbD | g.Pgi.Cat2.abfT-2 | g.Eco.adhe | g.Sce.adh6 |
| p172asn18 | XZ217 | g.Mbo.SucA | g.Pgi.4hbD | g.Pgi.Cat2.abfT-2 | g.Eco.adhe | g.Sce.adh6 |
| p172asn20 | XZ101 | g.Mbo.SucA | g.Pgi.4hbD | g.Pgi.Cat2.abfT-1 | g.Cac.adhe2 | g.Aba.ADP1 |
| p172asn20 | XZ102 | g.Mbo.SucA | g.Pgi.4hbD | g.Pgi.Cat2.abfT-1 | g.Cac.adhe2 | g.Aba.ADP1 |
| p172asn21 | XZ282 | g.Mbo.SucA | g.Ckl.4hbD | g.Pgi.Cat2.abfT-1 | g.Cac.adhe2 | g.Aba.ADP1 |
| p172asn24 | XZ283 | g.Mbo.SucA | g.Eco.yihu | g.Pgi.Cat2.abfT-1 | g.Eco.adhe | g.Aba.ADP1 |
| p172asn24 | XZ284 | g.Mbo.SucA | g.Eco.yihu | g.Pgi.Cat2.abfT-1 | g.Eco.adhe | g.Aba.ADP1 |
| p172asn25 | XZ285 | g.Mbo.SucA | g.Eco.yihu | g.Pgi.Cat2.abfT-1 | g.Eco.yqhD | g.Aba.ADP1 |
| p172asn25 | XZ286 | g.Mbo.SucA | g.Eco.yihu | g.Pgi.Cat2.abfT-1 | g.Eco.yqhD | g.Aba.ADP1 |
| p172asn26 | XZ103 | g.Mbo.SucA | g.Eco.yihu | g.Pgi.Cat2.abfT-1 | g.Eco.fucO | g.Aba.ADP1 |
| p172asn26 | XZ104 | g.Mbo.SucA | g.Eco.yihu | g.Pgi.Cat2.abfT-1 | g.Eco.fucO | g.Aba.ADP1 |
| p172asn28 | XZ287 | g.Mbo.SucA | g.Eco.yihu | g.Pgi.Cat2.abfT-1 | g.Cac.adhe2 | g.Eco.ahr |
| p172asn28 | XZ288 | g.Mbo.SucA | g.Eco.yihu | g.Pgi.Cat2.abfT-1 | g.Cac.adhe2 | g.Eco.ahr |
| p172asn29 | XZ289 | g.Mtu.kgd | g.Eco.yihu | g.Pgi.Cat2.abfT-1 | g.Cac.adhe2 | g.Aba.ADP1 |
| p172asn29 | XZ290 | g.Mtu.kgd | g.Eco.yihu | g.Pgi.Cat2.abfT-1 | g.Cac.adhe2 | g.Aba.ADP1 |
| p172asn30 | XZ291 | g.Cte.kgd | g.Eco.yihu | g.Pgi.Cat2.abfT-1 | g.Cac.adhe2 | g.Aba.ADP1 |
| p172asn31 | XZ105 | g.Rjo.odhA | g.Eco.yihu | g.Pgi.Cat2.abfT-1 | g.Cbe.Ald | g.Aba.ADP1 |
| p172asn31 | XZ106 | g.Rjo.odhA | g.Eco.yihu | g.Pgi.Cat2.abfT-1 | g.Cbe.Ald | g.Aba.ADP1 |
| p172asn32 | XZ107 | g.Mbo.SucA | g.Eco.yihu | g.Pgi.Cat2.abfT-1 | g.Eco.yqhD | g.Sce.adh6 |
| p172asn32 | XZ108 | g.Mbo.SucA | g.Eco.yihu | g.Pgi.Cat2.abfT-1 | g.Eco.yqhD | g.Sce.adh6 |
| p172asn33 | XZ292 | g.Mbo.SucA | g.Eco.yihu | g.Pgi.Cat2.abfT-1 | g.Eco.fucO | g.Sce.adh6 |
| p172asn33 | XZ293 | g.Mbo.SucA | g.Eco.yihu | g.Pgi.Cat2.abfT-1 | g.Eco.fucO | g.Sce.adh6 |
| p172asn34 | XZ294 | g.Mbo.SucA | g.Eco.yihu | g.Pgi.Cat2.abfT-1 | g.Eco.adhe | g.Sce.adh6 |
| p172asn34 | XZ295 | g.Mbo.SucA | g.Eco.yihu | g.Pgi.Cat2.abfT-1 | g.Eco.adhe | g.Sce.adh6 |
| p172asn35 | XZ109 | g.Mbo.SucA | g.Eco.yihu | g.Pgi.Cat2.abfT-2 | g.Eco.adhe | g.Eco.ahr |
| p172asn36 | XZ110 | g.Mbo.SucA | g.Pgi.4hbD | g.Pgi.Cat2.abfT-2 | g.Eco.adhe | g.Sce.adh6 |
| p172asn36 | XZ296 | g.Mbo.SucA | g.Pgi.4hbD | g.Pgi.Cat2.abfT-2 | g.Eco.adhe | g.Sce.adh6 |
| p172asn36 | XZ297 | g.Mbo.SucA | g.Pgi.4hbD | g.Pgi.Cat2.abfT-2 | g.Eco.adhe | g.Sce.adh6 |
| p172asn37 | XZ111 | g.Eco.yihu | g.Mbo.SucA | g.Pgi.Cat2.abfT-1 | g.Cac.adhe2 | g.Aba.ADP1 |
| p172asn37 | XZ112 | g.Eco.yihu | g.Mbo.SucA | g.Pgi.Cat2.abfT-1 | g.Cac.adhe2 | g.Aba.ADP1 |
| p172asn37 | XZ298 | g.Eco.yihu | g.Mbo.SucA | g.Pgi.Cat2.abfT-1 | g.Cac.adhe2 | g.Aba.ADP1 |
| p172asn37 | XZ299 | g.Eco.yihu | g.Mbo.SucA | g.Pgi.Cat2.abfT-1 | g.Cac.adhe2 | g.Aba.ADP1 |
| p172asn37 | XZ300 | g.Eco.yihu | g.Mbo.SucA | g.Pgi.Cat2.abfT-1 | g.Cac.adhe2 | g.Aba.ADP1 |
| p172asn37 | XZ301 | g.Eco.yihu | g.Mbo.SucA | g.Pgi.Cat2.abfT-1 | g.Cac.adhe2 | g.Aba.ADP1 |
| p172asn38 | XZ302 | g.Pgi.4hbD | g.Mbo.SucA | g.Pgi.Cat2.abfT-1 | g.Cac.adhe2 | g.Aba.ADP1 |
| p172asn40 | XZ303 | g.Pgi.4hbD | g.Mbo.SucA | g.Pgi.Cat2.abfT-2 | g.Cac.adhe2 | g.Aba.ADP1 |
| p172asn40 | XZ304 | g.Pgi.4hbD | g.Mbo.SucA | g.Pgi.Cat2.abfT-2 | g.Cac.adhe2 | g.Aba.ADP1 |
| p172asn41 | XZ218 | g.Eco.yihu | g.Mbo.SucA | g.Cam.abft | g.Cac.adhe2 | g.Aba.ADP1 |
| p172asn41 | XZ219 | g.Eco.yihu | g.Mbo.SucA | g.Cam.abft | g.Cac.adhe2 | g.Aba.ADP1 |
| p172asn45 | XZ305 | g.Eco.yihu | g.Mbo.SucA | g.Pgi.Cat2.abfT-2 | g.Cac.adhe2 | g.Sce.adh6 |
| p172asn45 | XZ306 | g.Eco.yihu | g.Mbo.SucA | g.Pgi.Cat2.abfT-2 | g.Cac.adhe2 | g.Sce.adh6 |
| p172asn46 | XZ220 | g.Pgi.4hbD | g.Mbo.SucA | g.Pgi.Cat2.abfT-2 | g.Cac.adhe2 | g.Sce.adh6 |
| p172asn46 | XZ221 | g.Pgi.4hbD | g.Mbo.SucA | g.Pgi.Cat2.abfT-2 | g.Cac.adhe2 | g.Sce.adh6 |
| p172asn47 | XZ307 | g.Eco.yihu | g.Mbo.SucA | g.Cam.abft | g.Cac.adhe2 | g.Sce.adh6 |
| p172asn47 | XZ308 | g.Eco.yihu | g.Mbo.SucA | g.Cam.abft | g.Cac.adhe2 | g.Sce.adh6 |
| p172asn48 | XZ222 | g.Pgi.4hbD | g.Mbo.SucA | g.Cam.abft | g.Cac.adhe2 | g.Sce.adh6 |
| p172asn48 | XZ223 | g.Pgi.4hbD | g.Mbo.SucA | g.Cam.abft | g.Cac.adhe2 | g.Sce.adh6 |
| p172asn49 | XZ309 | g.Eco.yihu | g.Mtu.kgd | g.Pgi.Cat2.abfT-1 | g.Cac.adhe2 | g.Aba.ADP1 |
| p172asn49 | XZ310 | g.Eco.yihu | g.Mtu.kgd | g.Pgi.Cat2.abfT-1 | g.Cac.adhe2 | g.Aba.ADP1 |
| p172asn50 | XZ311 | g.Eco.yihu | g.Cte.kgd | g.Pgi.Cat2.abfT-1 | g.Cac.adhe2 | g.Aba.ADP1 |
| p172asn50 | XZ312 | g.Eco.yihu | g.Cte.kgd | g.Pgi.Cat2.abfT-1 | g.Cac.adhe2 | g.Aba.ADP1 |
| p172asn52 | XZ224 | g.Eco.yihu | g.Mbo.SucA | g.Pgi.Cat2.abfT-1 | g.Eco.adhe | g.Aba.ADP1 |
| p172asn54 | XZ313 | g.Eco.yihu | g.Mbo.SucA | g.Pgi.Cat2.abfT-1 | g.Eco.adhe | g.Eco.ahr |
| p172asn57 | XZ314 | g.Ckl.4hbD | g.Mbo.SucA | g.Pgi.Cat2.abfT-2 | g.Eco.adhe | g.Aba.ADP1 |
| p172asn58 | XZ113 | g.Eco.yihu | g.Mtu.kgd | g.Pgi.Cat2.abfT-2 | g.Eco.adhe | g.Aba.ADP1 |
| p172asn59 | XZ315 | g.Eco.yihu | g.Mbo.SucA | g.Pgi.Cat2.abfT-2 | g.Eco.adhe | g.Sce.adh6 |
| p172asn59 | XZ316 | g.Eco.yihu | g.Mbo.SucA | g.Pgi.Cat2.abfT-2 | g.Eco.adhe | g.Sce.adh6 |
| p172asn60 | XZ225 | g.Eco.yihu | g.Mbo.SucA | g.Pgi.Cat2.abfT-2 | g.Eco.adhe | g.Eco.ahr |
| p172asn60 | XZ226 | g.Eco.yihu | g.Mbo.SucA | g.Pgi.Cat2.abfT-2 | g.Eco.adhe | g.Eco.ahr |
| p172asn61 | XZ114 | g.Mbo.SucA | g.Eco.yihu | g.Pgi.Cat2.abfT-1 | g.Cac.adhe2 | g.Aba.ADP1 |
| p172asn61 | XZ317 | g.Mbo.SucA | g.Eco.yihu | g.Pgi.Cat2.abfT-1 | g.Cac.adhe2 | g.Aba.ADP1 |
| p172asn61 | XZ318 | g.Mbo.SucA | g.Eco.yihu | g.Pgi.Cat2.abfT-1 | g.Cac.adhe2 | g.Aba.ADP1 |
| p172asn62 | XZ319 | g.Mbo.SucA | g.Pgi.4hbD | g.Pgi.Cat2.abfT-1 | g.Cac.adhe2 | g.Aba.ADP1 |
| p172asn62 | XZ320 | g.Mbo.SucA | g.Pgi.4hbD | g.Pgi.Cat2.abfT-1 | g.Cac.adhe2 | g.Aba.ADP1 |
| p172asn64 | XZ321 | g.Mbo.SucA | g.Eco.yihu | g.Pgi.Cat2.abfT-2 | g.Cac.adhe2 | g.Aba.ADP1 |
| p172asn64 | XZ322 | g.Mbo.SucA | g.Eco.yihu | g.Pgi.Cat2.abfT-2 | g.Cac.adhe2 | g.Aba.ADP1 |
| p172asn65 | XZ323 | g.Mbo.SucA | g.Eco.yihu | g.Cam.abft | g.Cac.adhe2 | g.Aba.ADP1 |
| p172asn65 | XZ324 | g.Mbo.SucA | g.Eco.yihu | g.Cam.abft | g.Cac.adhe2 | g.Aba.ADP1 |
| p172asn66 | XZ115 | g.Mbo.SucA | g.Eco.yihu | g.Pgi.Cat2.abfT-1 | g.Eco.adhe | g.Aba.ADP1 |
| p172asn69 | XZ325 | g.Mbo.SucA | g.Eco.yihu | g.Pgi.Cat2.abfT-1 | g.Cac.adhe2 | g.Sce.adh6 |
| p172asn69 | XZ326 | g.Mbo.SucA | g.Eco.yihu | g.Pgi.Cat2.abfT-1 | g.Cac.adhe2 | g.Sce.adh6 |
| p172asn70 | XZ116 | g.Mbo.SucA | g.Eco.yihu | g.Pgi.Cat2.abfT-1 | g.Cac.adhe2 | g.Eco.ahr |
| p172asn70 | XZ117 | g.Mbo.SucA | g.Eco.yihu | g.Pgi.Cat2.abfT-1 | g.Cac.adhe2 | g.Eco.ahr |
| p172asn71 | XZ327 | g.Mtu.kgd | g.Eco.yihu | g.Pgi.Cat2.abfT-1 | g.Cac.adhe2 | g.Aba.ADP1 |
| p172asn71 | XZ328 | g.Mtu.kgd | g.Eco.yihu | g.Pgi.Cat2.abfT-1 | g.Cac.adhe2 | g.Aba.ADP1 |
| p172asn72 | XZ329 | g.Cte.kgd | g.Eco.yihu | g.Pgi.Cat2.abfT-1 | g.Cac.adhe2 | g.Aba.ADP1 |
| p172asn75 | XZ227 | g.Mbo.SucA | g.Eco.yihu | g.Pgi.Cat2.abfT-1 | g.Eco.fucO | g.Sce.adh6 |
| p172asn75 | XZ228 | g.Mbo.SucA | g.Eco.yihu | g.Pgi.Cat2.abfT-1 | g.Eco.fucO | g.Sce.adh6 |
| p172asn77 | XZ229 | g.Mbo.SucA | g.Eco.yihu | g.Pgi.Cat2.abfT-2 | g.Eco.adhe | g.Eco.ahr |
| p172asn77 | XZ230 | g.Mbo.SucA | g.Eco.yihu | g.Pgi.Cat2.abfT-2 | g.Eco.adhe | g.Eco.ahr |
| p172asn77 | XZ330 | g.Mbo.SucA | g.Eco.yihu | g.Pgi.Cat2.abfT-2 | g.Eco.adhe | g.Eco.ahr |
| p172asn77 | XZ331 | g.Mbo.SucA | g.Eco.yihu | g.Pgi.Cat2.abfT-2 | g.Eco.adhe | g.Eco.ahr |
| p172asn78 | XZ332 | g.Mbo.SucA | g.Pgi.4hbD | g.Pgi.Cat2.abfT-2 | g.Eco.adhe | g.Sce.adh6 |
| p172asn78 | XZ333 | g.Mbo.SucA | g.Pgi.4hbD | g.Pgi.Cat2.abfT-2 | g.Eco.adhe | g.Sce.adh6 |

TABLE 6
Genotypes of Microorganisms tested for the Succinate pathway

| Plasmid | Strain | Heterologous genes | | | | | |
|---|---|---|---|---|---|---|---|
| P176-Asn1 | XZ334 | g.Eco.SucC | g.Ckl.SucD | g.Eco.yihu | g.Pgi.Cat2.abfT-1 | g.Eco.adhe | g.Eco.ahr |
| P176-Asn2 | XZ335 | g.Eco.SucC | g.Ckl.SucD | g.Eco.yihu | g.Pgi.Cat2.abfT-2 | g.Eco.adhe | g.Eco.ahr |
| P176-Asn2 | XZ430 | g.Eco.SucC | g.Ckl.SucD | g.Eco.yihu | g.Pgi.Cat2.abfT-2 | g.Eco.adhe | g.Eco.ahr |
| P176-Asn2 | XZ431 | g.Eco.SucC | g.Ckl.SucD | g.Eco.yihu | g.Pgi.Cat2.abfT-2 | g.Eco.adhe | g.Eco.ahr |
| P176-Asn3 | XZ336 | g.Eco.SucD | g.Ckl.SucD | g.Eco.yihu | g.Pgi.Cat2.abfT-1 | g.Eco.adhe | g.Eco.ahr |
| P176-Asn3 | XZ337 | g.Eco.SucD | g.Ckl.SucD | g.Eco.yihu | g.Pgi.Cat2.abfT-1 | g.Eco.adhe | g.Eco.ahr |
| P176-Asn4 | XZ338 | g.Eco.SucD | g.Ckl.SucD | g.Eco.yihu | g.Pgi.Cat2.abfT-2 | g.Eco.adhe | g.Eco.ahr |
| P176-Asn4 | XZ339 | g.Eco.SucD | g.Ckl.SucD | g.Eco.yihu | g.Pgi.Cat2.abfT-2 | g.Eco.adhe | g.Eco.ahr |
| P176-Asn4 | XZ432 | g.Eco.SucD | g.Ckl.SucD | g.Eco.yihu | g.Pgi.Cat2.abfT-2 | g.Eco.adhe | g.Eco.ahr |
| P176-Asn4 | XZ433 | g.Eco.SucD | g.Ckl.SucD | g.Eco.yihu | g.Pgi.Cat2.abfT-2 | g.Eco.adhe | g.Eco.ahr |
| P176-Asn5 | XZ340 | g.Eco.SucC | g.Pgi.SucD | g.Pgi.4hbD | g.Pgi.Cat2.abfT-1 | g.Cac.adhe2 | g.Aba.ADP1 |
| P176-Asn5 | XZ341 | g.Eco.SucC | g.Pgi.SucD | g.Pgi.4hbD | g.Pgi.Cat2.abfT-1 | g.Cac.adhe2 | g.Aba.ADP1 |
| P176-Asn5 | XZ434 | g.Eco.SucC | g.Pgi.SucD | g.Pgi.4hbD | g.Pgi.Cat2.abfT-1 | g.Cac.adhe2 | g.Aba.ADP1 |
| P176-Asn5 | XZ435 | g.Eco.SucC | g.Pgi.SucD | g.Pgi.4hbD | g.Pgi.Cat2.abfT-1 | g.Cac.adhe2 | g.Aba.ADP1 |
| P176-Asn6 | XZ342 | g.Eco.SucC | g.Pgi.SucD | g.Pgi.4hbD | g.Pgi.Cat2.abfT-2 | g.Cac.adhe2 | g.Aba.ADP1 |
| P176-Asn6 | XZ343 | g.Eco.SucC | g.Pgi.SucD | g.Pgi.4hbD | g.Pgi.Cat2.abfT-2 | g.Cac.adhe2 | g.Aba.ADP1 |
| P176-Asn6 | XZ436 | g.Eco.SucC | g.Pgi.SucD | g.Pgi.4hbD | g.Pgi.Cat2.abfT-2 | g.Cac.adhe2 | g.Aba.ADP1 |
| P176-Asn6 | XZ437 | g.Eco.SucC | g.Pgi.SucD | g.Pgi.4hbD | g.Pgi.Cat2.abfT-2 | g.Cac.adhe2 | g.Aba.ADP1 |
| P176-Asn7 | XZ344 | g.Eco.SucD | g.Pgi.SucD | g.Pgi.4hbD | g.Pgi.Cat2.abfT-1 | g.Cac.adhe2 | g.Aba.ADP1 |
| P176-Asn7 | XZ345 | g.Eco.SucD | g.Pgi.SucD | g.Pgi.4hbD | g.Pgi.Cat2.abfT-1 | g.Cac.adhe2 | g.Aba.ADP1 |
| P176-Asn7 | XZ438 | g.Eco.SucD | g.Pgi.SucD | g.Pgi.4hbD | g.Pgi.Cat2.abfT-1 | g.Cac.adhe2 | g.Aba.ADP1 |
| P176-Asn7 | XZ439 | g.Eco.SucD | g.Pgi.SucD | g.Pgi.4hbD | g.Pgi.Cat2.abfT-1 | g.Cac.adhe2 | g.Aba.ADP1 |
| P176-Asn8 | XZ346 | g.Eco.SucD | g.Pgi.SucD | g.Pgi.4hbD | g.Pgi.Cat2.abfT-2 | g.Cac.adhe2 | g.Aba.ADP1 |
| P176-Asn8 | XZ347 | g.Eco.SucD | g.Pgi.SucD | g.Pgi.4hbD | g.Pgi.Cat2.abfT-2 | g.Cac.adhe2 | g.Aba.ADP1 |
| P176-Asn8 | XZ440 | g.Eco.SucD | g.Pgi.SucD | g.Pgi.4hbD | g.Pgi.Cat2.abfT-2 | g.Cac.adhe2 | g.Aba.ADP1 |
| P176-Asn8 | XZ441 | g.Eco.SucD | g.Pgi.SucD | g.Pgi.4hbD | g.Pgi.Cat2.abfT-2 | g.Cac.adhe2 | g.Aba.ADP1 |
| P176-Asn9 | XZ442 | g.Eco.SucD | g.Pgi.SucD | g.Pgi.4hbD | g.Pgi.Cat2.abfT-1 | g.Eco.adhe | g.Eco.ahr |
| P176-Asn9 | XZ443 | g.Eco.SucD | g.Pgi.SucD | g.Pgi.4hbD | g.Pgi.Cat2.abfT-1 | g.Eco.adhe | g.Eco.ahr |
| P176-Asn10 | XZ444 | g.Eco.SucD | g.Pgi.SucD | g.Pgi.4hbD | g.Pgi.Cat2.abfT-2 | g.Eco.adhe | g.Eco.ahr |
| P176-Asn11 | XZ445 | g.Eco.SucD | g.Pgi.SucD | g.Eco.yihu | g.Pgi.Cat2.abfT-1 | g.Eco.adhe | g.Eco.ahr |
| P176-Asn12 | XZ446 | g.Eco.SucD | g.Pgi.SucD | g.Eco.yihu | g.Pgi.Cat2.abfT-2 | g.Eco.adhe | g.Eco.ahr |
| P176-Asn12 | XZ447 | g.Eco.SucD | g.Pgi.SucD | g.Eco.yihu | g.Pgi.Cat2.abfT-2 | g.Eco.adhe | g.Eco.ahr |
| P176-Asn15 | XZ448 | g.Eco.SucD | g.Ckl.SucD | g.Eco.yihu | g.Pgi.Cat2.abfT-1 | g.Eco.adhe | g.Eco.ahr |
| P176-Asn15 | XZ449 | g.Eco.SucD | g.Ckl.SucD | g.Eco.yihu | g.Pgi.Cat2.abfT-1 | g.Eco.adhe | g.Eco.ahr |
| P176-Asn18 | XZ348 | g.Eco.SucC | g.Pgi.SucD | g.Pgi.4hbD | g.Pgi.Cat2.abfT-2 | g.Cac.adhe2 | g.Aba.ADP1 |
| P176-Asn18 | XZ450 | g.Eco.SucC | g.Pgi.SucD | g.Pgi.4hbD | g.Pgi.Cat2.abfT-2 | g.Cac.adhe2 | g.Aba.ADP1 |
| P176-Asn18 | XZ451 | g.Eco.SucC | g.Pgi.SucD | g.Pgi.4hbD | g.Pgi.Cat2.abfT-2 | g.Cac.adhe2 | g.Aba.ADP1 |
| P176-Asn25 | XZ452 | g.Eco.SucC | g.Ckl.SucD | g.Eco.yihu | g.Pgi.Cat2.abfT-1 | g.Eco.adhe | g.Eco.ahr |
| P176-Asn25 | XZ453 | g.Eco.SucC | g.Ckl.SucD | g.Eco.yihu | g.Pgi.Cat2.abfT-1 | g.Eco.adhe | g.Eco.ahr |
| P176-Asn26 | XZ454 | g.Eco.SucC | g.Ckl.SucD | g.Eco.yihu | g.Pgi.Cat2.abfT-2 | g.Eco.adhe | g.Eco.ahr |
| P176-Asn34 | XZ460 | g.Eco.SucD | g.Pgi.SucD | g.Pgi.4hbD | g.Pgi.Cat2.abfT-2 | g.Eco.adhe | g.Eco.ahr |
| P176-Asn34 | XZ461 | g.Eco.SucD | g.Pgi.SucD | g.Pgi.4hbD | g.Pgi.Cat2.abfT-2 | g.Eco.adhe | g.Eco.ahr |
| P176-Asn35 | XZ462 | g.Eco.SucD | g.Pgi.SucD | g.Eco.yihu | g.Pgi.Cat2.abfT-1 | g.Eco.adhe | g.Eco.ahr |
| P176-Asn36 | XZ463 | g.Eco.SucD | g.Pgi.SucD | g.Eco.yihu | g.Pgi.Cat2.abfT-2 | g.Eco.adhe | g.Eco.ahr |
| P176-Asn36 | XZ464 | g.Eco.SucD | g.Pgi.SucD | g.Eco.yihu | g.Pgi.Cat2.abfT-2 | g.Eco.adhe | g.Eco.ahr |
| P176-Asn38 | XZ349 | g.Eco.SucC | g.Ckl.SucD | g.Eco.yihu | g.Pgi.Cat2.abfT-2 | g.Eco.adhe | g.Eco.ahr |
| P176-Asn38 | XZ350 | g.Eco.SucC | g.Ckl.SucD | g.Eco.yihu | g.Pgi.Cat2.abfT-2 | g.Eco.adhe | g.Eco.ahr |
| P176-Asn38 | XZ465 | g.Eco.SucC | g.Ckl.SucD | g.Eco.yihu | g.Pgi.Cat2.abfT-2 | g.Eco.adhe | g.Eco.ahr |
| P176-Asn38 | XZ466 | g.Eco.SucC | g.Ckl.SucD | g.Eco.yihu | g.Pgi.Cat2.abfT-2 | g.Eco.adhe | g.Eco.ahr |
| P176-Asn40 | XZ467 | g.Eco.SucD | g.Ckl.SucD | g.Eco.yihu | g.Pgi.Cat2.abfT-2 | g.Eco.adhe | g.Eco.ahr |
| P176-Asn40 | XZ468 | g.Eco.SucD | g.Ckl.SucD | g.Eco.yihu | g.Pgi.Cat2.abfT-2 | g.Eco.adhe | g.Eco.ahr |
| P176-Asn41 | XZ351 | g.Eco.SucC | g.Pgi.SucD | g.Pgi.4hbD | g.Pgi.Cat2.abfT-1 | g.Cac.adhe2 | g.Aba.ADP1 |
| P176-Asn41 | XZ352 | g.Eco.SucC | g.Pgi.SucD | g.Pgi.4hbD | g.Pgi.Cat2.abfT-1 | g.Cac.adhe2 | g.Aba.ADP1 |
| P176-Asn46 | XZ469 | g.Eco.SucD | g.Pgi.SucD | g.Pgi.4hbD | g.Pgi.Cat2.abfT-2 | g.Eco.adhe | g.Eco.ahr |
| P176-Asn46 | XZ470 | g.Eco.SucD | g.Pgi.SucD | g.Pgi.4hbD | g.Pgi.Cat2.abfT-2 | g.Eco.adhe | g.Eco.ahr |

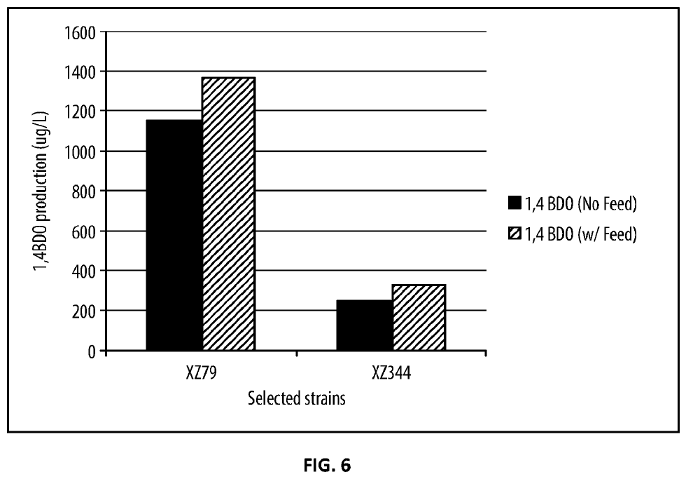

Several of the top engineered strains XZ79 (p.BAD>Mbo.SucA>Ckl.4hbD>Pgi.abft-1>Cac.adhe2>Aba.ADP1) XZ344 and (p.BAD>Eco.SucD>Pgi.SucD>Pgi.4hbD>Pgi.abft-1>Cac.adhe2>Aba.ADP1) were selected based on the results from small scale microtiter plate analysis. These strains were able to produce detectable 1,4 BDO without feeding intermediates α-ketoglutarate or succinic acid. To determine if XZ79 and XZ344 were able to produce detectable 1,4 BDO directly from methane in a shake bottle experiment, we tested XZ79 and XZ344 in media with and without supplementation of the intermediates. As shown in FIG. 6, XZ79 and XZ344 both produced detectable amount of 1,4 BDO directly from methane. Feeding α-ketoglutarate or succinic acid increased the 1,4 BDO productivity.

### Example 5:1,4-BDO Production Directly from Methane in Shake Bottles

We tested 35 and 15 different broad host plasmids containing variations in the 5-gene 1,4 BDO pathway, from the α-ketoglutarate and succinate pathway respectively, and transformed the aforementioned plasmids into a transformation competent methanotroph strain, RL83A, and evaluated the strains for 1,4 BDO production in shake bottles using methane as the carbon source. For these experiments, starter cultures were inoculated into 125 ml shake bottles containing 10 ml of media. The bottles were fed a source of methane and incubated in a shaker at 37° C. for 72 hours. After 72 h, the cultures were fed 1% of arabinose and α-ketoglutarate or succinate. Samples were collected after 96 hours and tested.

The genotypes and relevant production titers for each of the strains are provided in Table 7 (α-ketoglutarate pathway) and Table 8 (succinate pathway) below. For the fermentation of the strains utilizing the α-ketoglutarate pathway, the media contained in the bottles were supplemented with 400 mg/L of α-ketoglutarate. For the fermentation of the strains utilizing the succinate pathway, the media contained in the bottles were supplemented with 200 mg/L of succinate.

TABLE 7
Genotypes of Microorganisms tested for the α-ketoglutarate pathway

| | Productivity | | | | | | |
|---|---|---|---|---|---|---|---|
| | GHB | 1,4 BDO | Genotype | | | | |
| Strain | (μg/L) | (μg/L) | kgd | 4hbD | Cat2 | Ald | Adh |
| XZ75 | 211.724 | 69.536 | g.Mbo.SucA | g.Eco.yihu | g.Pgi.Cat2.abfT-1 | g.Cac.adhe2 | g.Aba.ADP1 |
| XZ76 | 208.769 | 118.376 | g.Mbo.SucA | g.Eco.yihu | g.Pgi.Cat2.abfT-1 | g.Cac.adhe2 | g.Aba.ADP1 |
| XZ77 | | | g.Mbo.SucA | g.Pgi.4hbD | g.Pgi.Cat2.abfT-1 | g.Cac.adhe2 | g.Aba.ADP1 |
| XZ78 | | | g.Mbo.SucA | g.Pgi.4hbD | g.Pgi.Cat2.abfT-1 | g.Cac.adhe2 | g.Aba.ADP1 |
| XZ79 | 875.47 | 260.966 | g.Mbo.SucA | g.Ckl.4hbD | g.Pgi.Cat2.abfT-1 | g.Cac.adhe2 | g.Aba.ADP1 |
| XZ80 | 562.381 | 174.142 | g.Mbo.SucA | g.Ckl.4hbD | g.Pgi.Cat2.abfT-1 | g.Cac.adhe2 | g.Aba.ADP1 |
| XZ81 | | | g.Mbo.SucA | g.Eco.yihu | g.Pgi.Cat2.abfT-2 | g.Cac.adhe2 | g.Aba.ADP1 |
| XZ82 | | | g.Mbo.SucA | g.Eco.yihu | g.Pgi.Cat2.abfT-2 | g.Cac.adhe2 | g.Aba.ADP1 |
| XZ83 | | | g.Mbo.SucA | g.Eco.yihu | g.Cam.abft | g.Cac.adhe2 | g.Aba.ADP1 |
| XZ84 | 461.119 | | g.Mbo.SucA | g.Eco.yihu | g.Cam.abft | g.Cac.adhe2 | g.Aba.ADP1 |
| XZ85 | | | g.Mbo.SucA | g.Eco.yihu | g.Pgi.Cat2.abfT-1 | g.Eco.yqhD | g.Aba.ADP1 |
| XZ86 | | | g.Mbo.SucA | g.Eco.yihu | g.Pgi.Cat2.abfT-1 | g.Eco.yqhD | g.Aba.ADP1 |
| XZ87 | | | g.Mbo.SucA | g.Eco.yihu | g.Pgi.Cat2.abfT-1 | g.Eco.fucO | g.Aba.ADP1 |
| XZ88 | 530.438 | | g.Mbo.SucA | g.Eco.yihu | g.Pgi.Cat2.abfT-1 | g.Eco.fucO | g.Aba.ADP1 |
| XZ89 | | | g.Mtu.kgd | g.Eco.yihu | g.Pgi.Cat2.abfT-1 | g.Cac.adhe2 | g.Aba.ADP1 |
| XZ90 | 109.323 | 30.634 | g.Cte.kgd | g.Eco.yihu | g.Pgi.Cat2.abfT-1 | g.Cac.adhe2 | g.Aba.ADP1 |
| XZ91 | | | g.Cte.kgd | g.Eco.yihu | g.Pgi.Cat2.abfT-1 | g.Cac.adhe2 | g.Aba.ADP1 |
| XZ92 | | | g.Rjo.odhA | g.Eco.yihu | g.Pgi.Cat2.abfT-1 | g.Cbe.Ald | g.Aba.ADP1 |
| XZ93 | | | g.Rjo.odhA | g.Eco.yihu | g.Pgi.Cat2.abfT-1 | g.Cbe.Ald | g.Aba.ADP1 |
| XZ94 | 410.306 | | g.Mbo.SucA | g.Eco.yihu | g.Pgi.Cat2.abfT-1 | g.Eco.yqhD | g.Sce.adh6 |
| XZ95 | 354.687 | | g.Mbo.SucA | g.Eco.yihu | g.Pgi.Cat2.abfT-1 | g.Eco.yqhD | g.Sce.adh6 |
| XZ96 | | | g.Mbo.SucA | g.Eco.yihu | g.Pgi.Cat2.abfT-1 | g.Eco.fucO | g.Sce.adh6 |
| XZ97 | | | g.Mbo.SucA | g.Eco.yihu | g.Pgi.Cat2.abfT-1 | g.Eco.adhe | g.Sce.adh6 |
| XZ98 | | | g.Mbo.SucA | g.Eco.yihu | g.Pgi.Cat2.abfT-1 | g.Eco.adhe | g.Sce.adh6 |
| XZ99 | | | g.Mbo.SucA | g.Eco.yihu | g.Pgi.Cat2.abfT-2 | g.Eco.adhe | g.Eco.ahr |
| XZ100 | | | g.Mbo.SucA | g.Eco.yihu | g.Pgi.Cat2.abfT-2 | g.Eco.adhe | g.Eco.ahr |
| XZ216 | 2067.802 | | g.Mbo.SucA | g.Pgi.4hbD | g.Pgi.Cat2.abfT-2 | g.Eco.adhe | g.Sce.adh6 |
| XZ217 | | | g.Mbo.SucA | g.Pgi.4hbD | g.Pgi.Cat2.abfT-2 | g.Eco.adhe | g.Sce.adh6 |
| XZ276 | 535.295 | | g.Mbo.SucA | g.Eco.yihu | g.Pgi.Cat2.abfT-1 | g.Eco.adhe | g.Aba.ADP1 |
| XZ277 | | | g.Mbo.SucA | g.Eco.yihu | g.Pgi.Cat2.abfT-1 | g.Eco.adhe | g.Aba.ADP1 |
| XZ278 | 163.658 | 52.054 | g.Mbo.SucA | g.Eco.yihu | g.Pgi.Cat2.abfT-1 | g.Cac.adhe2 | g.Sce.adh6 |
| XZ279 | | | g.Mbo.SucA | g.Eco.yihu | g.Pgi.Cat2.abfT-1 | g.Cac.adhe2 | g.Sce.adh6 |
| XZ280 | | | g.Mbo.SucA | g.Eco.yihu | g.Pgi.Cat2.abfT-1 | g.Cac.adhe2 | g.Eco.ahr |
| XZ281 | | | g.Mbo.SucA | g.Eco.yihu | g.Pgi.Cat2.abfT-1 | g.Cac.adhe2 | g.Eco.ahr |

| | |
|---|---|
| RL83 | Parental strain control |

TABLE 8
Genotypes of Microorganisms tested for the Succinate pathway

| | GHB | 1,4 BDO | | | | | | |
|---|---|---|---|---|---|---|---|---|
| Strain | (μg/l) | (μg/l) | Genotype | | | | | |
| XZ340 | 133.3 | | g.Eco.SucC | g.Pgi.SucD | g.Pgi.4hbD | g.Pgi.Cat2.abfT-1 | g.Cac.adhe2 | g.Aba.ADP1 |
| XZ341 | 101.9 | | g.Eco.SucC | g.Pgi.SucD | g.Pgi.4hbD | g.Pgi.Cat2.abfT-1 | g.Cac.adhe2 | g.Aba.ADP1 |
| XZ343 | 1711.4 | 31.6 | g.Eco.SucC | g.Pgi.SucD | g.Pgi.4hbD | g.Pgi.Cat2.abfT-2 | g.Cac.adhe2 | g.Aba.ADP1 |
| XZ344 | 1789.8 | 344.4 | g.Eco.SucD | g.Pgi.SucD | g.Pgi.4hbD | g.Pgi.Cat2.abfT-1 | g.Cac.adhe2 | g.Aba.ADP1 |
| XZ345 | 1926.9 | 579.4 | g.Eco.SucD | g.Pgi.SucD | g.Pgi.4hbD | g.Pgi.Cat2.abfT-1 | g.Cac.adhe2 | g.Aba.ADP1 |
| XZ346 | 5512.5 | 268.2 | g.Eco.SucD | g.Pgi.SucD | g.Pgi.4hbD | g.Pgi.Cat2.abfT-2 | g.Cac.adhe2 | g.Aba.ADP1 |
| XZ347 | 5131.2 | 244.6 | g.Eco.SucD | g.Pgi.SucD | g.Pgi.4hbD | g.Pgi.Cat2.abfT-2 | g.Cac.adhe2 | g.Aba.ADP1 |
| XZ434 | 112.3 | | g.Eco.SucC | g.Pgi.SucD | g.Pgi.4hbD | g.Pgi.Cat2.abfT-1 | g.Cac.adhe2 | g.Aba.ADP1 |
| XZ435 | 165.2 | | g.Eco.SucC | g.Pgi.SucD | g.Pgi.4hbD | g.Pgi.Cat2.abfT-1 | g.Cac.adhe2 | g.Aba.ADP1 |
| XZ436 | 3983.5 | 225.3 | g.Eco.SucC | g.Pgi.SucD | g.Pgi.4hbD | g.Pgi.Cat2.abfT-2 | g.Cac.adhe2 | g.Aba.ADP1 |
| XZ437 | 1362.9 | 106.5 | g.Eco.SucC | g.Pgi.SucD | g.Pgi.4hbD | g.Pgi.Cat2.abfT-2 | g.Cac.adhe2 | g.Aba.ADP1 |
| XZ438 | 3200.1 | 524.8 | g.Eco.SucD | g.Pgi.SucD | g.Pgi.4hbD | g.Pgi.Cat2.abfT-1 | g.Cac.adhe2 | g.Aba.ADP1 |
| XZ439 | 3387.2 | 313 | g.Eco.SucD | g.Pgi.SucD | g.Pgi.4hbD | g.Pgi.Cat2.abfT-1 | g.Cac.adhe2 | g.Aba.ADP1 |
| XZ440 | 7580.2 | 365.8 | g.Eco.SucD | g.Pgi.SucD | g.Pgi.4hbD | g.Pgi.Cat2.abfT-2 | g.Cac.adhe2 | g.Aba.ADP1 |
| XZ441 | 6103.7 | 245.2 | g.Eco.SucD | g.Pgi.SucD | g.Pgi.4hbD | g.Pgi.Cat2.abfT-2 | g.Cac.adhe2 | g.Aba.ADP1 |

### Example 6: Increased Succinate Production

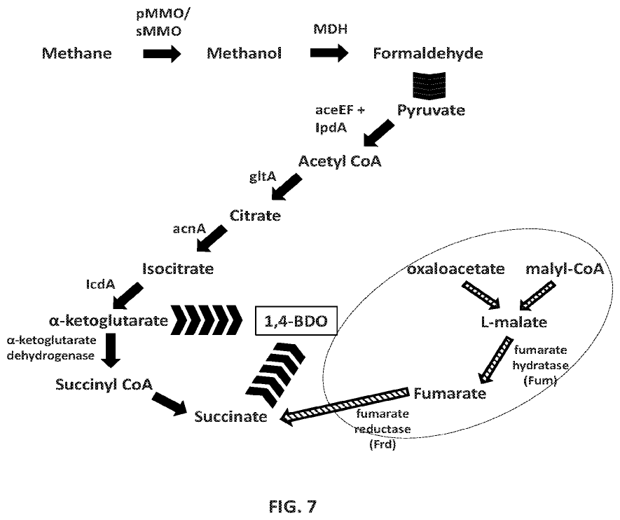

In order to increase 1,4-BDO titers, the production of the upstream product succinate was measured in the presence or absence of fumarate reductase (frd) genes. See FIG. 7. 58 Methylococcus capsulatus methanotroph strains were generated comprising E. coli FrdA, E. coli FrdB, E. coli FrdC, E. coli FrdD with varying promoters. Each strain was inoculated into 125 ml shake bottles containing 10 ml of media to create a start culture. The starter cultures were then used to inoculate a shake bottle at an initial OD of 0.1. The shake bottles were incubated for 96 hours at 37° C. in the presence of excess methane. Succinate titers were then measured.

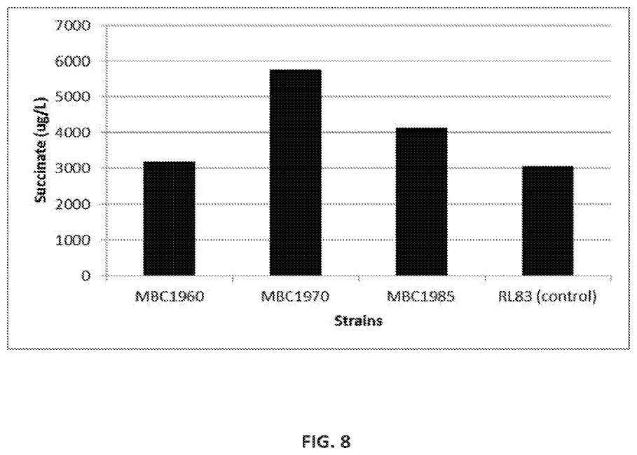

All the strains MBC1960, MBC1970, and MBC1985 expressed a heterologous E. coli FrdA, FrdB, FrdC, and FrdD but were under the control of different promoters (pmxaF, J23111, and J23100, respectively). See FIG. 8. As seen in FIG. 8, the MBC1970 and MBC1985 strains produced significantly more succinate than its parent control strain (RL83).

1-141. (canceled)

142. A method of making succinate comprising:
(a) contacting a C1 carbon with a genetically-modified microorganism that comprises a heterologous fumarate hydratase and/or a heterologous fumarate reductase; and
(b) growing the genetically microorganism to produce succinate.

143. The method of claim 142, wherein the genetically microorganism comprises a pyruvate dehydrogenase (aceEF), citrate synthase (gltA), aconitate hydratase 1 (acnA), and/or isocitrate dehydrogenase (icdA).

144. The method of claim 142, wherein the succinate from (b) is isolated.

145. The method of claim 142, wherein the succinate is converted into 1,4-BDO.

146. The method of claim 142, wherein the genetically-modified microorganism is grown for at least 96 hours.

147. The method of claim 142, wherein the fumarate hydratase comprises an amino acid sequence having at least 90% identity with any one of SEQ ID NOs: 45, 47, and 49.

148. The method of claim 142, wherein the fumarate hydratase comprises an amino acid sequence having at least 95% identity with any one of SEQ ID NOs: 45, 47, and 49.

149. The method of claim 142, wherein the fumarate hydratase comprises an amino acid sequence having at least 98% identity with any one of SEQ ID NOs: 45, 47, and 49.

150. The method of claim 142, wherein the fumarate hydratase comprises the amino acid sequence of any one of SEQ ID NOs: 45, 47, and 49.

151. The method of claim 142, wherein the fumarate reductase is from a bacteria.

152. The method of claim 142, wherein the fumarate reductase comprises an amino acid sequence having at least 90% identity with any one of SEQ ID NOs: 51, 53, 55, and 57.

153. The method of claim 142, wherein the fumarate reductase comprises an amino acid sequence having at least 95% identity with any one of SEQ ID NOs: 51, 53, 55, and 57.

154. The method of claim 142, wherein the fumarate reductase comprises an amino acid sequence having at least 98% identity with any one of SEQ ID NOs: 51, 53, 55, and 57.

155. The method of claim 142, wherein the fumarate reductase comprises the amino acid sequence of any one of SEQ ID NOs: 51, 53, 55, and 57.

156. The method of claim 142, wherein the genetically-modified microorganism is a methanotroph.

157. The method of claim 142, wherein the genetically-modified microorganism is from the genus Methylococcus.

158. The method of claim 142, wherein the genetically modified microorganism is an M. capsulatus.

159. The method of claim 142, wherein the genetically-modified microorganism comprises a heterologous fumarate reductase gene under the control of a pMxaF, J23111, or J23100 promoter.

160. The method of claim 142, wherein the genetically-modified microorganism comprises a heterologous fumarate reductase gene under the control of a J23111 or J23100 promoter.

161. The method of claim 142, wherein the genetically modified microorganism comprises a heterologous fumarate reductase gene under the control of a J23111 promoter.


SEQUENCE LISTING (58 sequences)

SEQ ID NO: 1 - protein, 1231 aa, Mycobacterium bovis
MANISSPFGQNEWLVEAMYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYMYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 2 - DNA, 3696 bp, Mycobacterium bovis
  source 1..3696: mol_type = genomic DNA
atggccaatatctcgtccccgttcggccagaacgagtggctcgtggaggccatgtatcggaagttccgggacgacccgtccagcgtcgacccgtcctggcacgagttcctcgtggactactcccccgagccgaccagccagccggcggccgagccgacccgggtcacgtcgccgctcgtcgccgaacgggccgccgccgccgccccccaggcgccccccaagccggccgacacggccgccgccggcaacggcgtggtggcggccctggccgccaagaccgccgtgccgcccccggccgaaggcgacgaagtcgccgtcctccggggcgcggcggcggcggtcgtgaagaacatgagcgcgtccctggaggtgccgaccgccacgtcggtccgggccgtccccgcgaaactcctcatcgacaaccgcatcgtcatcaataaccagctgaagcggacgcggggcggcaagatctcgttcacgcacctcctcggctacgcgctcgtgcaggccgtcaagaagttcccgaacatgaaccggcactacacggaagtggacggcaaacccacggccgtcacccccgcccatacgaacctcggcctggccatcgacctgcagggcaaggacggcaagcggtcgctggtggtcgccggcatcaagcgctgcgagacgatgcgcttcgcgcagttcgtcacggcctatgaagacatcgtccgccgggcccgggacggcaagctcaccacggaagatttcgcgggcgtcaccatctcgctcaccaatcccggcacgatcggcaccgtccatagcgtgccgcgcctgatgcccggccagggcgcgatcatcggcgtcggcgcgatggaataccccgccgagttccagggcgcgtccgaggagcggatcgcggaactgggcatcggcaagctcatcaccctcaccagcacgtatgaccaccgcatcatccagggcgccgagtcgggcgacttcctccggacgatccatgagctgctgctgagcgacggcttctgggatgaggtgttccgggaactgtccatcccgtatctgccggtccggtggagcacggacaaccccgatagcatcgtggacaaaaacgcccgcgtgatgaacctcatcgccgcctaccgcaaccgcggccacctgatggccgacaccgatccgctgcggctcgacaaagcccgcttccggagccatcccgacctggaagtcctgacccacggcctcaccctgtgggatctggatcgggtcttcaaggtcgacggcttcgccggcgcccagtacaagaagctgcgcgatgtgctcggcctgctgcgggacgcctattgccgccacatcggcgtggagtacgcgcacatcctcgacccggaacagaaagaatggctggaacagcgcgtcgaaaccaagcatgtgaagcccacggtggcgcagcagaagtacatcctgtcgaagctgaatgcggccgaagccttcgaaaccttcctgcagaccaagtacgtcggccagaagcgcttctccctggaaggcgccgagtccgtcatcccgatgatggatgccgcgatcgaccagtgcgccgaacacggcctggacgaagtcgtgatcggcatgccccatcgcggccggctcaacgtcctcgcgaacatcgtcggcaagccgtactcgcagatcttcacggagttcgaaggcaacctgaacccctcccaggcccacggcagcggcgacgtgaaatatcacctgggcgccaccggcctgtatctgcagatgttcggcgacaacgacatccaggtcagcctgaccgccaacccctcgcatctggaagccgtcgaccccgtcctggaaggcctggtccgcgcgaagcaggacctcctggaccacggcagcatcgatagcgacggccagcgggccttctccgtggtgcccctcatgctgcacggcgacgccgccttcgccggccagggcgtggtcgccgagacgctcaacctggcgaacctgcccggctaccgcgtcggcggcacgatccacatcatcgtcaacaatcagatcggcttcaccaccgcccccgagtactcccgcagctcggaatactgcaccgatgtggcgaaaatgatcggcgccccgatcttccatgtcaatggcgatgatccggaagcgtgcgtgtgggtggcccgcctggcggtggatttccgccagcggttcaagaaggatgtggtgatcgacatgctgtgctaccgccgccggggccataatgaaggcgacgatccctccatgaccaacccctacatgtacgatgtcgtggacacgaagcgcggcgcccgcaagtcctataccgaggccctcatcggccggggcgatatcagcatgaaagaagccgaggatgcgctccgcgactatcagggccagctggaacgggtgttcaacgaggtgcgggaactcgaaaaacacggcgtccagccgtccgagagcgtggagtccgatcagatgatcccggcgggcctcgccacggcggtcgataagtccctgctggcccggatcggcgatgccttcctggccctccccaatggcttcacggcgcacccgcgggtgcagccggtgctcgaaaaacgccgggaaatggcctacgaaggcaagatcgactgggccttcggcgagctgctggcgctgggctccctggtggccgaaggcaaactggtgcgcctgtccggccaggactcgcgccgcggcaccttctcccagcgccactcggtcctgatcgatcgccataccggcgaggagttcaccccgctccagctcctggccacgaactccgacggctcgcccaccggcggcaagttcctggtgtatgacagccccctgagcgagtatgcggcggtcggcttcgagtacggctacaccgtcggcaatcccgacgccgtcgtcctgtgggaagcgcagttcggcgacttcgtcaatggcgcccagtcgatcatcgacgagttcatctcgtcgggcgaggccaaatggggccagctctcgaacgtcgtgctgctgctcccgcacggccacgaaggccagggcccggatcacacctccgcccggatcgaacggttcctgcagctctgggccgagggctccatgacgatcgccatgcccagcaccccgtccaactatttccacctcctgcggcgccatgcgctcgacggcatccagcggcccctgatcgtcttcacgccgaagagcatgctgcgccataaggccgccgtctcggagatcaaggacttcaccgagatcaaattccggtcggtgctggaagaaccgacctatgaagatggcatcggcgaccgcaataaggtgtcgcgcatcctgctgacctcgggcaagctgtactacgagctggccgcccgcaaggccaaagacaaccggaacgacctggccatcgtccggctggagcagctggcgcccctcccgcgccggcgcctgcgcgaaaccctggatcgctatgagaacgtcaaggagttcttctgggtgcaggaggaacccgcgaatcagggcgcctggccccgcttcggcctggagctgccggagctgctccccgacaagctggccggcatcaagcggatcagccgccgcgcgatgtcggccccgagctcgggctccagcaaggtccatgcggtcgagcagcaggagatcctggatgaagcgttcggctga

SEQ ID NO: 3 - protein, 1231 aa, Mycobacterium tuberculosis
MANISSPFGQNEWLVEEMYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYVYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 4 - DNA, 3696 bp, Mycobacterium tuberculosis
  source 1..3696: mol_type = genomic DNA
atggccaacatcagcagccccttcggccagaatgaatggctcgtggaggagatgtaccggaagttccgggatgatcccagctcggtcgatccgtcgtggcacgagttcctggtcgactatagccccgaacccacctcccagccggcggccgagcccacgcgggtgacctccccgctggtcgcggaacgcgccgcggcggcggccccccaggccccgccgaaacccgccgatacggccgccgcgggcaacggcgtcgtcgcggccctggccgcgaagaccgccgtgcccccccccgccgagggcgacgaggtggcggtgctccggggcgccgccgcggccgtggtcaagaacatgagcgcctccctcgaagtgccgaccgccaccagcgtccgggcggtgccggccaagctgctgatcgataatcggatcgtcatcaacaatcagctgaaacggacccgcggcggcaagatcagcttcacccatctgctcggctatgcgctggtccaggccgtgaagaagttccccaacatgaatcgccactataccgaagtcgacggcaaacccaccgccgtgaccccggcgcacacgaacctcggcctggcgatcgacctgcagggcaaggacggcaagcggagcctcgtggtggccggcatcaaacggtgcgagacgatgcggttcgcgcagttcgtgacggcctatgaggatatcgtgcgccgcgcccgggacggcaaactgaccaccgaagacttcgccggcgtcacgatctcgctgaccaacccgggcaccatcggcacggtccattcggtcccgcgcctcatgccgggccagggcgccatcatcggcgtgggcgcgatggaatatcccgccgagttccagggcgcctccgaggagcggatcgccgagctgggcatcggcaaactcatcacgctgacctccacgtatgaccaccgcatcatccagggcgcggaaagcggcgacttcctccggacgatccatgagctgctgctctccgacggcttctgggatgaagtgttccgcgagctgtcgatcccctatctcccggtgcgctggagcaccgacaacccggactcgatcgtggacaaaaacgcccgcgtcatgaatctgatcgccgcgtaccgcaatcggggccacctcatggcggacacggaccccctgcggctcgataaggcccggttccgctcccaccccgacctcgaagtcctgacccacggcctgaccctgtgggacctggatcgcgtcttcaaggtcgatggcttcgccggcgcccagtataagaagctgcgcgatgtgctgggcctgctccgcgatgcctattgccggcatatcggcgtggagtacgcccacatcctggaccccgagcagaaagaatggctggaacagcgcgtcgaaacgaagcacgtcaagcccaccgtcgcgcagcagaaatacatcctcagcaaactcaacgccgccgaagcgttcgaaacgttcctccagacgaagtacgtgggccagaaacgcttcagcctggaaggcgcggagagcgtcatccccatgatggacgccgccatcgaccagtgcgccgaacacggcctggatgaagtggtcatcggcatgccgcaccgcggccggctcaacgtcctggccaatatcgtgggcaagccctacagccagatcttcaccgagttcgaaggcaacctgaacccgtcgcaggcgcacggctcgggcgatgtcaagtaccatctcggcgccacgggcctgtatctgcagatgttcggcgacaacgacatccaggtgtccctgacggcgaaccccagccacctggaggcggtcgatcccgtcctggaaggcctggtgcgggccaagcaggatctcctcgaccacggctcgatcgactccgatggccagcgggcgttcagcgtggtgcccctcatgctgcatggcgacgcggcgttcgcgggccagggcgtggtggcggaaaccctcaacctcgcgaacctcccgggctatcgggtgggcggcaccatccatatcatcgtgaacaaccagatcggcttcaccacggcgccggaatattcccggagctcggaatattgcacggacgtggccaagatgatcggcgccccgatcttccacgtcaatggcgatgacccggaggcctgtgtgtgggtcgcccggctggccgtcgatttccgccagcgcttcaaaaaagatgtggtcatcgacatgctctgctaccgccgccggggccataatgagggcgacgacccctccatgacgaacccgtacgtctacgacgtggtcgacaccaagcgcggcgcccgcaagtcctatacggaggcgctcatcggccgcggcgacatctcgatgaaggaagcggaagacgcgctccgcgactaccagggccagctggagcgcgtcttcaacgaagtgcgggaactcgaaaagcatggcgtgcagccctccgaatcggtggagagcgatcagatgatcccggccggcctggccaccgccgtcgataaaagcctgctcgcccgcatcggcgacgccttcctggccctgccgaacggcttcacggcccatccccgcgtccagccggtgctggagaaacgccgcgaaatggcctacgagggcaagatcgattgggccttcggcgagctgctggcgctcggcagcctggtggccgaaggcaagctcgtgcggctctccggccaggactcgcggcgcggcacgttctcgcagcgccattccgtgctgatcgaccggcacaccggcgaagagttcacccccctccagctgctggccaccaattcggacggcagcccgacgggcggcaagttcctggtctatgactccccgctgagcgagtatgccgccgtcggcttcgagtatggctacaccgtcggcaatccggacgcggtcgtgctgtgggaagcgcagttcggcgacttcgtgaacggcgcccagtcgatcatcgatgagttcatctcgtcgggcgaagccaagtggggccagctcagcaacgtggtgctcctgctcccgcacggccacgagggccagggcccggatcatacgagcgcccgcatcgagcggttcctgcagctgtgggccgagggctccatgacgatcgcgatgccctcgacgccgtccaactacttccacctgctgcgccggcacgcgctggacggcatccagcgcccgctgatcgtcttcacccccaagagcatgctccggcacaaagccgccgtgtccgagatcaaggacttcacggagatcaaattccggagcgtgctggaggagcccacgtacgaggacggcatcggcgaccgcaacaaggtgtcccgcatcctcctgacgagcggcaagctgtactacgagctcgccgcccgcaaggccaaggataaccggaacgacctggccatcgtgcgcctcgaacagctggccccgctgccgcgccgccggctgcgggaaaccctggaccgctacgagaacgtcaaagagttcttctgggtccaggaagaaccggccaaccagggcgcgtggccgcgcttcggcctggaactgcccgagctcctgccggacaagctggccggcatcaagcgcatctcgcggcgggcgatgagcgccccgtcgagcggctcctccaaggtgcatgccgtggagcagcaggaaatcctggacgaggcgttcggctga

SEQ ID NO: 5 - protein, 1245 aa, Corynebacterium terpenotabidum
MNSTNFGSNGWLVDQMHQQFKEDPQSVDKEWRDFFTAGSASGPDAPETTAPTTTAATTTTATTSAAPATAQTSGVAVPSTAGTTHRASAAATVASNVALSANPIPERVAPPAPKDAAEPLTPGEAALKGAQRAIAKNMDASLDIPTATTVRDMPAKLMFENRAMINNHLRSQGRGKISFTHILGWAMVNAVKAHPTMNNNYKVIDGKPSVVTPEHINLGLAIDLVSKNGSRNLVVAAIRACETFDFEGFVDAYEDIVVRARKGKLTMDDFSGVTIQLTNPGGIGTRHSVPRLTHGQGAIIGVGAMDYPAEFAGASEDRLADLGVGKLVTITSTYDHRIIQGAESGEFLRDMSRQLIDDGFWDGIYASLKVPYAPVRWSQDVPNTGVDKSTRVMQLIEAYRSRGHLIADIDPLHWTQPGLPVPDHSDLDIESHGLTLWDFDRRFHVGGFAGRESMTLREVLATLRKAYTLKVGSEYTHILDKDERLWLQEHIEAGQQKLSNPEQKYLLQTLNSAEAFENFLQTKYIGQKRFSLEGAEALIPLLDAAADQAAEQGLEEVVIGMPHRGRLNVLANIVGKPYSTIFGEFEGNIEPAAAGGSGDVKYHLGAEGVYTQMFGDNDIKVTLTANPSHLEAVNPVMEGLARAHQDISPRAEDRPIMPILMHGDAAFTGLGIVPETINMAQLRGYSVGGTVHVVVNNQIGFTTTPDRGRSTHYATDIAKGFDCPVFHVNGDDPEAVVWVARLAVEYRRRFGKDVFIDLVCYRRRGHNEADDPSMTQPELYSIIESRPTVRSLYHDTLVGRGDITAEDAQRAADDFHGQLESVFNQVKEGVKGVTPAAQTGIAGGQDLSTGLDTSITADVIAEIGDSYTVDAPEDFNVHQRVKPVVKRRHQMSRQGKIDWAFGELLAFGSLAREGRLVRLAGEDSQRGTFTQRHAILFDSTTNKPFSPLEMVARNSGNGGSFRAFNSPLTEYAGMGFEYGYSVGNLDAVVAWEAQFGDFADGAQTIIDEYISSGEAKWGQLSSVILLLPHGYEGQGPDHSSARIERYLQMAAEGSMTIAQPSTPANHFHLLRRHALGTMRRPLVVFTPKSMLRNKAAVSSVEDFTEVTKFRSVLDDPRFADGTADRSGVKTVLMCSGKIYYDLEKKRAEDGRDDIAIVRVEMLHPIPHNRIRETVVEGYPGAEVRWVQDEPANQGAWPFLALNLPERIPGFTMKRVSRRAQSSTATGVAKVHHLEQETLLTEAFAT

SEQ ID NO: 6 - DNA, 3738 bp, Corynebacterium terpenotabidum
  source 1..3738: mol_type = genomic DNA
atgaactcgaccaacttcggcagcaatggctggctggtcgaccagatgcaccagcagttcaaggaagatccgcagtcggtcgacaaggaatggcgcgacttcttcaccgccggctcggcgtccggcccggatgccccggaaacgaccgccccgacgaccaccgccgccacgaccacgaccgcgaccacgagcgcggcccccgccaccgcgcagaccagcggcgtggccgtcccgtccaccgcgggcaccacccaccgggcctcggccgccgcgaccgtggcctcgaacgtggccctgagcgcgaacccgatccccgaacgggtcgcccccccggccccgaaggacgcggccgagccgctgaccccgggcgaggccgccctgaaaggcgcccagcgggcgatcgccaaaaacatggacgccagcctggatatccccaccgcgacgacggtccgcgacatgccggccaaactcatgttcgaaaaccgggccatgatcaacaaccatctgcgcagccagggccggggcaagatctcgttcacccacatcctgggctgggcgatggtcaacgccgtgaaagcccaccccaccatgaataacaattacaaggtcatcgacggcaagccgtcggtcgtgacccccgaacatatcaatctcggcctcgccatcgatctggtcagcaaaaacggctcgcggaacctggtcgtggccgccatccgcgcgtgcgaaaccttcgacttcgagggcttcgtggacgcctacgaggacatcgtggtccgcgcccggaagggcaaactgacgatggacgacttcagcggcgtcaccatccagctgaccaacccgggcggcatcggcacccgccactccgtcccgcgcctgacgcacggccagggcgcgatcatcggcgtgggcgcgatggactacccggcggagttcgccggcgcctcggaagaccgcctcgcggacctgggcgtgggcaaactggtgaccatcacgagcacgtatgaccaccggatcatccagggcgccgagtcgggcgagttcctgcgcgacatgagccgccagctcatcgacgatggcttctgggacggcatctacgccagcctgaaagtcccgtatgcgcccgtgcggtggtcgcaggacgtgcccaacaccggcgtcgacaagagcacccgcgtcatgcagctcatcgaggcgtaccgctcccgcggccacctgatcgccgacatcgacccgctgcattggacccagcccggcctgccggtgccggaccatagcgacctggacatcgaatcccacggcctgaccctgtgggacttcgaccggcgcttccacgtcggcggcttcgccggccgcgaatcgatgaccctgcgggaagtgctggccacgctgcggaaggcgtatacgctgaaggtcggctccgaatatacccatatcctcgacaaggacgaacgcctctggctccaggagcacatcgaggccggccagcagaagctcagcaaccccgaacagaagtatctgctgcagacgctgaactccgccgaggcgttcgaaaacttcctgcagaccaaatatatcggccagaagcgcttcagcctggaaggcgccgaagccctgatcccgctgctcgatgccgccgcggatcaggccgccgaacagggcctcgaagaggtggtgatcggcatgccgcatcgcggccggctgaatgtcctcgcgaacatcgtcggcaaaccgtacagcaccatcttcggcgagttcgagggcaacatcgagccggccgccgcgggcggctccggcgatgtcaaataccatctgggcgccgagggcgtgtatacgcagatgttcggcgacaacgacatcaaggtgacgctgaccgccaacccctcccacctggaagcggtcaatcccgtcatggagggcctcgcgcgggcgcatcaggatatctcgccccgggcggaggatcggccgatcatgccgatcctgatgcacggcgacgccgccttcaccggcctgggcatcgtcccggaaacgatcaacatggcccagctgcggggctactccgtgggcggcacggtgcatgtggtggtcaacaaccagatcggcttcaccacgacgccggatcgcggccggtccacgcactacgcgacggacatcgcgaagggcttcgattgccccgtcttccacgtcaacggcgacgatccggaggccgtcgtctgggtcgcccggctcgccgtggaataccggcgccgcttcggcaaagacgtgttcatcgacctggtgtgttatcggcgccgcggccataatgaagccgatgaccccagcatgacccagcccgaactgtacagcatcatcgagagccggccgacggtccggagcctgtatcatgacacgctggtcggccggggcgacatcacggcggaagacgcccagcgggccgccgacgacttccacggccagctggaaagcgtcttcaaccaggtcaaggaaggcgtgaagggcgtgaccccggcggcccagaccggcatcgccggcggccaggacctgtccaccggcctcgacaccagcatcaccgcggatgtgatcgccgagatcggcgattcctataccgtggatgcccccgaagacttcaatgtccaccagcgcgtgaagcccgtggtcaaacggcggcaccagatgtcgcgccagggcaagatcgattgggcgttcggcgaactcctcgcgttcggctcgctggcccgggaaggccggctggtccggctcgccggcgaggactcgcagcggggcaccttcacccagcggcacgccatcctgttcgacagcaccaccaacaagcccttcagccccctggaaatggtcgcgcggaactccggcaacggcggctcgttccgggccttcaattccccgctgacggaatatgcgggcatgggcttcgagtatggctactcggtgggcaacctggatgccgtggtggcctgggaggcgcagttcggcgacttcgcggacggcgcccagacgatcatcgacgagtacatctcctccggcgaggccaaatggggccagctgtcgagcgtgatcctgctgctcccgcacggctacgagggccagggcccggaccactcctccgcccgcatcgaacgctacctgcagatggccgccgaaggctcgatgaccatcgcgcagccgtcgacgcccgccaaccacttccacctgctccgccgccacgccctgggcaccatgcggcgcccgctggtcgtcttcaccccgaagtccatgctgcgcaacaaggcggccgtctcctcggtcgaagacttcacggaagtcaccaagttccggagcgtcctggatgacccccggttcgcggatggcaccgccgaccggagcggcgtcaagacggtgctgatgtgttccggcaagatctactatgatctggagaagaagcgggcggaagacggccgcgacgatatcgccatcgtccgcgtcgaaatgctgcatcccatcccgcataaccgcatccgcgaaacggtcgtggagggctaccccggcgcggaggtgcgctgggtgcaggacgagcccgccaaccagggcgcctggccgttcctggccctcaacctccccgagcgcatccccggcttcaccatgaagcgcgtcagccgccgggcccagagcagcaccgccacgggcgtggccaaggtccaccatctggagcaggagacgctgctgaccgaagccttcgccacctga

SEQ ID NO: 7 - protein, 1258 aa, Rhodococcus jostii
MSSSSTSQFGQNQWLVDEMYQRFQDDPSSVDASWHEFLTDYSPDAAAKAGAANGHGTNGTTTAAPAAAPSAKAATPPVPESETAPKPQTKTANGAAPKAAPNGAAPKAAAPKTEAPKKAAPAKETAATDAKASAPAPAVEESKVLRGAAAAVAKNMSASLAIPTATSVRAIPAKLMFDNRIVINNHLARTRGGKISFTHLLGYAIVQAVKAFPNMNRHFAEIDGKPNAVTPAHTNLGLAIDLPGKDGSRSLVVAAIKNTDTHNFTQFYSAYEDIVRRARDGKLTAEDFSGVTISLTNPGGIGTVHSVPRLMNGQGAIIGAGAMEYPAEFQGASDERLAEIGVGKLMTLTSTYDHRIIQGAESGDFLRTIHNLLISDEFYDEIFHALHIPYEPVRWRKDVPEGAVDKNTRVLELIAAYRNRGHLMADTDPLQFVKDKFRSHPDLDVRTHDLTLWDLDREFKVGGFHGQEKMKLRDVLSVLRDAYCRHVGVEYTHILEPEQQQWLQDRVEAHHVKPTVAQQKYILSKLNAAEAFETFLQTKYVGQKRFSLEGAESVIPMMDAVIDQAAEHQLDEVVIGMPHRGRLNVLANIVGKPYSKIFTEFEGNMNPAAAHGSGDVKYHLGAEGTYIQMFGDNDITVSLTANPSHLEAVDPVLEGLVRAKQDILDKGEDGFTVLPLMLHGDAAFAGQGVVAETLNLALLRGYRTGGTVHIVVNNQVGFTTAPEYSRSSEYCTDVAKMIGAPIFHVNGDDPEACVWVAQLAVDFREKFQKDVVIDMICYRRRGHNEGDDPSMTQPAMYDVIDTKRSVRKSYTESLIGRGDISLKEAEDALRDYQGQLERVFNEVRELEKYTPEPSESVELDQVLPTKLKTSVDESVLERIGDAFVNVPEGFTVHPRVKPVIEKRREMSREGKIDWAFAELLAFGSLVDQGKMVRLSGQDSKRGTFTQRHSVLIDRKTGAEYTPLQNLGSENPGKFLVYDSALSEFAAVGFEYGYSVGNPDALVLWEAQFGDFVNGAQSIIDEFISSGEAKWGQLSDVVLLLPHGHEGQGPDHTSGRIERFLQLCAEGSMTVAVPSTPASYFHLLRRHSLDGIRRPLVVFTPKSMLRNKAAVSDVEDFTTGKFRSVFEEPTYETGDAERDKVRRVLLVSGKLYWELLAKKQKDNREDIAIVRIEQLYPVPSRRLRETLDRYPNATEFRWVQEEPANQGAWPFFGLALPELLPDKLSGIKRISRRSMSAPSSGSSKVHAVEQQEIIDEAFG

SEQ ID NO: 8 - DNA, 3777 bp, Rhodococcus jostii
  source 1..3777: mol_type = genomic DNA
atgagctcgtcctcgacctcccagttcggccagaaccagtggctcgtggatgaaatgtaccagcgcttccaggatgatccgtcgtccgtggacgcctcctggcatgagttcctgacggactatagccccgacgccgccgccaaggcgggcgcggcgaacggccacggcaccaacggcaccaccacggcggcgccggcggcggcgccgtccgccaaagccgcgacccccccggtccccgagagcgagacggccccgaaaccccagaccaagacggcgaacggcgcggcgccgaaagccgcgcccaacggcgcggcgccgaaggccgcggcccccaagaccgaggcccccaagaaggccgcccccgcgaaggaaacggccgcgacggacgcgaaagcctcggccccggcccccgccgtggaggaatccaaggtgctgcggggcgccgccgccgccgtcgccaagaatatgtccgcgtcgctcgccatcccgaccgccacctccgtccgggccatcccggcgaagctgatgttcgacaaccgcatcgtcatcaacaaccacctggcgcggacccggggcggcaagatctcgttcacccatctgctgggctacgcgatcgtgcaggccgtgaaagcgttccccaacatgaaccgccacttcgcggagatcgatggcaagcccaatgcggtgacccccgcccataccaatctgggcctcgcgatcgatctgccgggcaaagatggcagccgctcgctggtcgtcgcggccatcaagaacaccgatacgcacaatttcacccagttctatagcgcctacgaggatatcgtgcgccgggcgcgggacggcaaactgaccgccgaagacttcagcggcgtcacgatctcgctgaccaacccgggcggcatcggcaccgtccattccgtcccgcgcctgatgaacggccagggcgccatcatcggcgcgggcgcgatggagtacccggccgagttccagggcgcctcggacgagcggctggccgaaatcggcgtcggcaagctgatgaccctcacgagcacctacgatcatcggatcatccagggcgccgaatccggcgacttcctccggacgatccataatctgctcatcagcgacgagttctacgacgaaatcttccatgccctgcatatcccctatgaacccgtccggtggcgcaaagacgtgcccgaaggcgccgtggataagaacacccgggtcctggagctcatcgccgcctaccggaaccgcggccacctgatggcggacaccgacccgctgcagttcgtgaaggacaagttccggtcgcacccggacctcgacgtgcgcacccatgatctgacgctctgggacctggatcgggagttcaaagtgggcggcttccacggccaggagaagatgaagctgcgcgatgtcctgtcggtgctgcgcgatgcgtattgtcgccatgtcggcgtggagtatacccacatcctggagccggagcagcagcagtggctgcaggatcgcgtcgaggcccaccacgtcaaaccgacggtcgcccagcagaagtatatcctgtccaaactgaacgccgccgaggccttcgagacgttcctgcagacgaagtatgtcggccagaaacgcttctcgctggagggcgcggagagcgtgatccccatgatggacgccgtcatcgaccaggccgccgagcatcagctggacgaggtggtgatcggcatgccgcatcggggccgcctgaatgtcctggcgaacatcgtgggcaagccgtattccaagatcttcaccgagttcgagggcaacatgaacccggcggcggcccacggcagcggcgacgtgaagtatcacctgggcgccgaaggcacgtatatccagatgttcggcgataacgacatcaccgtctcgctcaccgcgaacccgagccatctggaagccgtggacccggtgctggagggcctggtccgggccaagcaggacatcctcgacaagggcgaagacggcttcacggtgctccccctcatgctccacggcgacgcggccttcgccggccagggcgtggtcgccgagacgctcaacctcgccctgctgcgcggctaccggaccggcggcaccgtgcacatcgtggtgaacaaccaggtcggcttcacgaccgcccccgagtatagccgctccagcgagtactgcacggatgtggcgaagatgatcggcgcccccatcttccacgtcaacggcgatgaccccgaggcgtgcgtgtgggtggcccagctggcggtggacttccgcgagaagttccagaaggacgtggtcatcgacatgatctgttatcggcggcggggccataatgagggcgacgatccgagcatgacccagcccgcgatgtacgacgtgatcgacacgaaacgctccgtccgcaaatcctataccgagtcgctgatcggccgcggcgacatctcgctgaaggaagcggaggacgccctgcgcgactatcagggccagctggaacgggtcttcaatgaggtgcgcgagctggagaagtacacgcccgaaccgagcgagagcgtcgagctggaccaggtcctgcccacgaagctgaaaacctccgtcgatgaatcggtgctggagcgcatcggcgatgccttcgtgaacgtcccggagggcttcacggtccatccgcgggtgaagccggtgatcgaaaagcgccgcgagatgagccgcgaaggcaagatcgactgggccttcgccgagctgctggccttcggctccctggtggatcagggcaagatggtgcgcctctccggccaggacagcaagcgcggcacgttcacccagcgccactcggtcctcatcgaccgcaaaaccggcgccgaatatacccccctccagaacctgggcagcgaaaacccgggcaagttcctggtctacgattccgcgctgagcgagttcgcggcggtcggcttcgagtacggctatagcgtcggcaaccccgacgcgctggtgctgtgggaagcccagttcggcgatttcgtgaatggcgcccagagcatcatcgacgagttcatctcgtcgggcgaagccaaatggggccagctgagcgacgtggtcctgctgctgccccacggccacgaaggccagggcccggaccatacgtccggccgcatcgaacggttcctccagctgtgtgccgaaggctccatgaccgtggcggtccccagcacgccggcctcgtacttccacctgctgcgccggcatagcctcgacggcatccgccggccgctcgtcgtgttcacccccaaaagcatgctgcgcaacaaagccgccgtgagcgacgtggaagacttcacgaccggcaagttccggagcgtcttcgaggagcccacctacgagacgggcgatgccgagcgggacaaggtgcgccgggtcctcctggtgtcgggcaagctgtactgggagctgctggccaagaagcagaaggataaccgcgaggacatcgccatcgtgcgcatcgaacagctgtaccccgtcccctcgcggcggctccgcgagacgctggaccgctaccccaacgccaccgagttccgctgggtgcaggaggagcccgccaaccagggcgcctggcccttcttcggcctcgcgctgcccgaactgctgcccgacaaactgtcgggcatcaagcgcatcagccgccggtccatgtcggccccgtcctccggctcgtccaaggtgcacgcggtcgaacagcaggaaatcatcgacgaggccttcggctga

SEQ ID NO: 9 - protein, 298 aa, Escherichia coli
MAAIAFIGLGQMGSPMASNLLQQGHQLRVFDVNAEAVRHLVDKGATPAANPAQAAKDAEFIITMLPNGDLVRNVLFGENGVCEGLSTDALVIDMSTIHPLQTDKLIADMQAKGFSMMDVPVGRTSANAITGTLLLLAGGTAEQVERATPILMAMGSELINAGGPGMGIRVKLINNYMSIALNALSAEAAVLCEALNLPFDVAVKVMSGTAAGKGHFTTSWPNKVLSGDLSPAFMIDLAHKDLGIALDVANQLHVPMPLGAASREVYSQARAAGRGRQDWSAILEQVRVSAGMTAKVKM

SEQ ID NO: 10 - DNA, 897 bp, Escherichia coli
  source 1..897: mol_type = genomic DNA
atggcggcgatcgcgttcatcggcctcggccagatgggctccccgatggccagcaacctcctgcagcagggccatcagctccgggtgttcgacgtgaacgcggaggcggtccggcatctggtggacaaaggcgcgaccccggccgccaaccccgcccaggcggccaaggacgcggagttcatcatcaccatgctccccaacggcgacctcgtccgcaatgtgctgttcggcgagaacggcgtgtgtgagggcctgtcgaccgacgccctcgtcatcgacatgagcaccatccacccgctgcagacggataagctcatcgccgacatgcaggcgaagggcttcagcatgatggatgtccccgtcggccgcacctccgcgaacgcgatcaccggcaccctgctgctcctggccggcggcaccgccgagcaggtggagcgggccacgcccatcctgatggcgatgggctccgaactcatcaacgcgggcggccccggcatgggcatccgcgtcaagctcatcaacaactacatgtcgatcgcgctgaatgccctctccgcggaagcggcggtgctgtgcgaagcgctgaatctgccgttcgatgtggcggtgaaagtcatgagcggcaccgcggcgggcaagggccacttcaccacctcctggccgaacaaggtgctgtccggcgacctgagcccggcgttcatgatcgatctggcccacaaggatctgggcatcgcgctcgacgtggccaaccagctgcacgtcccgatgccgctcggcgccgcctcccgggaagtgtattcccaggcccgcgccgccggccgcggccggcaggattggtccgccatcctggagcaggtgcgcgtcagcgccggcatgaccgcgaaggtgaagatgtga

SEQ ID NO: 11 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 12 - DNA, 1116 bp, Porphyromonas gingivalis
  source 1..1116: mol_type = genomic DNA
atgcagctgttcaaactcaaaagcgtgacgcaccacttcgatacgttcgcggagttcgccaaggagttctgcctcggcgagcgggacctggtcatcaccaatgagttcatctacgagccctatatgaaggcgtgccagctcccgtgccacttcgtcatgcaggaaaagtacggccagggcgaaccgtcggatgagatgatgaacaacatcctggcggatatccggaacatccagttcgaccgggtcatcggcatcggcggcggcaccgtcatcgacatctccaaactgttcgtcctgaaaggcctcaacgacgtgctggacgccttcgaccgcaaaatcccgctcatcaaggagaaggagctcatcatcgtgcccacgacctgcggcaccggctcggaagtgaccaacatctcgatcgccgagatcaagtcgcgccacaccaagatgggcctggcggatgacgcgatcgtggccgaccatgccatcatcatccccgagctgctgaagagcctgccgttccacttctatgcgtgctccgccatcgatgccctgatccacgccatcgagagctacgtctcgccgaaggcctccccgtacagccggctgttcagcgaggcggcctgggacatcatcctggaagtcttcaagaagatcgccgagcacggccccgaatatcggttcgaaaagctcggcgagatgatcatggccagcaattacgccggcatcgccttcggcaacgccggcgtcggcgccgtccacgccctgtcgtacccgctgggcggcaattatcatgtgccgcatggcgaagcgaattaccagttcttcaccgaggtgttcaaggtctatcagaagaaaaacccgttcggctacatcgtcgagctgaactggaagctgtcgaagatcctgaactgccagccggagtatgtgtatcccaagctggatgaactcctgggctgcctgctgaccaaaaagccgctgcatgaatatggcatgaaggacgaagaggtgcggggcttcgccgaatcggtgctgaagacccagcagcgcctgctcgccaacaactacgtggagctgaccgtcgacgaaatcgaaggcatctaccgccggctgtactga

SEQ ID NO: 13 - protein, 371 aa, Clostridium kluyveri
MKLLKLAPDVYKFDTAEEFMKYFKVGKGDFILTNEFLYKPFLEKFNDGADAVFQEKYGLGEPSDEMINNIIKDIGDKQYNRIIAVGGGSVIDIAKILSLKYTDDSLDLFEGKVPLVKNKELIIVPTTCGTGSEVTNVSVAELKRRHTKKGIASDELYATYAVLVPEFIKGLPYKFFVTSSVDALIHATEAYVSPNANPYTDMFSVKAMELILNGYMQMVEKGNDYRVEIIEDFVIGSNYAGIAFGNAGVGAVHALSYPIGGNYHVPHGEANYLFFTEIFKTYYEKNPNGKIKDVNKLLAGILKCDESEAYDSLSQLLDKLLSRKPLREYGMKEEEIETFADSVIEGQQRLLVNNYEPFSREDIVNTYKKLY

SEQ ID NO: 14 - DNA, 1116 bp, Clostridium kluyveri
  source 1..1116: mol_type = genomic DNA
atgaagctcctgaagctggccccggatgtgtacaaattcgataccgccgaggagttcatgaagtatttcaaggtcggcaagggcgacttcatcctgacgaacgagttcctctataaacccttcctggaaaagttcaatgatggcgccgacgccgtgttccaggaaaagtacggcctgggcgagccgtccgacgaaatgatcaacaacatcatcaaggatatcggcgacaaacagtacaatcggatcatcgcggtgggcggcggcagcgtcatcgatatcgccaaaatcctgtcgctgaaatacacggacgattccctggacctgttcgaaggcaaggtcccgctggtgaagaataaagagctcatcatcgtcccgaccacctgcggcaccggctcggaagtgacgaatgtctcggtcgccgaactgaagcggcgccatacgaagaaaggcatcgcctccgacgaactctatgcgacgtacgcggtcctggtgcccgagttcatcaaaggcctgccctataagttcttcgtgacgagctccgtcgacgccctcatccatgcgaccgaggcctacgtgtcgccgaacgccaacccctacaccgacatgttctcggtcaaggcgatggaactgatcctgaacggctacatgcagatggtggagaagggcaacgattatcgcgtggaaatcatcgaggatttcgtgatcggctccaactacgcgggcatcgcgttcggcaacgcgggcgtcggcgcggtccatgccctcagctaccccatcggcggcaactaccatgtcccgcatggcgaagccaactacctcttcttcacggagatcttcaagacctactacgagaagaacccgaatggcaagatcaaggacgtgaataagctcctggcgggcatcctcaagtgcgatgagtccgaggcgtacgattcgctctcgcagctgctggacaaactgctctcgcgcaagccgctgcgcgagtacggcatgaaggaggaagagatcgaaaccttcgccgacagcgtgatcgaaggccagcagcggctgctggtcaacaactacgagcccttcagccgcgaggatatcgtcaacacctacaaaaagctctactga

SEQ ID NO: 15 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 16 - DNA, 1296 bp, Porphyromonas gingivalis
  source 1..1296: mol_type = genomic DNA
atgaaggacgtgctggccgagtacgcctcgcgcatcgtctcggcggaagaagcggtcaagcacatcaagaacggcgagcgcgtcgcgctctcgcatgccgccggcgtcccgcagtcctgcgtcgatgccctggtccagcaggccgatctcttccagaatgtcgaaatctaccatatgctctgcctgggcgagggcaagtacatggcccccgagatggcgccgcatttccgccatatcacgaatttcgtcggcggcaactcgcggaaagccgtggaggagaaccgggccgacttcatccccgtgttcttctacgaggtccccagcatgatccgcaaagatatcctccacatcgacgtggcgatcgtgcagctgagcatgccggatgagaacggctactgcagcttcggcgtgagctgtgactacagcaagccggccgccgaatccgcccacctcgtcatcggcgagatcaaccggcagatgccgtacgtccacggcgacaacctgatccacatcagcaagctggactacatcgtcatggccgactatccgatctatagcctggcgaaacccaagatcggcgaggtcgaggaggccatcggccggaactgcgccgagctgatcgaagacggcgccaccctccagctgggcatcggcgccatcccggacgccgccctgctcttcctgaaagacaagaaagacctgggcatccacaccgaaatgttcagcgacggcgtcgtcgaactggtccggagcggcgtcatcaccggcaagaaaaaaaccctgcacccgggcaaaatggtcgcgacgttcctgatgggctccgaagatgtctatcatttcatcgacaagaaccccgacgtggagctctacccggtggactatgtgaacgacccgcgcgtcatcgcccagaacgacaacatggtcagcatcaactcctgtatcgagatcgacctcatgggccaggtcgtgagcgaatgtatcggctccaagcagttctcgggcacgggcggccaggtggactacgtccgcggcgcggcgtggtcgaagaacggcaagtcgatcatggccatcccctccaccgccaagaacggcaccgcctcgcgcatcgtgccgatcatcgcggagggcgccgcggtgacgaccctgcggaacgaagtggattacgtggtcacggagtatggcatcgcccagctgaagggcaagtcgctccgccagcgggcggaagcgctgatcgcgatcgcgcacccggacttccgggaggagctgaccaaacacctgcggaaacggttcggctga

SEQ ID NO: 17 - protein, 431 aa, Porphyromonas gingivalis
MQWQELYRQRVCSADEAVVDSLKPGTKVVFGHAAAAPVRFSQAMYRQREKLENITVFHMLYFGDAPHLAPEMRSHVHPTLNFLEGNSRPASRDRRVDFIPCHFHEVPELFRQGFFPLDVAVVQVSTPNEEGYCSFGVSCDYTKAAAECAPVVVAEVNKQMPFIGGENLIHISKLTHIIEVDEPIAEVLPPAISDLELRIGQNCASLIKDGDTLQLGIGGIPDAVLRALEGHKDLGIHTEMFTDGVMRMIRKGIINGKKKTLHPEKVVTSLIFGSKELYDFVNNNPVIECYPVDYINNPDVIGKNDRMVSINSCLEMDLMGQAASESIGYEQFSGSGGQVDFLRGAKRSKGGISIMAFPSTAKKGTESRIVPILKEGACVTTGRNEVDYVVTEYGVARLRGATLRQRAEALTAIAHPDFRPALEEEIRRRFE

SEQ ID NO: 18 - DNA, 1296 bp, Porphyromonas gingivalis
  source 1..1296: mol_type = genomic DNA
atgcagtggcaggaactctaccggcagcgcgtgtgctccgcggatgaagccgtcgtggatagcctgaagcccggcaccaaggtggtcttcggccatgccgccgccgcgcccgtgcggttctcgcaggccatgtatcgccagcgcgaaaagctcgaaaacatcaccgtgttccatatgctctatttcggcgatgccccccacctggcccccgaaatgcgctcccatgtccacccgacgctcaacttcctggaaggcaatagccgccccgcgtcgcgggatcggcgcgtggatttcatcccctgccacttccacgaagtgcccgagctgttccgccagggcttcttccccctcgacgtggcggtggtgcaggtgtcgaccccgaacgaggagggctattgtagcttcggcgtctcgtgtgactatacgaaggccgcggccgaatgtgccccggtcgtggtggccgaggtgaacaagcagatgccgttcatcggcggcgagaacctgatccatatctccaagctgacccatatcatcgaagtggatgagccgatcgccgaggtgctgccccccgcgatcagcgacctggagctgcgcatcggccagaactgcgcgtccctcatcaaggacggcgatacgctccagctgggcatcggcggcatcccggacgcggtgctgcgggccctggaaggccataaagacctcggcatccacacggagatgttcaccgacggcgtgatgcggatgatccggaagggcatcatcaacggcaagaaaaagaccctgcacccggaaaaagtggtgaccagcctgatcttcggcagcaaagaactgtacgacttcgtcaacaacaacccggtgatcgagtgctatccggtcgattatatcaacaatccggatgtcatcggcaagaacgaccgcatggtgagcatcaactcctgcctggaaatggacctgatgggccaggccgcgagcgaaagcatcggctacgaacagttctccggctcgggcggccaggtggatttcctgcggggcgcgaagcggtccaaaggcggcatcagcatcatggccttcccctccacggccaagaagggcacggagtcccgcatcgtgcccatcctgaaggagggcgcgtgcgtcacgaccggccgcaatgaggtggattacgtcgtcaccgagtacggcgtcgcccgcctccggggcgccaccctccggcagcgggccgaggccctgacggccatcgcccatcccgacttccgccccgccctggaggaagagatccgccggcgcttcgagtga

SEQ ID NO: 19 - protein, 438 aa, Clostridium aminobutyricum
MDWKKIYEDRTCTADEAVKSIKSGDRVLFAHCVAEPPVLVEAMVANAAAYKNVTVSHMVTLGKGEYSKPEYKENFTFEGWFTSPSTRGSIAEGHGQFVPVFFHEVPSLIRKDIFHVDVFMVMVSPPDHNGFCCVGVSSDYTMQAIKSAKIVLAEVNDQVPVVYGDTFVHVSNIDKFVETSHPLPEIGLPKIGEVEAAIGKHCASLIEDGSTLQLGIGAIPDAVLSQLKDKKHLGIHSEMISDGVVDLYEAGVIDCSQKSIDKGKMAITFLMGTKRLYDFAANNPKVELKTVDYINHPSVVAQCSKMVCINACLQVDFMGQIVSDSIGTKRFSGVGGQVDFVRGASMSIDGKGKAIIAMPSVAKKKDGSMISKIVPFIDHGAAVTTSRNDADYVVTEYGIAEMKGKSLQDRARALINIAHPDFKDELKAEFEKRFNAAF

SEQ ID NO: 20 - DNA, 1317 bp, Clostridium aminobutyricum
  source 1..1317: mol_type = genomic DNA
atggattggaaaaagatctacgaagatcgcacctgcaccgccgatgaagccgtcaagtccatcaagtccggcgaccgcgtcctgttcgcccactgtgtcgccgaacccccggtgctggtcgaagcgatggtggccaacgccgcggcgtacaagaacgtcaccgtgagccatatggtgaccctgggcaagggcgaatatagcaagcccgagtacaaggagaacttcacgttcgagggctggttcacctccccgtcgacgcggggctcgatcgcggagggccacggccagttcgtgcccgtgttcttccatgaggtgccgtccctgatccgcaaggatatcttccacgtcgacgtgttcatggtcatggtgtccccgcccgatcataacggcttctgctgtgtcggcgtcagcagcgactacacgatgcaggccatcaagtcggccaagatcgtgctggcggaagtcaacgaccaggtgcccgtggtgtatggcgatacgttcgtgcatgtctcgaacatcgataagttcgtggaaacctcgcatccgctcccggaaatcggcctgccgaaaatcggcgaggtggaagcggccatcggcaaacactgtgcctccctgatcgaagacggctcgacgctgcagctgggcatcggcgccatccccgacgccgtgctcagccagctgaaagacaagaagcacctgggcatccactcggagatgatctccgacggcgtcgtggacctgtatgaggcgggcgtgatcgactgctcccagaagtcgatcgacaagggcaaaatggcgatcaccttcctgatgggcaccaagcgcctgtacgacttcgcggcgaacaatccgaaggtggagctgaaaacggtggactacatcaaccatccgtcggtggtggcccagtgctccaagatggtgtgcatcaacgcctgcctgcaggtggatttcatgggccagatcgtgagcgacagcatcggcaccaagcgcttctcgggcgtgggcggccaggtcgacttcgtgcggggcgcctccatgagcatcgacggcaagggcaaggcgatcatcgccatgccgtcggtcgccaagaagaaggacggcagcatgatctcgaaaatcgtccccttcatcgatcacggcgccgccgtgaccacctcgcggaacgacgccgattatgtggtcacggagtacggcatcgccgagatgaaaggcaagtccctccaggaccgggcccgggcgctcatcaacatcgcgcacccggatttcaaggatgagctgaaggcggagttcgagaagcggttcaacgcggcgttctga

SEQ ID NO: 21 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 22 - DNA, 2577 bp, Clostridium acetobutylicum
  source 1..2577: mol_type = genomic DNA
atgaaggtgaccaaccagaaagaactgaagcagaaactgaacgaactgcgggaggcgcagaaaaagttcgccacctatacccaggagcaggtggacaaaatcttcaagcagtgtgcgatcgccgccgccaaagaacggatcaacctggcgaaactcgccgtcgaagaaaccggcatcggcctcgtggaggacaagatcatcaaaaatcacttcgccgccgaatatatctataacaaatacaagaacgagaagacctgcggcatcatcgaccatgacgactcgctgggcatcaccaaagtcgccgagcccatcggcatcgtggccgcgatcgtgcccaccaccaacccgacgagcaccgccatcttcaagtcgctgatctccctgaagacgcgcaatgcgatcttcttcagcccccatccgcgggccaaaaagtcgacgatcgcggcggccaaactgatcctggacgccgcggtcaaggccggcgccccgaaaaatatcatcggctggatcgacgaaccgtccatcgagctgtcccaggacctcatgtccgaagccgacatcatcctggcgaccggcggcccgagcatggtgaaggccgcctattccagcggcaaaccggccatcggcgtcggcgccggcaacacccccgcgatcatcgacgagtccgccgacatcgacatggccgtctcctcgatcatcctgagcaagacctacgataacggcgtcatctgcgcgtcggaacagagcatcctggtcatgaactccatctatgaaaaggtcaaggaagagttcgtgaagcgcggctcgtatatcctgaaccagaatgaaatcgcgaagatcaaggagacgatgttcaagaacggcgccatcaacgccgacatcgtcggcaagagcgcctatatcatcgcgaagatggccggcatcgaagtgccgcagaccaccaagatcctgatcggcgaagtccagtccgtcgaaaagtccgaactgttctcccacgaaaagctgagcccggtgctcgccatgtacaaagtcaaagacttcgacgaggccctcaaaaaggcgcagcggctcatcgaactgggcggcagcggccatacctcgtcgctgtacatcgatagccagaacaacaaggacaaagtcaaagagttcggcctggcgatgaagacgagccgcaccttcatcaacatgccgagcagccagggcgcgtccggcgacctgtacaacttcgccatcgccccgagcttcacgctcggctgcggcacctggggcggcaattcggtgtcgcagaacgtggaaccgaaacatctgctgaacatcaaatccgtcgcggagcgccgcgaaaatatgctctggttcaaggtcccgcagaagatctatttcaaatatggctgcctgcgcttcgccctcaaagaactgaaagacatgaataagaaacgcgccttcatcgtcaccgataaagatctgttcaagctgggctacgtgaacaaaatcacgaaggtcctggatgagatcgatatcaagtacagcatcttcaccgacatcaagtccgatcccaccatcgactccgtcaagaagggcgccaaagagatgctcaacttcgaaccggacaccatcatcagcatcggcggcggctcgccgatggacgcggccaaggtcatgcatctgctctatgaatatccggaggccgagatcgaaaacctggccatcaacttcatggacatccggaaacgcatctgcaacttcccgaagctcggcacgaaagccatcagcgtggccatccccacgaccgcgggcaccggctcggaagccacgccgttcgcggtcatcacgaatgacgaaaccggcatgaaataccccctgacctcctacgagctcacccccaatatggccatcatcgacacggagctcatgctcaacatgccgcggaagctcaccgccgccaccggcatcgatgccctggtgcacgccatcgaagcctacgtgagcgtgatggcgaccgactataccgatgagctggccctccgggccatcaagatgatcttcaagtatctgccgcgggcctacaaaaacggcacgaatgacatcgaggcccgcgaaaagatggcgcatgcgagcaacatcgcgggcatggccttcgcgaacgccttcctgggcgtgtgccatagcatggcgcacaaactgggcgcgatgcatcacgtcccgcatggcatcgcctgtgccgtcctgatcgaggaggtcatcaagtacaacgccacggattgtccgaccaaacagaccgccttcccgcagtacaagtccccgaacgcgaagcggaaatacgcggagatcgcggagtacctgaatctgaaaggcaccagcgacaccgaaaaagtcacggccctcatcgaagccatctcgaaactgaaaatcgatctgagcatcccccagaacatctccgcggccggcatcaataagaaggatttctataacacgctggacaagatgagcgagctggcgttcgatgaccagtgcacgaccgccaacccccggtatccgctgatctccgaactcaaggacatctacatcaagtccttctga

SEQ ID NO: 23 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 24 - DNA, 2676 bp, Escherichia coli
  source 1..2676: mol_type = genomic DNA
atggcggtgacgaacgtcgccgaactgaacgccctcgtcgaacgggtgaaaaaggcgcagcgggagtacgcctcgttcacgcaggagcaggtggataaaatcttccgcgccgccgccctggccgcggccgacgcccgcatcccgctcgccaaaatggcggtggccgagtccggcatgggcatcgtggaggacaaggtcatcaagaaccatttcgcctccgagtacatctacaacgcctacaaggacgagaagacctgcggcgtcctgtcggaggacgataccttcggcaccatcacgatcgccgagcccatcggcatcatctgcggcatcgtcccgaccaccaatcccacctccaccgccatcttcaaaagcctcatctccctgaaaacccgcaatgcgatcatcttcagcccccatccccgcgccaaagacgccacgaacaaggcggccgacatcgtcctccaggccgccatcgcggccggcgcccccaaagacctgatcggctggatcgaccagccgtcggtggagctgtccaacgccctcatgcatcacccggacatcaatctcatcctggccacgggcggcccgggcatggtgaaggccgcctactccagcggcaagccggcgatcggcgtgggcgcgggcaacacgcccgtggtcatcgacgaaacggccgatatcaaacgcgccgtcgcctccgtgctcatgagcaagaccttcgacaacggcgtgatctgcgcgtcggaacagtcggtcgtggtcgtcgactccgtgtacgacgcggtgcgcgagcgcttcgccacgcatggcggctacctgctgcagggcaaggagctgaaagccgtgcaggacgtgatcctcaaaaacggcgccctgaacgccgccatcgtcggccagcccgcgtataagatcgcggagctggccggcttctcggtcccggagaacacgaagatcctgatcggcgaggtcacggtcgtcgacgagagcgaacccttcgcccacgagaagctgtccccgaccctggcgatgtaccgggcgaaagacttcgaagacgcggtcgagaaggccgagaaactggtggcgatgggcggcatcggccatacgagctgtctgtacaccgaccaggacaatcagcccgcccgcgtgtcctacttcggccagaaaatgaagaccgcccgcatcctcatcaatacgcccgcgagccagggcggcatcggcgatctctacaatttcaagctcgcgccgtcgctgaccctcggctgcggctcctggggcggcaactccatctccgagaacgtgggcccgaaacacctcatcaacaagaagaccgtggccaagcgggcggagaacatgctgtggcataagctgcccaagtcgatctacttccgccggggctcgctgcccatcgcgctggacgaagtcatcacggacggccacaagcgggcgctgatcgtgacggaccgcttcctgttcaataacggctacgccgaccagatcacctccgtcctgaaggccgcgggcgtcgaaacggaagtcttcttcgaagtcgaagccgacccgaccctctccatcgtccggaagggcgccgagctggccaattccttcaagccggatgtcatcatcgcgctgggcggcggctcgccgatggacgccgccaaaatcatgtgggtgatgtacgagcaccccgaaacgcacttcgaagaactggcgctgcgcttcatggatatccgcaagcggatctacaaattcccgaaaatgggcgtgaaagcgaagatgatcgcggtgacgaccaccagcggcacgggctccgaggtgacgccgttcgcggtcgtcaccgatgatgccaccggccagaaataccccctggccgactacgccctgacccccgacatggccatcgtggacgccaatctcgtgatggacatgccgaagagcctctgcgccttcggcggcctggatgccgtgacgcatgcgatggaggcgtatgtcagcgtgctggcgtccgagttctcggacggccaggcgctgcaggccctgaagctgctgaaggagtacctgccggcctcctaccacgagggctccaaaaacccggtggcccgggagcgggtccactcggcggccaccatcgcgggcatcgccttcgcgaacgccttcctcggcgtgtgccattcgatggcccacaagctgggctcccagttccatatcccccacggcctggcgaacgcgctgctgatctgtaacgtcatccggtacaacgcgaacgataacccgaccaaacagaccgccttctcccagtatgaccgcccccaggcccggcgccgctatgcggaaatcgccgaccatctcggcctgtcggccccgggcgaccgcaccgccgccaagatcgagaagctgctggcctggctcgaaacgctgaaggccgaactgggcatcccgaagtcgatccgcgaagccggcgtccaggaagcggacttcctggcgaatgtggataaactgagcgaggacgccttcgatgaccagtgcacgggcgccaacccgcgctacccgctcatcagcgagctgaaacagatcctgctcgacacctattatggccgcgattacgtcgagggcgaaaccgccgcgaagaaggaggccgccccggccaaagccgaaaagaaggccaagaaaagcgcctga

SEQ ID NO: 25 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWGATQALNGLIGAGVPQDWATHMLGHELTAMHGLDHAQTLAIVLPALWNEKRETKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 26 - DNA, 1164 bp, Escherichia coli
  source 1..1164: mol_type = genomic DNA
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 27 - protein, 383 aa, Escherichia coli
MMANRMILNETAWFGRGAVGALTDEVKRRGYQKALIVTDKTLVQCGVVAKVTDKMDAAGLAWAIYDGVVPNPTITVVKEGLDVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTDEKYRDIARVMGVKVEGMSLEEARNAAVEAVFALNRDVGIPPHLRDVGVRKEDIPALAQAALDDVCTGGNPREATLEDIVELYHTAW

SEQ ID NO: 28 - DNA, 1149 bp, Escherichia coli
  source 1..1149: mol_type = genomic DNA
atggcgaatcggatgatcctcaatgaaacggcctggttcggccgcggcgcggtcggcgccctcaccgatgaggtcaagcggcggggctaccagaaggccctgatcgtcacggataaaaccctggtgcagtgcggcgtcgtcgccaaggtgaccgacaagatggatgcggccggcctggcctgggcgatctacgacggcgtggtgcccaaccccaccatcaccgtggtgaaggaaggcctgggcgtgttccagaactcgggcgcggattatctcatcgcgatcggcggcggcagcccccaggacacctgcaaggccatcggcatcatctcgaacaaccccgagttcgcggacgtgcgctccctggagggcctgtcgccgacgaacaagccctccgtcccgatcctcgccatcccgacgacggccggcaccgcggccgaggtgaccatcaattacgtcatcaccgacgaggaaaagcggcgcaagttcgtgtgtgtggacccccatgacatcccccaggtcgccttcatcgacgccgacatgatggatggcatgccccccgccctcaaggccgcgacgggcgtggacgcgctgacgcatgccatcgaaggctacatcacccggggcgcctgggccctgacggatgccctgcatatcaaggccatcgaaatcatcgccggcgccctgcgcggctccgtggccggcgacaaggatgcgggcgaggagatggcgctgggccagtacgtggccggcatgggcttctccaatgtgggcctgggcctggtgcatggcatggcccatccgctcggcgccttctacaacacgccgcatggcgtcgcgaacgcgatcctcctgccgcatgtcatgcgctacaatgcggacttcacgggcgagaaataccgcgatatcgcccgggtcatgggcgtgaaggtcgagggcatgtcgctggaagaggcgcggaacgccgcggtcgaagccgtcttcgccctgaaccgggatgtgggcatcccgccgcacctgcgcgatgtcggcgtccgcaaggaagacatccccgcgctggcgcaggccgccctggacgatgtgtgcaccggcggcaacccccgcgaggcgacgctggaagacatcgtcgaactctaccataccgcgtggtga

SEQ ID NO: 29 - protein, 860 aa, Clostridium beijerinckii
MRVTNPEELTKRIEQIREAQREFAKFSQEEVDEIFRQAAMAANDARITLAKMAVEESGMGIVEDKVIKNHFAAEYIYNQYKDTKTCGVIERDEMFGITHIAEPIGVIAAIVPTTNPTSTAIFKTLIALKTRNGIIISPHPRAKNSTIAAAKIVLEAAERAGAPKGIIGWIDEPSIELSRNVMSESDIILATGGPGMVRAAYSSGKPAIGVGAGNTPAIIDDTAHIKMAVNSILLSKTFDNGVVCASEQSIIAMESVYDEVRKELDERGAYILKGDEVDKVRSIILDPKGSLNSEIVGQSAYKIAKMAGVEVSEAVKVLIGEVESPELEEPFSHEKLSPILGMYKAKTFDDALRLASRMIELGGFGHTSILYTNQVESVDRIEKFGVAMKTARTLINMPASQGAIGDIYNFKLAPSLTLGCGSWGGNSISENVGPKHLINVKRIAERRENMLWFRVPDKIYFKFGCLPIALEELNAMKKKRAFIVTDRVLFDLGYTHKITDILSENHIEYKIFSDVEPDPTLKAAKLGADAMRDFNPDVIIAIGGGSPMDAAKIMWVMYEHPDVRFEDLAMRFMDIRKRVYEFPPMGERAILVAIPTSAGTGSEVTPFAVITDQQTGVKYPLADYALTPNMAIIDAELMMSMPKGLTAASGIDALVHAIEAYVSVLASEYTNGLALEAIRLTFKYLPDAYNGGTTNIKAREKMAHASSVAGMAFANAFLGICHSMAHKLGAFHHVPHGIANALLIDEVIRFNATDAPRKQAAFPQYKYPNAGWRYARIADYLNLGGNTEEEKVELLIKAIDDLKVKVRIPKSIKEFGVSEEKFYDSMDEMVEQAFDDQCTGANPRYPLMSEIKEMYIKSYN

SEQ ID NO: 30 - DNA, 1407 bp, Clostridium beijerinckii
  source 1..1407: mol_type = genomic DNA
atgaacaaggacacgctgatccccaccacgaaagacctcaaggtgaagaccaacggcgagaacatcaacctgaaaaactacaaggacaacagcagctgcttcggcgtgttcgaaaacgtcgaaaacgccatcagctcggcggtgcatgcccagaaaatcctcagcctgcactatacgaaggaacagcgcgaaaagatcatcaccgaaatccgcaaggccgcgctccagaacaaagaggtgctggccacgatgatcctggaggaaacccatatgggccgctacgaagataaaatcctgaagcacgagctggtggcgaaatacacgcccggcaccgaagacctgacgaccaccgcctggagcggcgataacggcctgacggtggtggagatgtcgccgtatggcgtgatcggcgcgatcaccccgtccaccaacccgaccgaaacggtcatctgtaactcgatcggcatgatcgcggccggcaacgcggtcgtgttcaatggccacccgtgcgccaagaagtgcgtcgccttcgccgtcgagatgatcaacaaggccatcatctcctgcggcggcccggagaacctcgtgacgacgatcaaaaacccgacgatggagtccctggatgccatcatcaagcacccgtccatcaagctgctctgcggcaccggcggcccgggcatggtcaaaacgctgctgaactccggcaaaaaagccatcggcgcgggcgcgggcaacccccccgtcatcgtggatgacaccgccgacatcgagaaagccggccggtcgatcatcgaaggctgctccttcgacaataacctcccgtgcatcgccgagaaagaggtcttcgtcttcgagaacgtcgcggacgatctgatctccaacatgctgaagaacaacgccgtcatcatcaacgaagaccaggtgtccaagctgatcgacctcgtgctgcagaagaacaatgaaacccaggagtacttcatcaacaagaagtgggtcggcaaggacgcgaagctgttcctggacgaaatcgatgtggagtccccgagcaatgtgaagtgcatcatctgcgaggtcaacgcgaaccaccccttcgtcatgaccgagctcatgatgccgatcctgccgatcgtgcgggtgaaggatatcgacgaagccatcaagtacgcgaaaatcgcggaacagaatcggaagcattcggcctatatctacagcaagaacatcgataacctcaaccgcttcgaacgggagatcgacaccaccatcttcgtcaagaacgccaaatccttcgccggcgtgggctatgaggcggaaggcttcaccaccttcacgatcgccggctccaccggcgagggcatcacctccgcccggaacttcacccgccagcgccgctgcgtgctggcgggctga

SEQ ID NO: 31 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 32 - DNA, 1083 bp, Saccharomyces cerevisiae
  source 1..1083: mol_type = genomic DNA
atgagctatcccgagaagttcgaggggatcgccatccagagccacgaggactggaagaacccgaaaaagaccaagtatgatccgaagcccttctacgatcacgacatcgacatcaagatcgaggcctgcggcgtctgcggcagcgatatccattgtgcggctggccactggggcaacatgaagatgccgttggtcgtcggccacgagatcgtgggcaaggtcgtgaagttaggcccgaaaagcaacagcggcttgaaggtgggccagcgcgtgggtgtgggtgcgcaggtcttcagctgtctggagtgcgaccgttgcaagaacgacaacgaaccgtactgcaccaagttcgtcaccacctactcgcagccctacgaggacggctacgtctcgcagggcggttacgccaactatgtccgagtccacgaacacttcgtggtgcccatcccggaaaatatccccagccatctggcggctcccctgctgtgcggtggcttgaccgtctacagccccctcgtccgcaatggctgcggtcccggcaagaaggtgggtatcgtgggcctcggcggtataggctctatgggcacgctgatctcgaaagcgatgggcgcagaaacgtacgtgatctcgcgttcctcgcgcaagcgcgaggatgcgatgaagatgggtgcggaccactacatcgccacgctggaggagggtgactggggtgagaagtacttcgacacgttcgacctcatcgtggtgtgcgcgagttccctgacggacatcgacttcaatatcatgcccaaggcgatgaaggtcggagggcgcatcgtctccatctcgatcccggagcagcacgaaatgctgtcgctgaagccctacggcctgaaagccgtctccattagctactcggcgctcggtagtatcaaggagctcaaccagctgttgaagttggtttccgaaaaggacatcaagatctgggtggaaacgctcccggtgggcgaagccggtgtgcacgaggcctttgagcggatggagaagggggatgtccgttatcggtttacactcgtcggctacgataaagagttctcggattaa

SEQ ID NO: 33 - protein, 340 aa, Acinetobacter baylyi
MTTNVIHAYAAMQAGEALVPYSFDAGELQPHQVEVKVEYCGLCHSDVSVLNNEWHSSVYPVVAGHEVIGTITQLGSEAKGLKIGQRVGIGWTAESCQACDQCISGQQVLCTGENTATIIGHAGGFADKVRAGWQWVIPLPDELDPTSAGPLLCGGITVFDPILKHQIQAIHHVAVIGIGGLGHMAIKLLKAWGCEITAFSSNPNKTDELKAMGADHVVNSRDDAEIKSQQGKFDLLLSTVNVPLNWNAYLNTLAPNGTFHFLGVVMEPIPVPVGALLGGAKSLTASPTGSPAALRKLLEFAARKNIAPQIEMYPMSELNEAIERLHSGQARYRIVLKADF

SEQ ID NO: 34 - DNA, 1023 bp, Acinetobacter baylyi
  source 1..1023: mol_type = genomic DNA
atgacgaccaacgtcatccatgcctatgcggccatgcaggccggcgaggcgctggtgccgtatagcttcgacgcgggcgagctccagccgcatcaggtcgaggtgaaggtggaatactgcggcctgtgtcattccgatgtgtcggtcctgaataacgaatggcactcctccgtctatccggtggtcgccggccacgaggtcatcggcacgatcacccagctgggcagcgaagccaagggcctgaagatcggccagcgcgtcggcatcggctggacggccgagagctgccaggcctgcgatcagtgcatctccggccagcaggtgctgtgcaccggcgagaacaccgccaccatcatcggccatgccggcggcttcgcggataaagtgcgggccggctggcagtgggtgatcccgctcccggatgagctcgatcccacgagcgccggcccgctgctgtgtggcggcatcaccgtgttcgacccgatcctgaagcaccagatccaggccatccaccatgtcgccgtgatcggcatcggcggcctgggccacatggcgatcaagctgctcaaggcctggggctgtgaaatcaccgccttcagcagcaatcccaacaagaccgacgaactgaaggcgatgggcgccgaccatgtggtcaactcccgcgatgacgcggaaatcaagagccagcagggcaagttcgacctgctgctgtcgacggtgaatgtccccctcaactggaacgcctacctgaataccctggccccgaacggcacgttccacttcctgggcgtcgtcatggagccgatcccggtgccggtgggcgccctcctgggcggcgcgaaaagcctgaccgcctcgcccacgggcagccccgccgccctccgcaagctgctggagttcgccgcccgcaagaacatcgcgccccagatcgaaatgtatcccatgagcgagctgaacgaagccatcgagcgcctgcatagcggccaggcccgctatcggatcgtgctcaaagcggacttctga

SEQ ID NO: 35 - protein, 339 aa, Escherichia coli
MSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 36 - DNA, 1020 bp, Escherichia coli
  source 1..1020: mol_type = genomic DNA
atgtccatgatcaaaagctacgccgcgaaagaggcgggcggcgagctggaggtgtatgagtatgacccgggcgagctgcggccccaggacgtggaagtgcaggtcgactactgcggcatctgccattcggacctctcgatgatcgataacgagtggggcttcagccagtaccccctggtggccggccacgaggtgatcggccgcgtggtcgccctgggctcggccgcgcaggataaaggcctgcaggtcggccagcgcgtcggcatcggctggacggcccgcagctgcggccattgcgatgcctgcatcagcggcaatcagatcaattgcgaacagggcgcggtcccgaccatcatgaaccggggcggcttcgccgaaaagctgcgggccgattggcagtgggtgatcccgctgccggagaacatcgatatcgagtcggccggccccctgctgtgcggcggcatcaccgtcttcaagccgctcctgatgcatcatatcacggcgaccagccgggtcggcgtgatcggcatcggcggcctcggccacatcgcgatcaaactgctgcacgcgatgggctgcgaggtcaccgcgttctcctcgaaccccgccaaggagcaggaagtgctggcgatgggcgccgataaagtcgtgaactcgcgcgacccccaggccctcaaagccctggccggccagttcgatctcatcatcaacacggtgaacgtgtcgctggactggcagccctacttcgaagccctgacctatggcggcaacttccataccgtcggcgccgtgctgaccccgctgtccgtcccggccttcaccctgatcgccggcgaccgcagcgtgtccggcagcgccaccggcacgccgtatgagctgcgcaagctgatgcgcttcgccgcccgcagcaaggtcgccccgaccaccgagctgttccccatgtccaagatcaatgacgcgatccagcatgtccgggacggcaaggcccgctatcgcgtcgtcctcaaggcggacttctga

SEQ ID NO: 37 - protein, 388 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 38 - DNA, 1167 bp, Escherichia coli
  source 1..1167: mol_type = genomic DNA
atgaacctgcatgaatatcaggccaagcagctgttcgcgcggtatggcctcccggcgccggtcggctacgcctgtacgaccccgcgggaagcggaggaggccgcctccaagatcggcgccggcccgtgggtggtcaaatgccaggtgcatgcgggcggccggggcaaggcgggcggcgtgaaggtcgtcaactccaaggaggacatccgcgccttcgccgagaactggctgggcaagcggctggtgacctatcagacggacgccaatggccagcccgtcaatcagatcctggtcgaggcggccacggacatcgcgaaagaactgtacctcggcgccgtcgtggaccggagcagccggcgggtggtgttcatggcgtccaccgagggcggcgtggaaatcgaaaaagtggccgaggaaaccccgcacctgatccataaagtcgcgctggacccgctgaccggccccatgccgtatcagggccgggaactcgcgttcaagctcggcctggagggcaagctggtgcagcagttcacgaaaatcttcatgggcctggcgaccatcttcctggagcgcgacctggccctgatcgaaatcaacccgctggtcatcacgaagcagggcgacctgatctgcctggacggcaagctcggcgccgacggcaacgccctgttccgccagccggacctgcgggaaatgcgcgatcagtcgcaggaggacccccgggaggcccaggcggcccagtgggagctgaattatgtggcgctcgatggcaatatcggctgcatggtcaatggcgcgggcctggcgatgggcacgatggacatcgtgaagctgcatggcggcgagcccgccaacttcctggacgtgggcggcggcgcgaccaaagagcgggtgacggaagcgttcaagatcatcctgagcgacgataaagtcaaggccgtgctggtcaacatcttcggcggcatcgtccgctgcgacctgatcgccgacggcatcatcggcgcggtggcggaggtcggcgtcaatgtgccggtcgtggtccgcctggagggcaacaacgccgaactgggcgccaagaagctggccgattccggcctgaacatcatcgcggcgaagggcctgaccgatgccgcgcagcaggtcgtggccgccgtcgagggcaagtga

SEQ ID NO: 39 - protein, 289 aa, Escherichia coli
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 40 - DNA, 870 bp, Escherichia coli
  source 1..870: mol_type = genomic DNA
atgtccatcctcatcgacaagaacaccaaggtgatctgccagggcttcaccggctcccagggcaccttccacagcgagcaggcgatcgcctacggcaccaagatggtgggcggcgtcacccccggcaagggcggcaccacccatctcggcctcccggtgttcaatacggtgcgggaagccgtggcggccaccggcgcgaccgccagcgtcatctatgtgcccgcgccgttctgcaaggactccatcctggaagccatcgacgccggcatcaagctcatcatcaccatcaccgagggcatccccaccctcgacatgctgacggtgaaagtcaagctggacgaggcgggcgtccggatgatcggccccaactgcccgggcgtcatcacccccggcgagtgcaaaatcggcatccagccgggccacatccacaaaccgggcaaggtgggcatcgtctcgcgctccggcaccctcacctatgaagccgtcaagcagaccaccgactatggcttcggccagtcgacctgcgtcggcatcggcggcgaccccatcccgggctcgaacttcatcgacatcctggagatgttcgagaaagacccccagaccgaggccatcgtgatgatcggcgagatcggcggctcggcggaggaggaggcggcggcgtacatcaaagagcatgtgaccaagccggtcgtgggctatatcgcgggcgtgacggcgcccaagggcaagcgcatgggccatgcgggcgccatcatcgcgggcggcaaaggcaccgcggatgagaagttcgccgccctggaggccgccggcgtcaagaccgtccgcagcctcgccgatatcggcgaagccctgaagaccgtgctcaagtga

SEQ ID NO: 41 - protein, 453 aa, Clostridium kluyveri
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 42 - DNA, 1362 bp, Clostridium kluyveri
  source 1..1362: mol_type = genomic DNA
atgtcgaatgaagtctcgatcaaggaactgatcgaaaaggcgaaggtggcccagaagaaactggaagcctactcgcaggagcaggtggatgtcctggtcaaggccctgggcaaggtcgtgtatgataacgcggagatgttcgccaaagaagcggtggaagaaacggaaatgggcgtgtacgaggacaaggtggcgaagtgccacctgaaatccggcgccatctggaaccatatcaaggacaagaaaaccgtgggcatcatcaaggaagagccggagcgggccctggtctacgtcgccaagcccaagggcgtggtggccgccaccacgcccatcaccaaccccgtcgtcacgccgatgtgtaatgcgatggccgcgatcaagggccgcaatacgatcatcgtggccccgcaccccaaagcgaaaaaggtgtccgcccacaccgtcgagctgatgaacgccgagctgaagaagctgggcgccccggagaacatcatccagatcgtggaagcgccctcgcgggaggcggcgaaggaactcatggagagcgccgacgtggtcatcgccaccggcggcgcgggccgggtgaaggccgcctactcgtccggccgccccgcgtacggcgtcggcccgggcaactcgcaggtcatcgtcgacaagggctatgactacaacaaggccgcgcaggacatcatcaccggccgcaagtatgacaacggcatcatctgctcctccgagcagagcgtgatcgcgcccgccgaagactatgacaaggtgatcgccgccttcgtcgagaatggcgcgttctacgtggaggatgaagaaaccgtggaaaagttccgctccaccctgttcaaagacggcaagatcaattccaagatcatcggcaagagcgtccagatcatcgccgacctggcgggcgtcaaggtccccgagggcacgaaggtcatcgtgctgaagggcaagggcgcgggcgagaaggatgtcctgtgcaaggaaaagatgtgcccggtgctggtggccctgaagtacgacaccttcgaagaggccgtcgaaatcgcgatggccaactatatgtatgaaggcgccggccacaccgccggcatccattcggacaacgacgagaacatccgctacgcgggcaccgtgctccccatcagccgcctcgtggtgaaccagccggccaccaccgccggcggcagcttcaataacggcttcaaccccaccaccaccctgggctgcggcagctggggccgcaacagcatctcggaaaacctcacgtacgaacatctcatcaacgtctcccgcatcggctatttcaacaaggaagcgaaggtgccctcctatgaagagatctggggctga

SEQ ID NO: 43 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 44 - DNA, 1356 bp, Porphyromonas gingivalis
  source 1..1356: mol_type = genomic DNA
atggagatcaaggaaatggtgtccctggcccgcaaggcccagaaggagtaccaggccacccataatcaggaagccgtggacaacatctgccgcgccgcggcgaaagtcatctacgaaaatgccgccatcctcgcccgcgaggcggtcgatgaaaccggcatgggcgtgtacgaacacaaggtggccaagaatcagggcaaaagcaagggcgtgtggtacaacctgcataacaagaaaagcatcggcatcctgaacatcgacgagcgcaccggcatgatcgagatcgccaagcccatcggcgtcgtcggcgcggtcacgccgaccaccaatcccatcgtcaccccgatgtccaacatcatcttcgcgctgaaaacctgcaacgcgatcatcatcgccccccacccgcgcagcaaaaaatgctcggcccacgccgtgcggctcatcaaggaagccatcgcgccgttcaacgtgccggagggcatggtgcagatcatcgaagagccgtcgatcgagaagacccaggaactgatgggcgccgtcgacgtggtggtggccaccggcggcatgggcatggtgaagtcggcgtatagctcgggcaagccgagcttcggcgtgggcgccggcaacgtgcaggtcatcgtcgattcgaacatcgatttcgaagccgccgcggaaaagatcatcacgggccgcgcgttcgataacggcatcatctgctccggcgaacagagcatcatctacaacgaggcggataaggaggccgtcttcacggccttccggaaccacggcgcgtacttctgcgatgaagccgaaggcgatcgcgcccgggcggcgatcttcgaaaacggcgccatcgccaaagacgtggtgggccagagcgtggcgttcatcgccaagaaagcgaacatcaatatccccgaaggcacgcgcatcctggtggtggaagcccgcggcgtcggcgcggaagatgtgatctgcaaggaaaagatgtgccccgtgatgtgcgccctgtcctacaaacacttcgaagagggcgtcgagatcgcccgcaccaacctcgcgaacgagggcaacggccacacctgcgccatccattcgaataatcaggcccacatcatcctggccggcagcgagctcaccgtcagccgcatcgtggtcaacgcgccctccgccaccaccgccggcggccatatccagaacggcctggccgtgacgaacaccctgggctgcggctcgtggggcaacaacagcatctcggaaaacttcacctataagcatctgctcaatatcagccgcatcgccccgctgaacagcagcatccatatcccggacgacaaagagatctgggaactctga

SEQ ID NO: 45 - protein, 469 aa, Methylococcus capsulatus
MSQDDYRIEKDSMGELRVPVSALYAAQTQRAIDNFPVSGLALPPAFIRAIARIKQCAARVNVALGHLDAGKGEAIMAAAEEIIGGAYADQFPVDVFQTGSGTSTNMNVNEVLATLASRRAGTPVSANDDVNMGQSSNDVIPTAIHVSAALAVNEHLIPALEHLAATIRYKGLAHPKAVKTGRTHLMDAMPVRLEQELSGWALQVANGVDRLRASLPRLYKLGQGGTAVGTGINADPAFASSFAEALADATGLPFRPNDSFFESLSCQDAAVELSGQLKTIAVGIMKIANDLRWMNSGPLAGLGEIELPALQPGSSIMPGKVNPVIPEAACMVAAQVIGNDAAITVAGQSGSFQLNVMLPVIAYNLLQSIELLANIARLLADKAIADFRVREDNLRRALATNPILVTALNPVIGYLKAAEIAKTAYRTGRPILEVAIEMSGLDRAELERLLDPAALTTGGIHGVPAGAAG

SEQ ID NO: 46 - DNA, 1410 bp, Methylococcus capsulatus
  source 1..1410: mol_type = genomic DNA
atgagccaagacgactatcgcatcgaaaaagacagcatgggtgagctgcgggttccggtttccgcgctctacgcggctcagacccagcgtgccatcgacaattttccggtgagcgggctggctttgccgccggcgttcatccgcgcgatcgcccggatcaagcagtgcgcggcccgggtcaatgtggcgctggggcatctggatgcaggcaagggcgaggcgatcatggcggcggccgaggagatcatcggcggggcatacgccgaccagtttcctgtcgacgtgttccagaccggttccggcaccagcaccaacatgaacgtcaacgaagtgctggcgactttggcttcccggcgcgcgggaacgcccgtgagcgccaacgacgacgtcaacatggggcagagcagcaatgacgtcatccccacggcgattcacgtcagtgccgcgctggcagtcaacgaacatctgatcccggcgctggaacacctggcggcgacgatacgctacaagggcttggcgcatccgaaggcggtcaagaccgggcggacccatctgatggacgccatgccagtgcggctggagcaagaactctcgggctgggcgctgcaggtcgccaacggcgtcgaccgcctgcgggcgagtctgccgcggctttacaaattggggcagggcggcacggccgtgggcactggcatcaacgccgatccggccttcgcttcctccttcgccgaagctctggccgatgccacgggcctgccttttcgccccaacgattcgttcttcgagagcctgagttgccaggacgcggcagtcgaactgtcgggccagctcaagaccatcgccgtcggcatcatgaagatcgccaacgacctgcgctggatgaattcgggtcccctggctggcctgggggagatcgagctgcccgcgctccagcccggcagctccatcatgcccggcaaggtgaatccggtgattcccgaagccgcctgcatggtcgcggcgcaggtcatcggcaacgacgccgccatcacggtggctggtcagtcgggttctttccaactcaacgtgatgctgccggtgatcgcctacaacttgctgcagagcatcgagttgctggccaacattgctcgtctgctggccgacaaggccatcgccgacttccgggtgcgtgaggacaatctgcgccgggctctggcaacgaatccgatcctggtcaccgcactcaacccggtgatcggctacctgaaggcggcggaaatcgccaagaccgcctaccggacggggcggccgattctggaggtggccatcgaaatgagcggtctggaccgcgccgaactggagcgactgctcgatccggcggccctgaccaccggcggcatccacggtgtacccgccggcgcggcgggctga

SEQ ID NO: 47 - protein, 467 aa, Escherichia coli
MNTVRSEKDSMGAIDVPADKLWGAQTQRSLEHFRISTEKMPTSLIHALALTKRAAAKVNEDLGLLSEEKASAIRQAADEVLAGQHDDEFPLAIWQTGSGTQSNMNMNEVLANRASELLGGVRGMERKVHPNDDVNKSQSSNDVFPTAMHVAALLALRKQLIPQLKTLTQTLNEKSRAFADIVKIGRTHLQDATPLTLGQEISGWVAMLEHNLKHIEYSLPHVAELALGGTAVGTGLNTHPEYARRVADELAVITCAPFVTAPNKFEALATCDALVQAHGALKGLAASLMKIANDVRWLASGPRCGIGEISIPENEPGSSIMPGKVNPTQCEALTMLCCQVMGNDVAINMGGASGNFELNVFRPMVIHNFLQSVRLLADGMESFNKHCAVGIEPNRERINQLLNESLMLVTALNTHIGYDKAAEIAKKAHKEGLTLKAAALALGYLSEAEFDSWVRPEQMVGSMKAGR

SEQ ID NO: 48 - DNA, 1404 bp, Escherichia coli
  source 1..1404: mol_type = genomic DNA
atgaacaccgtgcggagcgaaaaggacagcatgggcgccatcgacgtgcccgccgataagctgtggggcgcccagacccagcggtccctcgaacacttccggatctcgaccgaaaagatgccgaccagcctgatccatgcgctcgccctgacgaaacgggccgccgccaaggtcaacgaggacctcggcctgctgagcgaagagaaggcctccgcgatccgccaggccgccgatgaagtcctcgccggccagcacgacgacgagttcccgctggccatctggcagaccggctcgggcacccagtcgaacatgaacatgaacgaagtgctcgccaaccgcgcgtcggaactgctgggcggcgtccggggcatggaacgcaaggtccatcccaacgacgacgtgaacaagtcgcagagctccaatgatgtcttccccaccgccatgcatgtcgcggccctcctggccctgcgcaaacagctgatcccccagctgaaaaccctgacccagaccctgaatgaaaaatcccgcgcgttcgccgacatcgtgaagatcggccggacgcacctccaggacgcgacccccctgaccctcggccaggagatctccggctgggtggcgatgctggaacacaacctgaaacatatcgagtacagcctgccgcacgtcgcggaactggcgctgggcggcacggcggtcggcaccggcctgaacacgcatccggagtatgcgcggcgcgtggccgacgaactggccgtcatcacctgtgccccgttcgtgaccgccccgaataaattcgaagccctggcgacctgtgacgccctggtccaggcgcatggcgccctcaagggcctggccgccagcctcatgaaaatcgcgaatgacgtgcgctggctggcctccggcccccgctgcggcatcggcgaaatctcgatccccgaaaacgagccgggctcgtcgatcatgccgggcaaggtgaacccgacccagtgcgaggcgctcacgatgctctgctgccaggtcatgggcaatgacgtggccatcaatatgggcggcgcgtccggcaacttcgaactgaatgtcttccgcccgatggtcatccacaatttcctgcagtccgtgcgcctgctggcggacggcatggagagcttcaataagcactgcgcggtgggcatcgagcccaaccgcgagcgcatcaaccagctgctgaatgagagcctgatgctggtcaccgcgctgaatacccacatcggctacgacaaggcggccgaaatcgccaagaaagcgcacaaagaaggcctgacgctgaaagccgccgccctggccctgggctacctgtccgaggccgagttcgacagctgggtccgcccggagcagatggtgggctcgatgaaagccggccggtga

SEQ ID NO: 49 - protein, 474 aa, Mycobacterium tuberculosis
MAVDADSANYRIEHDTMGEVRVPAKALWRAQTQRAVENFPISGRGLERTQIRALGLLKGACAQVNSDLGLLAPEKADAIIAAAAEIADGQHDDQFPIDVFQTGSGTSSNMNTNEVIASIAAKGGVTLHPNDDVNMSQSSNDTFPTATHIAATEAAVAHLIPALQQLHDALAAKALDWHTVVKSGRTHLMDAVPVTLGQEFSGYARQIEAGIERVRACLPRLGELAIGGTAVGTGLNAPDDFGVRVVAVLVAQTGLSELRTAANSFEAQAARDGLVEASGALRTIAVSLTKIANDIRWMGSGPLTGLAEIQLPDLQPGSSIMPGKVNPVLPEAVTQVAAQVIGNDAAIAWGGANGAFELNVYIPMMARNILESFKLLTNVSRLFAQRCIAGLTANVEHLRRLAESSPSIVTPLNSAIGYEEAAAVAKQALKERKTIRQTVIDRGLIGDRLSIEDLDRRLDVLAMAKAEQLDSDRL

SEQ ID NO: 50 - DNA, 1425 bp, Mycobacterium tuberculosis
  source 1..1425: mol_type = genomic DNA
atggcggtggatgcggacagcgcgaactaccgcatcgaacacgatacgatgggcgaagtgcgcgtccccgccaaggccctctggcgggcccagacgcagcgggccgtcgagaacttcccgatcagcggccggggcctggaacgcacgcagatccgcgccctgggcctcctgaagggcgcctgcgcccaggtgaactccgatctgggcctgctcgcgccggaaaaggcggatgccatcatcgccgccgcggcggagatcgccgacggccagcacgacgatcagttcccgatcgatgtcttccagaccggcagcggcacctccagcaatatgaacaccaacgaggtcatcgcgagcatcgcggccaagggcggcgtgaccctgcacccgaacgacgacgtgaatatgagccagtcgtccaacgacaccttccccaccgccacccatatcgcggcgaccgaggcggccgtcgcgcatctgatccccgccctgcagcagctccacgatgccctggccgcgaaagcgctggactggcacaccgtcgtgaaatccggccggacgcatctcatggacgccgtgccggtgaccctgggccaggagttctccggctacgcccggcagatcgaagccggcatcgaacgggtccgcgcgtgcctcccgcggctgggcgagctggccatcggcggcaccgccgtgggcaccggcctcaacgcgcccgacgatttcggcgtgcgggtggtggcggtgctcgtggcccagaccggcctgtcggaactccggacggcggccaattccttcgaagcccaggccgcccgcgatggcctggtggaggcgtcgggcgccctgcgcacgatcgccgtgagcctgacgaagatcgcgaacgacatccggtggatgggctccggcccgctcaccggcctggcggaaatccagctcccggacctgcagcccggcagctccatcatgcccggcaaggtcaaccccgtgctgccggaagcggtcacccaggtcgcggcccaggtgatcggcaacgacgccgccatcgcctggggcggcgcgaacggcgcgttcgaactgaatgtctatatcccgatgatggcccggaacatcctggagtcgttcaagctgctcaccaacgtgagccggctgttcgcgcagcgctgcatcgccggcctcaccgccaacgtcgaacatctgcggcggctcgcggaatcctcgcccagcatcgtcacgccgctcaacagcgcgatcggctacgaagaagcggcggccgtggccaagcaggcgctcaaagagcgcaagaccatccgccagacggtgatcgaccggggcctgatcggcgatcggctgtcgatcgaggacctggatcgccgcctggatgtgctcgcgatggccaaagccgaacagctggactccgaccgcctgtga

SEQ ID NO: 51 - protein, 602 aa, Escherichia coli
MQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 52 - DNA, 1809 bp, Escherichia coli
  source 1..1809: mol_type = genomic DNA
atgcagaccttccaggccgatctggccatcgtgggtgcaggcggtgcaggtctccgggcagcaatcgctgcagcccaggcgaaccccaacgccaagatcgcgctcatctccaaggtctacccgatgcggtcccataccgtggcggcagagggcggttcggcggcagtggcgcaggaccacgattcgttcgaataccacttccacgacacggtggccggcggcgactggctgtgcgaacaggatgtggtggactacttcgtgcatcattgccccaccgaaatgacccagctggaactgtggggctgcccgtggtcgcgccggcccgatggctccgtgaatgtgcgccggttcggcggcatgaagatcgaacgcacctggttcgcggccgacaagacgggcttccacatgctccataccctgttccagacgagcctgcagttcccccagatccagcggttcgacgagcacttcgtcctggacatcctggtggacgatggccatgtgcgcggcctcgtcgccatgaacatgatggagggcaccctcgtgcagatccgcgccaacgccgtggtcatggcgaccggcggcgccggccgcgtgtaccgctacaataccaacggcggcatcgtgaccggcgacggcatgggcatggccctcagccacggcgtgcccctccgcgacatggagttcgtgcagtaccaccccaccggcctcccgggcagcggcatcctgatgaccgagggctgccggggcgaaggcggcatcctcgtcaacaagaacggctatcgctacctgcaggactacggcatgggcccggaaaccccgctcggcgagcccaagaataaatatatggagctgggcccccgggataaggtcagccaggcgttctggcacgaatggcggaagggcaacaccatctccaccccccgcggcgatgtcgtgtacctcgacctgcggcatctgggcgaaaaaaagctccacgaacgcctgccgttcatctgcgagctggccaaggcctacgtgggcgtcgacccggtgaaggagccgatcccggtgcggccgaccgcgcattacacgatgggcggcatcgaaaccgaccagaactgcgaaacgcgcatcaagggcctgttcgcggtgggcgagtgcagctccgtcggcctccacggcgcgaaccgcctgggcagcaatagcctcgccgaactcgtggtcttcggccggctggccggcgagcaggccacggaacgggcggccaccgcgggcaacggcaacgaggccgccatcgaggcccaggccgccggcgtggaacagcggctgaaggatctggtcaaccaggatggcggcgaaaactgggcgaagatccgcgacgaaatgggcctggcgatggaagagggctgcggcatctaccgcacgccggagctgatgcagaagaccatcgataagctggcggaactgcaggaacggttcaaacgggtccggatcacggatacctcctcggtgttcaacaccgacctcctgtacaccatcgagctgggccacggcctcaacgtggcggagtgtatggcgcactccgcgatggcgcggaaggaaagccgcggtgcccaccagcgcctcgacgaaggctgcaccgagcgcgacgatgtgaacttcctcaaacacacgctggccttccgggacgcggatggcacgacccgcctcgaatatagcgatgtgaagatcacgaccctgcctcctgcgaagcgcgtctacggcggtgaagccgacgcggcagacaaggccgaggcagccaataagaaggagaaagccaatggctga

SEQ ID NO: 53 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 54 - DNA, 735 bp, Escherichia coli
  source 1..735: mol_type = genomic DNA
atggcggaaatgaagaacctgaagatcgaggtcgtccggtataatccggaggtggataccgccccccacagcgcgttctacgaagtcccgtacgacgccaccaccagcctcctggatgcgctgggctacatcaaggataacctggcccccgatctgtcgtaccgctggtcctgtcgcatggccatctgtggctcctgcggcatgatggtgaataacgtgccgaagctggcctgcaagaccttcctgcgggactataccgacggcatgaaagtggaggccctcgcgaacttcccgatcgagcgcgacctggtggtcgatatgacgcacttcatcgagagcctggaagcgatcaaaccctatatcatcggcaacagccggaccgccgaccagggcacgaacatccagacgcccgcgcagatggcgaagtatcaccagttctccggctgtatcaattgcggcctgtgctatgcggcgtgcccgcagttcggcctgaaccccgagttcatcggcccggcggccatcacgctcgcccaccgctacaacgaggacagccgcgatcacggcaagaaggagcgcatggcccagctcaattcgcagaatggcgtgtggagctgcaccttcgtgggctattgctccgaggtctgccccaagcatgtcgacccggcggcggcgatccagcagggcaaggtcgagtcgtcgaaagacttcctgatcgccacgctcaagccgcggtga

SEQ ID NO: 55 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 56 - DNA, 396 bp, Escherichia coli
  source 1..396: mol_type = genomic DNA
atgacgaccaagcgcaagccgtacgtccggccgatgacctcgacctggtggaaaaagctccccttctaccgcttctatatgctgcgcgagggcaccgcggtgccggccgtctggttctcgatcgaactgatcttcggcctgttcgcgctgaagaatggcccggaggcgtgggccggcttcgtggacttcctgcagaaccccgtcatcgtcatcatcaatctcatcacgctcgccgcggccctcctgcacaccaagacctggttcgagctggccccgaaggcggcgaacatcatcgtgaaggacgagaagatgggcccggagccgatcatcaaatccctgtgggcggtcacggtggtcgccacgatcgtcatcctgttcgtggcgctgtactggtga

SEQ ID NO: 57 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 58 - DNA, 360 bp, Escherichia coli
  source 1..360: mol_type = genomic DNA
atgatcaatccgaaccccaaacggagcgacgagcccgtcttctggggcctgttcggcgcgggcggcatgtggagcgccatcatcgcgccggtcatgatcctcctcgtgggcatcctcctccccctgggcctgttccccggcgacgccctgtcgtacgagcgcgtgctggccttcgcccagtccttcatcggccgcgtgttcctcttcctcatgatcgtgctccccctctggtgcggcctgcaccgcatgcaccacgccatgcacgacctgaagatccatgtccccgccggcaagtgggtcttctacggcctcgcggcgatcctgacggtcgtgacgctgatcggcgtggtcacgatctga